SEQUENCE LISTING (24 sequences)

SEQ ID NO: 1 - DNA, 21 bp, synthetic construct
  source 1..21: mol_type = other DNA
tcgacttcccctcttccgatg

SEQ ID NO: 2 - DNA, 22 bp, synthetic construct
  source 1..22: mol_type = other DNA
ctcaggttctgggagagggtag

SEQ ID NO: 3 - DNA, 21 bp, synthetic construct
  source 1..21: mol_type = other DNA
gagcctagggccgggattctc

SEQ ID NO: 4 - DNA, 846 bp, synthetic construct
  source 1..846: mol_type = other DNA
atgagtcagcagcggccggcgaggaagttacccagtctcctcctggacccgacggaggagacggttcgccgtcggtgccgagaccccatcaacgtggagggcctgctgccatcaaaaataaggattaatttagaagataatgtacaatatgtgtccatgagaaaagctctaaaagtgaagagacctcgttttgatgtatcgctggtttatttaactcgaaaatttatggatcttgtcagatctgctcccgggggtattcttgacttaaacaaggttgcaacgaaactgggagtccgaaagcggagagtgtatgacatcaccaatgtcttagatggaatcgacctcgttgaaaagaaatccaagaaccatattagatggataggatctgatcttagcaattttggagcagttccccaacaaaagaagctacaggaggaactttctgacttatcagcaatggaagatgctttggatgagttaattaaggattgtgctcagcagctgtttgagttaacagatgacaaagaaaatgaaagactagcatatgtgacctatcaagacattcatagcattcaggccttccatgaacagatcgtcattgcagttaaagctccagcagaaaccagattggatgttccagctcccagagaagactctatcacagtgcacataaggagcaccaacggacctatcgatgtctatttgtgtgaagtggagcagggtcagaccagtaacaaaaggtctgaaggtgtcgggacctcttcatctgagagcactcatccagaaggccctgaggaagaagaaaatcctcagcaaagtgaagaattgcttgaagtaagcaactga

SEQ ID NO: 5 - protein, 281 aa, Homo sapiens
MSQQRPARKLPSLLLDPTEETVRRRCRDPINVEGLLPSKIRINLEDNVQYVSMRKALKVKRPRFDVSLVYLTRKFMDLVRSAPGGILDLNKVATKLGVRKRRVYDITNVLDGIDLVEKKSKNHIRWIGSDLSNFGAVPQQKKLQEELSDLSAMEDALDELIKDCAQQLFELTDDKENERLAYVTYQDIHSIQAFHEQIVIAVKAPAETRLDVPAPREDSITVHIRSTNGPIDVYLCEVEQGQTSNKRSEGVGTSSSESTHPEGPEEEENPQQSEELLEVSN

SEQ ID NO: 6 - DNA, 789 bp, synthetic construct
  source 1..789: mol_type = other DNA
atgggaagtgaaagagctctcgtactggacagattagcaagcaatgtggcaaaacgaaaaagctcaatgcctcagaaattcattggtgagaagcgccactgctttgatgtcaactataattcaagttacatgtatgagaaagagagtgagctcatacagacccgcatgatggaccaagccatcaataacgccatcagctatcttggcgccgaagccctgcgccccttggtccagacaccgcctgctcccacctcggagatggttccagttatcagcagcatgtatcccatagccctcacccgggctgagatgtcaaacggtgcccctcaagagctggaaaagaaaagcatccaccttccagagaagagcgtgccttctgagagaggcctctctcccaacaatagtggccacgactccacggacactgacagcaaccatgaagaacgccagaatcacatctatcagcaaaatcacatggtcctgtctcgggcccgcaatgggatgccacttctgaaggaggttccccgctcttacgaactcctcaagcccccgcccatctgcccaagagactccgtcaaagtgatcaacaaggaaggggaggtgatggatgtgtatcggtgtgaccactgccgcgtcctcttcctggactatgtgatgttcacgattcacatgggctgccacggcttccgtgaccctttcgagtgtaacatgtgtggatatcgaagccatgatcggtatgagttctcgtctcacatagccagaggagaacacagagccctgctgaagtga

SEQ ID NO: 7 - protein, 262 aa, Homo sapiens
MGSERALVLDRLASNVAKRKSSMPQKFIGEKRHCFDVNYNSSYMYEKESELIQTRMMDQAINNAISYLGAEALRPLVQTPPAPTSEMVPVISSMYPIALTRAEMSNGAPQELEKKSIHLPEKSVPSERGLSPNNSGHDSTDTDSNHEERQNHIYQQNHMVLSRARNGMPLLKEVPRSYELLKPPPICPRDSVKVINKEGEVMDVYRCDHCRVLFLDYVMFTIHMGCHGFRDPFECNMCGYRSHDRYEFSSHIARGEHRALLK

SEQ ID NO: 8 - DNA, 291 bp, synthetic construct
  source 1..291: mol_type = other DNA
atgagcgataacgatgacatcgaggtggagagcgacgaagagcaaccgaggtttcaatctgcggctgacaaacgggctcatcataatgcactggaacgaaaacgtagggaccacatcaaagacagctttcacagtttgcgggactcagtcccatcactccaaggagagaagctctatttcctcttttggaaattgtgtactcctgtccttcatcgtcaaagtttgatgcagaaatgccacaccttcatttcaagctaccaagtgcacaagaaaaaagaatgcaagatttaa

SEQ ID NO: 9 - protein, 96 aa, Homo sapiens
MSDNDDIEVESDEEQPRFQSAADKRAHHNALERKRRDHIKDSFHSLRDSVPSLQGEKLYFLFWKLCTPVLHRQSLMQKCHTFISSYQVHKKKECKI

SEQ ID NO: 10 - DNA, 1836 bp, synthetic construct
  source 1..1836: mol_type = other DNA
atgctggagaactacaggaacctggtctcagtggaacatcagctttccaaaccagatgtggtatctcagttagaggaggcagaagatttctggccagtggagagaggaattcctcaagacaccattccagagtatcctgagctccagctggaccctaaattggatcctcttcctgctgagagtcccctaatgaacattgaggttgttgaggtcctcacactgaaccaggaggtggctggtccccggaatgcccagatccaggccctatatgctgaagatggaagcctgagtgcagatgcccccagtgagcaggtccaacagcagggcaagcatccaggtgaccctgaggccgcgcgccagaggttccggcagttccgttataaggacatgacaggtccccgggaggccctggaccagctccgagagctgtgtcaccagtggctacagcctaaggcacgctccaaggagcagatcctggagctgctggtgctggagcagttcctaggtgcactgcctgtgaagctccggacatgggtggaatcgcagcacccagagaactgccaagaggtggtggccctggtagagggtgtgacctggatgtctgaggaggaagtacttcctgcaggacaacctgccgagggcaccacctgctgcctcgaggtcactgcccagcaggaggagaagcaggaggatgcagccatctgcccagtgacagtgctccctgaggagccagtgaccttccaggatgtggctgtggacttcagccgggaggagtgggggctgctgggcccgacacagaggaccgagtaccgcgatgtgatgctggagacctttgggcacctggtctctgtggggtgggagactacactggaaaataaagagttagctccaaattctgacattcctgaggaagaaccagcccccagcctgaaagtacaagaatcctcaagggattgtgccttgtcctctacattagaagataccttgcagggtggggtccaggaagtccaagacacagtgttgaagcagatggagtctgctcaggaaaaagaccttcctcagaagaagcactttgacaaccgtgagtcccaggcaaacagtggtgctcttgacacaaaccaagtttcgctccagaaaattgacaaccctgagtcccaggcaaacagtggcgctcttgacacaaaccaagttttgctccacaaaattcctcctagaaaacgattgcgcaaacgtgactcacaagttaaaagtatgaaacataattcacgtgtaaaaattcatcagaagagctgtgaaaggcaaaaggccaaggaaggcaatggttgtaggaaaaccttcagtcggagtactaaacagattacgtttataagaattcacaaggggagccaagtttgccgatgcagtgaatgtggtaaaatattccggaacccaagatacttttctgtgcataagaaaatccataccggagagaggccctatgtgtgtcaagactgtgggaaaggatttgttcagagctcttccctcacacagcatcagagagttcattctggagagagaccatttgaatgtcaggagtgtgggaggaccttcaatgatcgctcagccatctcccagcacctgaggactcacactggcgctaagccctacaagtgtcaggactgtggaaaagccttccgccagagctcccacctcatcagacatcagaggactcacaccggggagcgcccatatgcatgcaacaaatgtggaaaggccttcacccagagctcacaccttattgggcaccagagaacccacaataggacaaagcgaaagaagaaacagcctacctcatag

SEQ ID NO: 11 - protein, 611 aa, Homo sapiens
MLENYRNLVSVEHQLSKPDVVSQLEEAEDFWPVERGIPQDTIPEYPELQLDPKLDPLPAESPLMNIEVVEVLTLNQEVAGPRNAQIQALYAEDGSLSADAPSEQVQQQGKHPGDPEAARQRFRQFRYKDMTGPREALDQLRELCHQWLQPKARSKEQILELLVLEQFLGALPVKLRTWVESQHPENCQEVVALVEGVTWMSEEEVLPAGQPAEGTTCCLEVTAQQEEKQEDAAICPVTVLPEEPVTFQDVAVDFSREEWGLLGPTQRTEYRDVMLETFGHLVSVGWETTLENKELAPNSDIPEEEPAPSLKVQESSRDCALSSTLEDTLQGGVQEVQDTVLKQMESAQEKDLPQKKHFDNRESQANSGALDTNQVSLQKIDNPESQANSGALDTNQVLLHKIPPRKRLRKRDSQVKSMKHNSRVKIHQKSCERQKAKEGNGCRKTFSRSTKQITFIRIHKGSQVCRCSECGKIFRNPRYFSVHKKIHTGERPYVCQDCGKGFVQSSSLTQHQRVHSGERPFECQECGRTFNDRSAISQHLRTHTGAKPYKCQDCGKAFRQSSHLIRHQRTHTGERPYACNKCGKAFTQSSHLIGHQRTHNRTKRKKKQPTS

SEQ ID NO: 12 - RNA, 22 nt, synthetic construct
  source 1..22: mol_type = other RNA
tccctgagaccctaacttgtga

SEQ ID NO: 13 - RNA, 23 nt, synthetic construct
  source 1..23: mol_type = other RNA
aaaagtgcttacagtgcaggtag

SEQ ID NO: 14 - RNA, 23 nt, synthetic construct
  source 1..23: mol_type = other RNA
caaagtgcttacagtgcaggtag

SEQ ID NO: 15 - RNA, 22 nt, synthetic construct
  source 1..22: mol_type = other RNA
cagtgcaatgttaaaagggcat

SEQ ID NO: 16 - RNA, 22 nt, synthetic construct
  source 1..22: mol_type = other RNA
cagtgcaatgatgaaagggcat

SEQ ID NO: 17 - RNA, 21 nt, synthetic construct
  source 1..21: mol_type = other RNA
tggtagactatggaacgtagg

SEQ ID NO: 18 - RNA, 22 nt, synthetic construct
  source 1..22: mol_type = other RNA
actgctgagctagcacttcccg

SEQ ID NO: 19 - RNA, 19 nt, synthetic construct
  source 1..19: mol_type = other RNA
atcccaccactgccaccat

SEQ ID NO: 20 - RNA, 23 nt, synthetic construct
  source 1..23: mol_type = other RNA
tgcaccatggttgtctgagcatg

SEQ ID NO: 21 - RNA, 22 nt, synthetic construct
  source 1..22: mol_type = other RNA
tgtaaacatcctacactcagct

SEQ ID NO: 22 - RNA, 22 nt, synthetic construct
  source 1..22: mol_type = other RNA
ataaagctagataaccgaaagt

SEQ ID NO: 23 - RNA, 23 nt, synthetic construct
  source 1..23: mol_type = other RNA
tctttggttatctagctgtatga

SEQ ID NO: 24 - RNA, 22 nt, synthetic construct
  source 1..22: mol_type = other RNA
agaggctggccgtgatgaattc

(19) United States
(12) Patent Application Publication
(10) Pub. No.: US 2023/0277600 A1
(43) Pub. Date: Sep. 7, 2023

(54) Treatment Of Age-Related White Matter Loss By Competitive Replacement Of Glial Cells

(71) Applicants: University of Rochester, Rochester, NY (US); University of Copenhagen, Copenhagen (DK)

(72) Inventors: Steven A. Goldman, Webster, NY (US); Ricardo da Costa Barbedo Vieira, Copenhagen (DK)

(73) Assignees: University of Rochester, Rochester, NY (US); University of Copenhagen, Copenhagen, NY (DK)

(21) Appl. No.: 18/047,758

(22) Filed: Oct. 19, 2022

Related U.S. Application Data
(60) Provisional application No. 63/257,767, filed on Oct. 20, 2021.

(51) Int. Cl. A61K 35/30; A61P 25/28

(52) U.S. Cl. CPC A61K 35/30; A61P 25/28

(57) ABSTRACT

The present application relates to alleviating adverse effects of oligodendrocyte loss, astrocyte loss, or white matter loss, including age-related oligodendrocyte loss, age-related astrocyte loss, or age-related white matter loss, in the brain of a subject. The present application also relates to rejuvenating a glial progenitor cell or a progeny thereof, or to enhancing the development potential of a glial progenitor cell or a progeny thereof.

### CROSS REFERENCE TO RELATED APPLICATION

This application claims priority to U.S. Provisional Application No. 63/257,767 filed on Oct. 20, 2021. The content of the application is incorporated herein by reference in its entirety.

### REFERENCE TO A SEQUENCE LISTING

This application incorporates by reference the Sequence Listing submitted in Computer Readable Form as file SeqListing16111803802, created on Oct. 18, 2022 and containing 26,806 bytes.

### FIELD

The present application relates to treatment of oligodendrocyte loss, astrocyte loss, or white matter loss, including age-related oligodendrocyte loss, age-related astrocyte loss, or age-related white matter loss.

### BACKGROUND

Age-related loss of white matter, oligodendrocyte, or astrocyte commonly occurs in older people and can lead to poor outcomes, including cognitive impairment, dementia, urinary incontinence, gait disturbances, depression, and increased risk of stroke and death. This loss involves partial loss of myelin, axons, and oligodendroglial cells; mild reactive astrocytic gliosis; sparsely distributed macrophages as well as stenosis resulting from hyaline fibrosis of arterioles and smaller vessels. Age-related white matter loss is generally regarded as a form of incomplete ischemia mainly related to cerebral small vessel arteriolosclerosis. Such small vessel alterations can result in damage to the blood-brain barrier and chronic leakage of fluid and macro-molecules in the white matter. Indeed, an increased concentration of cerebrospinal fluid albumin and IgG values were found in patients having age-related white matter loss. Although age-related white matter loss has been a significant problem clinically, there have been relatively few studies conducted to evaluate treatments for this condition.

The Epidemiology of Vascular Ageing MRI study has shown a positive linear relationship between blood pressure and the severity of age-related white matter loss severity. Statins have long been used to reduce cardiovascular events and ischemic stroke in coronary patients. However, it is uncertain whether statins are useful in treating age-related white matter loss. Acetylcholinesterase inhibitors (donepezil, galantamine, and rivastigmine) and N-methyl-D-aspartate (NMDA) receptor antagonists (memantine) have been approved for treatment of Alzheimer's Disease. There is also evidence that hyperhomocysteinemia is associated with age-related white matter loss. It is uncertain, however, whether homocysteine lowering therapy will be useful in slowing such white matter loss.

There is a need for therapeutics and methods for treating disorders and conditions mediated by or characterized by loss of white matter, oligodendrocytes, or astrocytes. The present disclosure is directed to overcoming these and other deficiencies in the art.

### SUMMARY

This disclosure addresses the need mentioned above in a number of aspects.

In some aspects, the disclosure provides a method of treating in a subject a condition mediated by age-related oligodendrocyte loss. The method comprises administering a therapeutically effective amount of a population of isolated glial progenitor cells to the subject in need thereof. The condition can be a vascular leukoencephalopathy, an adult-onset autoimmune demyelination condition, a chronic post-radiation induced demyelination condition, an adult-onset lysosomal storage disease, an adult-onset leukodystrophy, or cerebral palsy.

In another aspect, the disclosure provides a method of treating in a subject a condition mediated by age-related astrocyte loss. The method comprises administering a therapeutically effective amount of a population of isolated glial progenitor cells to the subject in need thereof. The condition can be amyotrophic lateral sclerosis, frontotemporal dementia, schizophrenia, Huntington disease, Alexander disease, or Vanishing White Matter Disease.

In yet another aspect, the disclosure provides a method of treating in a subject a condition mediated by age-related white matter loss. The method comprises administering a therapeutically effective amount of a population of isolated glial progenitor cells to the subject in need thereof. Examples of the condition can include a vascular leukoencephalopathy, an adult-onset autoimmune demyelination condition, a chronic post-radiation induced demyelination condition, an adult-onset lysosomal storage disease, an adult-onset leukodystrophy, cerebral palsy, amyotrophic lateral sclerosis, frontotemporal dementia, schizophrenia, Huntington disease, Alexander disease, and Vanishing White Matter Disease.

In each of the methods described above, the condition can be Huntington's disease or subcortical dementia. Examples of the vascular leukoencephalopathy include subcortical stroke, diabetic leukoencephalopathy, and hypertensive leukoencephalopathy. Examples of the adult-onset autoimmune demyelination condition include relapsing-remitting multiple sclerosis, chronic or progressive multiple sclerosis, neuromyelitis optica, transverse myelitis, and optic neuritis.

In some embodiments for each of the methods described above, the population of the isolated glial progenitor cells are younger than glial progenitor cells, oligodendrocytes, or astrocytes in the subject. In some embodiments, the population of the isolated glial progenitor cells or progenies thereof replace at least some of glial progenitor cells, oligodendrocytes, or astrocytes in the subject. In some embodiments, the population of the isolated glial progenitor cells or progenies thereof grow or proliferate or divide faster than glial progenitor cells, oligodendrocytes, or astrocytes in the subject. In some embodiments, the population of the isolated glial progenitor cells or progenies thereof have a higher level of MYC and YAP1 pathway activity than glial progenitor cells, oligodendrocytes, or astrocytes in the subject.

In some embodiments, the subject is a mammal such as a human. The population of the isolated glial progenitor cells can be derived from pluripotent stem cells. Examples of the pluripotent stem cells include embryonic stem cells and induced pluripotent stem cells. In some embodiments, the glial progenitor cells can be cells rejuvenated from glial cells (such as glial progenitor cells, astrocytes, or oligodendrocytes) as disclosed herein.

For each of the methods described above, the administering can be carried out by intraparenchymal, intracallosal, intraventricular, intrathecal, intracerebral, intracisternal, or intravenous transplantation. In some examples, the population of isolated glial progenitor cells or progenies can be administered to the forebrain, striatum, and/or cerebellum. The isolated glial progenitor cells or progenies can be heterologous, xenogenic, allogeneic, isogenic, or autologous to the subject.

In some other aspects, the disclosure provide a method of rejuvenating, or enhancing the development potential of, a glial progenitor cell or a progeny thereof. The method comprises suppressing in the glial progenitor cell or the progeny a transcription repressor selected from the group consisting of E2F6, ZNF274, MAX, and IKZF3. The glial progenitor cell can be an aged glial progenitor cell. The progeny can be an oligodendrocyte or an astrocyte. The suppressing step may comprise expressing or introducing in the glial progenitor cell or the progeny a suppressor of the transcription repressor.

In another aspect, the disclosure provides a cell prepared according to the method described above or progeny thereof. The disclosure also provides an isolated glial progenitor cell or a progeny thereof comprising a suppressor of a transcription repressor selected from the group consisting of E2F6, ZNF274, MAX, and IKZF3. In some embodiments, the isolated glial progenitor cell or progeny comprises an exogenous suppressor. That is the suppressor is exogenous to the cell or progeny.

In a further aspect, the disclosure provides a method of treating a condition mediated by white matter loss, oligodendrocyte loss, or astrocyte loss. The method comprises administering to a subject in need thereof (i) a therapeutically effective amount of a suppressor of a transcription repressor selected from the group consisting of E2F6, ZNF274, MAX, and IKZF3; and/or (ii) a therapeutically effective amount of the cell prepared according to the method described above or a progeny thereof; and/or (iii) a therapeutically effective amount of the suppressor-containing glial progenitor cell or progeny described above. In some embodiments, the white matter loss, oligodendrocyte loss, or astrocyte loss is age-related.

The subject can be a mammal such as a human.

In some embodiments, the suppressor comprises a small molecule compound, an oligonucleotide, a nucleic acid, a peptide, a polypeptide, a CRISPR/Cas system, or an antibody or an antigen-binding portion thereof. In some examples, the suppressor can be miRNA or siRNA molecule, or a CRISPR/Cas system, or antisense nucleic acid.

In some embodiments, the nucleic acid comprises or encodes a miRNA or siRNA molecule. In some examples, the miRNA or siRNA molecule comprises a sequence that is at least 70% (e.g., 70%, 75%, 80%, 85%, 90%, 95%, 96%, 97%, 98%, or 99%) identical to one selected from the group consisting of miR-125b-5p, miR-106a-5p, miR-17-5p, miR-130a-3p, miR-130b-3p, miR-379-5p, miR-93-3p, miR-1260b, miR-767-5p, miR-30b-5p, miR-9-3p, miR-9-5p, and miR-485-5p. Preferably, the miRNA or siRNA molecule comprises a sequence that is at least 70% (e.g., 70%, 75%, 80%, 85%, 90%, 95%, 96%, 97%, 98%, or 99%) identical to the sequence of one selected from the group consisting of miR-125b-5p, miR-106a-5p, miR-17-5p, miR-130a-3p, miR-130b-3p, miR-379-5p, and miR-485-5p.

In some embodiments, the suppressor comprises a CRISPR-Cas system.

In the methods described above, the suppressor can be administered by intraparenchymal, intracallosal, intraventricular, intrathecal, intracerebral, intracisternal, or intravenous administration to the subject having the condition. Examples of the condition include a lysosomal storage disease, an autoimmune demyelination condition (e.g., multiple sclerosis, neuromyelitis optica, transverse myelitis, and optic neuritis), a vascular leukoencephalopathy (e.g., subcortical stroke, diabetic leukoencephalopathy, hypertensive leukoencephalopathy, age-related white matter disease, and spinal cord injury), a radiation induced demyelination condition, a leukodystrophy (e.g., Pelizaeus-Merzbacher Disease, Tay-Sach Disease, Sandhoff's gangliosidoses, Krabbe's disease, metachromatic leukodystrophy, mucopolysaccharidoses, Niemann-Pick A disease, adrenoleukodystrophy, Canavan's disease, Vanishing White Matter Disease, and Alexander Disease), or periventricular leukomalacia or cerebral palsy. In some embodiments, the condition is Huntington's disease or subcortical dementia.

The administering can be carried out by intraparenchymal, intracallosal, intraventricular, intrathecal, intracerebral, intracisternal, or intravenous transplantation. In some embodiments, the cell or the isolated glial progenitor cell or progeny thereof can be administered to the forebrain, striatum, and/or cerebellum. The cell or the isolated glial progenitor cell or progeny thereof can be heterologous, xenogenic, allogeneic, isogenic, or autologous to the subject.

The details of one or more embodiments of the disclosure are set forth in the description below. Other features, objectives, and advantages of the disclosure will be apparent from the description and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows representative images of expression of WT-mCherry. CRISPR-mediated integration of transgenic reporter cassette into the AAVS1 safe harbor locus yields color-tagged WT that express mCherry. E1-3, exon 1-3; LHA, left homology arm; SA, splice acceptor site; T2A, 2A self-cleaving peptide; Puro, Puromycin resistance gene; pA, polyadenylation sequence; CAG, CAG promoter; RHA, right homology arm. Scale: 500 μm.

FIG. 1B shows representative images of expression of HD-EGFP. CRISPR-mediated integration of transgenic reporter cassette into the AAVS1 safe harbor locus yields color-tagged HD hESCs that express EGFP.

FIG. 1C shows the engineered WT and HD hESC lines' HTT CAG length and respective transgenic insert.

FIG. 1D shows a PCR screening strategy to assess transgene cassette integration and zygosity using primers dna 803, dna 804, and dna 1835, (SEQ ID NOs: 1-3). PCR screening shows that WT-EGFP, WT-mCherry and HD-EGFP integrated the transgenic cassette in the correct site, with WT-mCherry and WT-EGFP harboring a homozygous integration while HD-EGFP harbors a heterozygous integration. E1-3, exon 1-3; LHA, left homology arm; RHA, right homology arm.

FIG. 1E shows representative images of WT-mCherry and HD-EGFP expression in the brain. Immunostaining for OCT4 shows that pluripotency is maintained following transgene insert.

FIG. 2A shows representative karyotypes from WT-mCherry and HD-EGFP to assess acquired copy number variants (CNVs) and loss-of-heterozygosity regions (LOH). Karyotyping shows that no chromosomal abnormalities were acquired during the transgene integration process.

FIG. 2B shows example of aCGH profiling of a human chromosome 20 carrying an amplification commonly found in hESCs (within the dashed lines), known to impart a selective growth advantage to hESCs. No such mutation was detected in WT-EGFP, WT-mCherry or HD-EGFP hESCs.

FIG. 2C shows comparative aCGH profiles detected multiple mutations in the engineered lines, within and outside of normal range. None are expected to influence experimental outcomes.

FIG. 3A illustrates creation of HD-chimeric mice, differentiation process and phenotypic characterization prior to experimental grafting.

FIG. 3B shows phase-contrast images of WT-mCherry- and HD-EGFP glial cultures, both highly enriched in bipolar hGPCs at 150 DIV. Scale: 50 μm.

FIG. 3C shows flow cytometry of 150 DIV cell preparations (WT-mCherry, n=10; HD-EGFP, n=6) reveals high enrichment of CD140a (PDGFRα)⁺/CD44⁺ hGPCs, with the remainder comprised of less mature A2B5⁺ hGPCs and PDGFRα⁻/CD44⁺ astrocytes. Fluorescent reporter expression remained consistent throughout glial differentiation. Unpaired two-tailed t tests; data are shown as means±SEM.

FIG. 3D shows that immunocytochemistry confirmed the enrichment of PDGFRα⁺ hGPCs in cultures generated from both WT-mCherry and HD-EGFP hESCs. A fraction of these hGPCs differentiated into GFAP⁺ astrocytes. Scale: 100 μm.

FIGS. 3E, 3F, and 3G show percentages of cells expressing (A) the reporters, (B) PDGFRα⁺, and (C) GFAP in HD-chimeric mice, respectively.

FIG. 4A are representative images demonstrating human wildtype glia outcompeting and displacing previously integrated HD glia. Engraftment of WT glia (mCherry⁺, red) into the striatum of HD chimeras yielded progressive replacement of HD glia (EGFP⁺, green) creating extensive exclusive domains in their advance. Dashed outlines (white) demarcate the striatal outlines within which human cells were mapped and quantified. STR—striatum (caudate-putamen); LV—lateral ventricle; CTX—cortex. Dashed rectangle (orange) represents inset at 72 weeks. Left scale bars: 500 μm; Right scale bars 100 μm.

FIGS. 4B-4C are representative images demonstrating human wildtype glia outcompeting and displacing previously integrated HD glia. FIG. 4B demonstrates that these exclusive domains are formed as WT GPCs (Olig2+, white) displace their HD counterparts. Scale bar: 50 μm. FIG. 4C shows GPC replacement precedes astrocytic replacement, as within regions dominated by WT glia, HD astrocytes (hGFAP+, white) could be found. Scale bar: 10 μm.

FIGS. 4D-4E show human wildtype glia outcompeting and displacing previously integrated HD glia. FIG. 4D is a cartoon depicting the strategy employed to quantify distribution of human glia in the striatum over time. Human glia were mapped in 15 equidistant sections (5 are shown as example) of the murine striatum and reconstructed in 3D for analysis. Their distribution was measured radially as a function of distance to the injection site. FIG. 4E shows that WT glia increase their spatial dominance over time; WT vs. HD (HD vs WT Group)−54 n=8 for 54 weeks, n=7 for 72 weeks. Their advance was accompanied by a progressive eradication of HD glia relative to HD chimera controls; HD (HD vs WT Group).

FIG. 4F shows human wildtype glia outcompete and displace previously integrated HD glia. Volumetric quantification shows that WT glia increase their spatial dominance over time; WT vs. HD (HD vs WT Group)−54 n=8 for 54 weeks, n=7 for 72 weeks. Their advance was accompanied by a progressive eradication of HD glia relative to HD chimera controls; HD (HD vs WT Group)−n=8 for 54 weeks, n=7 for 72 weeks vs. HD Control—n=4 for both timepoints; Two-way ANOVA with Šidák's multiple comparisons test; Main effects are shown as numerical P values, while post-hoc comparisons are shown as: ****P<0.0001, ***P<0.001, **P<0.01, *P<0.05; Data is presented as means±s.e.m.

FIG. 5 illustrates the experimental design of the HD vs WT mouse and the HD control mouse.

FIGS. 6A-6C show human wildtype glia outcompete previously integrated human HD glia.

FIG. 6A provides stereological estimations demonstrate that the total number of HD glia progressively decreases relatively to HD chimera controls as WT glia expands within the humanized striatum; Two-way ANOVA with Šidák's multiple comparisons test.

FIGS. 6B and 6C show the proportion of GPCs (Olig2+, FIG. 6B) and astrocytes (GFAP+, FIG. 6C) in both populations was maintained as they competed for striatal dominance; HD Control−n=4 for both timepoints; WT Control−n=4 for 54 weeks, n=3 for 72 weeks; HD vs WT−n=5 for 54 weeks, n=3 for 72 weeks; Orange arrows point to co-labelled cells. Data shown as means±s.e.m with individual data points.

FIGS. 6D-6E shows representative images of HD glia (FIG. 6D) and WT glia (FIG. 6E) of WT glia expanded as Olig2+ (white) GPCs displacing their HD counterparts. Within areas where they became dominant, they further differentiated into hGFAP+ (white) astrocytes.

FIG. 7A illustrates the experimental design and analytic timepoints of the WT Control group.

FIG. 7B shows representative images of engraftment of WT glia (mCherry+, red) into the adult striatum of Rag1(−/−) mice yields substantial humanization of the murine striatum over time.

FIGS. 7C-7D are volumetric quantifications show that WT glia infiltrate and disperse throughout the murine striatum over time, and they do so more broadly than those grafted onto HD chimeras. WT (HD vs WT Group)—n=8 for 54 weeks, n=7 for 72 weeks vs WT Control—n=7 for 54 weeks, n=5 for 72 weeks; Two-way ANOVA with Šidák's multiple comparisons test; Main effects are shown as numerical P values; Data is presented as means±s.e.m. FIG. 7C shows WT control. FIG. 7D shows cells/mm³.

FIG. 8 illustrates the experimental design for mice that received a 1:1 mixture of mCherry-tagged (WT-mCherry) and untagged (WT-untagged) WT glia.

FIGS. 9A-9D show co-engrafted isogenic clones of wildtype glia thrive and admix while displacing HD glia.

FIG. 9A shows immunolabeling against human nuclear antigen (hN) shows that both WT-mCherry (mCherry+hN+, red, white) and WT-untagged (mCherry− EGFP− hN+, white) glia expanded within the previously humanized striatum, progressively displacing HD glia (EGFP+hN+, green, white). Scale bar 500 μm.

FIG. 9B shows vast homotypic domains were formed as mixed WT glia expanded and displaced resident HD glia. Scale bar 100 μm.

FIG. 9C shows isogenic WT-mCherry and WT-untagged were found admixing. Scale bar 100 μm.

FIG. 9D shows that within WT glia dominated domains, only more complex astrocyte-like HD glia could be found, typically within white matter tracts. Scale bar: 10 μm.

FIG. 10 shows quantification of the proportion of WT-mCherry and WT-untagged glia within the striatum showed no significant difference between the two populations at either quantified timepoint (n=6 for each timepoint); Two-way ANOVA with Šidák's multiple comparisons test; means±s.e.m.

FIG. 11 illustrates the experimental design for co-engrafting WT and HT glia in neonatal mice.

FIG. 12A, 12B, 12B′, and 12C show representative images of the proportion of WT and HD glia within the striatum in mice co-engrafted with WT and HT glia. The images show no significant growth advantage to either cell population; n=5; two-tailed paired t-test.

FIGS. 13A-13B demonstrates equal growth of neonatally engrafted WT and HD glia is sustained by equally proliferative Ki67⁺ (white) glial pools; HD Control—n=3; WT Control—n=4; HD vs WT— n=5; One-way ANOVA with Tukey's multiple comparisons test. FIG. 13A shows striatal occupancy. FIG. 13B shows relative amount of Ki67⁺ cells.

FIG. 14A shows the experimental design to demonstrate differences in cellular age are sufficient to drive human glial repopulation.

FIG. 14B shows differences in cellular age are sufficient to drive human glial repopulation.

FIGS. 15A-15D show murine chimeras with striata substantially humanized by HD glia were generated to provide an in vivo model by which to assess the replacement of diseased human glia by their healthy counterparts. hGPCs derived from mHtt-expressing hESCs engineered to express EGFP were implanted into the neostriatum of immunocompromised Rag1^{(-/-)} mice and monitored their expansion histologically.

FIG. 15A shows the experimental design and analytical endpoints.

FIG. 15B shows that neonatally engrafted HD glia (EGFP⁺, green) expand within the murine striatum yielding substantial humanization of the tissue over time. Dashed lines demarcate the striatal borders within which human cells were mapped and quantified. Scale: 500 μm. STR, neostriatum.

FIG. 15C shows that their expansion is concomitant with an increase in the number of HD glia harbored in the murine striatum over time. Data presented as means±s.e.m with individual data points (n=4). One-way ANOVA with Tukey's multiple comparisons test; 12 weeks (n=3), 24 weeks (n=3), 36 weeks (n=4).

FIG. 15D shows that their expansion is concomitant with an increase in the number of HD glia harbored in the murine striatum over time at the cost of their Ki67⁺ proliferative cell pool.

FIGS. 15E-15J show murine chimeras with striata substantially humanized by HD glia were generated to provide an in vivo model by which to assess the replacement of diseased human glia by their healthy counterparts. hGPCs derived from mHtt-expressing hESCs engineered to express EGFP were implanted into the neostriatum of immunocompromised Rag1^{(-/-)} mice and monitored their expansion histologically.

FIG. 15E shows strategy employed to assess the extent of striatal humanization 36 weeks following neonatal implantation of HD GPCs. HD cell distribution was mapped in 15 equidistant sagittal sections (5 are shown for example) and reconstructed in 3D for analysis.

FIG. 15F shows rendered example of a mapped and reconstructed striatum for volumetric analysis.

FIG. 15G shows volumetric quantification shows that by 36 weeks HD glia had expanded throughout whole striatum assuming a uniform distribution; Data are shown as mean (line) and individual data points (n=4). Data presented as means±s.e.m with individual data points (n=4).

FIG. 15H-J show that as they colonized the murine striatum, HD glia either expanded and persisted as Olig2⁺ GPCs (arrows point to Olig2⁺/EGFP⁺ (red/green) cells) or differentiated into hGFAP⁺ (red) astrocytes. Proliferating (Ki67⁺, red) HD glia can be found even after 36 weeks of expansion, albeit in decreased numbers (D). Scale: 10 μm. Data presented as means±s.e.m with individual data points (n=4)

FIGS. 16A, 16B, 16B′, and 16C show proliferative advantage drives WT glia to advance through the humanized HD striatum.

FIGS. 17A, 17B, 17C, 17D, 17E, 17F, 17G, 17H and 17I show differences in cellular age are sufficient to drive competitive glial repopulation.

FIG. 17A shows an experimental design and analytical endpoints.

FIG. 17B shows that engraftment of younger WT glia (EGFP⁺, green) into the striatum of WT chimeras yielded selective replacement of their aged counterparts (mCherry⁺, red). Dashed outlines demarcate the striatal regions within which human cells were mapped and quantified. STR, striatum (caudate-putamen); LV, lateral ventricle; CTX, cortex. Scale: 500 μm.

FIG. 17C shows WT chimeric control, engrafted only at birth. Scale: 100 μm.

FIG. 17D shows rendered examples of mapped striata. Volumetric quantification shows that the younger WT glia replace their older isogenic counterparts as they expand from their injection site.

FIG. 17E shows results of Aged vs. Young (Isograft), n=3. Their advance tracked the progressive elimination of aged WT glia from the tissue, relative to control WT chimeras (Aged control). Scale: 100 μm.

FIG. 17F shows results of Aged (Isograft) vs. Aged (Control) n=3 each; 2-way ANOVA with Šidák's multiple comparisons test; Interactions or main effects are shown as numerical P values, while post-hoc comparisons are shown as: ****P<0.0001, ***P<0.001, **P<0.01, *P<0.05; data presented as means±SEM.

FIG. 17G shows that at the interface between young and aged WT glia, a higher incidence of Ki67⁺ (white) cells can be seen within the younger population. Dashed square represents inset color split (FIG. 17H). Scale: 50 μm

FIG. 17I. shows quantification of Ki67⁺ cells, indicating that younger WT glia are significantly more proliferative than their aged counterparts; n=3 for all experimental groups; One-way ANOVA with Šidák's multiple comparisons test; data are shown as means±SEM with individual data points.

FIG. 18A shows gating strategy flow cytometry analysis of WT-mCherry hESC lines.

FIG. 18B shows gating strategy flow cytometry analysis of HD-EGFP hESC lines. From dissociated glial cultures, live cells were identified by their lack of DAPI incorporation. Of these, cells stained for PDGFRα, CD44, PDGFRα/CD44 and A2B5 were identified based on antibody-specific fluorescence intensity, relative to their respective unstained gating controls. Essentially all cells retained their respective reporter expression throughout glial differentiation in vitro.

FIG. 19A shows that at the boundary between WT and HD glia, a high incidence of Ki67⁺ (white) cells can be seen exclusively within the WT glial population. I′. Higher magnification of two WT daughter cells at the edge of the competitive boundary.

FIG. 19B shows quantification of Ki67⁺ glia within each population as a function of time shows a significant proliferative advantage by WT glia, that is sustained throughout the experiment. HD control: 54 wks (n=4), 72 wks (n=4); WT control: 54 wks (n=5), 72 wks: n=3; WT vs. HD allograft: 54 wks (n=5), 72 wks (n=3). Comparisons by 2-way ANOVA with Šidák's multiple comparisons tests; mean±SEM.

FIG. 20A-20I show WT glia acquire a dominant competitor transcriptional profile in the face of resident HD glia.

FIG. 20A shows an experimental design.

FIG. 20B shows uniform manifold approximation projection (UMAP) visualization of the integrated scRNA-seq data identifying six major cell populations.

FIG. 20C shows UMAP visualization of the split by group scRNA-seq data identifying the six major cell populations.

FIG. 20D shows stacked bar plot proportions of cell types in each group.

FIG. 20E shows cell cycle analysis notched box plots of cycling GPCs and GPCs in the G2/M phase. The box indicates the interquartile range, the notch indicates the 95% confidence interval with the median at the center of the notch, and the error bars represent the minimum and maximum non-outlier values. Comparisons between groups utilized Dunn tests following a Kruskal-Wallis test with multiple comparisons adjusted via the Benjamini-Hochberg method. *=<0.05, **<0.01, ***=<0.001, ****=<0.0001 adjusted p-value.

FIG. 20F shows Venn diagram of pairwise differentially expressed GPC genes (Log 2 fold change >0.15, adjusted p-value<0.05).

FIG. 20G shows curated ingenuity pathway analysis of genes differentially expressed between GPC groups. The size of circles represent p-value while the shading indicates activation Z-Score with red being more active in the upper group and green being more active in the lower group.

FIG. 20H shows a heatmap of curated pairwise differentially expressed GPC genes.

FIG. 20I shows violin plots of pairwise differentially expressed GPC ribosomal gene log 2 fold changes.

FIG. 21A-21I show that WT glia acquire a dominant transcriptional profile when confronting their aged counterparts.

FIG. 21A shows the experimental design.

FIG. 21B shows UMAP visualization of the integrated scRNA-seq data identifying six major cell populations.

FIG. 21C shows UMAP visualization of the split by group scRNA-seq data identifying the six major cell populations.

FIG. 21D shows stacked bar plot proportions of cell types in each group.

FIG. 21E shows cell cycle analysis notched box plots of cycling GPCs and GPCs in the G2/M phase. The box indicates the interquartile range, the notch indicates the 95% confidence interval with the median at the center of the notch, and the error bars represent the minimum and maximum non-outlier values. Comparisons between groups utilized Dunn tests, following a Kruskal-Wallis test with multiple comparisons adjusted via the Benjamini-Hochberg method. *=<0.05, **<0.01, ***=<0.001, ****=<0.0001 adjusted p-value.

FIG. 21F shows Venn diagram of pairwise differentially expressed GPC genes (Log 2 fold change >0.15, adjusted p-value<0.05).

FIG. 21G shows curated Ingenuity Pathway analysis of genes differentially expressed between GPC groups. The size of circles represent p-value while the shading indicates activation Z-Score with red being more active in the upper group and green being more active in the lower group.

FIG. 21H shows a heatmap of curated pairwise differentially expressed GPC genes.

FIG. 21I shows violin plots of pairwise differentially expressed GPC ribosomal gene log 2 fold changes.

FIGS. 22A-22F show transcriptional signature of competitive advantage.

FIG. 22A shows schematic of transcription factor candidate identification.

FIG. 22B shows violin plots of identified WGCNA module eigengenes per condition. Represented are significant modules (black, green, blue, brown, red, cyan), whose members are enriched for the downstream targets of the five transcription factors in (FIG. 22E).

FIG. 22C shows relative importance analysis to estimate the differential contribution of each biological factor (age vs genotype) to each module eigengene.

FIG. 22D shows that gene set enrichment analysis (GSEA) highlighted those prioritized transcription factors whose regulons were enriched for upregulated genes in dominant young WT cells.

FIG. 22E shows important transcription factors predicted via SCENIC to establish competitive advantage and their relative activities across groups.

FIG. 22F shows regulatory network with represented downstream targets and their functional signaling pathways. Targets belong to highlighted modules in FIG. 22B, and their expressions are controlled by at least one other important transcription factors in FIG. 22E. NES: Network enrichment score.

FIGS. 23A, 23B, and 23C show that aged human glia are eliminated by their younger counterparts through induced apoptosis.

FIG. 23A shows that at the border between young (EGFP⁺, green) and aged WT glia (mCherry⁺, red), a higher incidence of apoptotic TUNEL⁺ (white) cells are apparent in the aged population. Scale: 100 μm.

FIG. 23B illustrates that higher magnification of a competitive interface between these distinct populations shows resident glia selectively undergoing apoptosis. Scale: 50 μm.

FIG. 23C illustrates that quantification of TUNEL⁺ cells shows significantly higher incidence of TUNEL⁺ cells among aged resident WT glia, relative to both their younger isogenic counterparts, and to aged WT chimeric controls not challenged with younger cells. Quantification was performed on pooled samples from 60 and 80 weeks timepoints (n=5 for all experimental groups). One-way ANOVA with Šidák's multiple comparisons test; data are shown as means±SEM with individual data points.

FIGS. 24A and 24B show isolation of implanted human cells from their chimeric hosts.

FIG. 24A is a schematic illustrating the experimental workflow involved in the isolation of human cells from the striata of their chimeric hosts.

FIG. 24B shows example of the gating strategy employed in the FACS enrichment of human cells extracted from dissociated chimeric striata. Live cells were identified by their lack of DAPI incorporation. Of these, human cells were sorted based on their expression of their respective fluorescent reporter (EGFP⁺ or mCherry⁺), and harvested for single-cell sequencing and downstream analysis.

FIGS. 25A, 25B, 25C, 25D, 25E, and 25F show bulk RNA-Seq characterization of human fetal GPCs.

FIG. 25A shows a workflow of bulk and scRNA-Sequencing of CD140a+, CD140a−, and A2B5+/PSA-NCAM—selected 2nd trimester human fetal brain isolates.

FIG. 25B shows principal component analysis of all samples across two batches.

FIG. 25C shows a Venn diagram of CD140a+ vs CD140a− and CD140+ vs A2B5+/PSA-NCAM− differentially-expressed gene sets (p<0.01 and absolute log 2-fold change >1).

FIG. 25D shows Significant Ingenuity Pathway Analysis terms for both gene sets. Size represents −log 10 p-value and color represents activation Z-Score (Blue, CD140a+; Red, A2B5+ or CD140a−).

FIG. 25E shows log 2-fold changes of significant genes for both genesets. Missing bars were not significant.

FIG. 25F shows a heatmap of transformed transcripts per million (TPM) of selected genes in 1E.

FIGS. 26A, 26B, 26C, 26D, 26E, 26F, 26G and 26H show single cell RNA-sequencing of CD140a and A2B5 selected human fetal GPCs

FIG. 26A shows a UMAP plot of the primary cell types identified during scRNA-Seq analysis of FACS isolated hGPCs derived from 20 week gestational age human fetal VZ/SVZ.

FIG. 26B shows a UMAP of only PSA-NCAM⁻/A2B5⁺ human fetal cells.

FIG. 26C shows a UMAP of only CD140a⁺ human fetal cells.

FIG. 26D shows violin plots of cell type-selective marker genes.

FIG. 26E shows a volcano plot of GPC vs pre-GPC populations.

FIG. 26F shows feature plots of select differentially expressed genes between GPCs and pre-GPCs.

FIG. 26F shows select significantly-enriched GPC and pre-GPC IPA terms, indicating their −log 10 p-value and activation Z-Score.

FIG. 26H shows select feature plots of transcription factors predicted to be significantly activated in fetal hGPCs. Relative transcription factor regulon activation is displayed as calculated using the SCENIC package.

FIGS. 27A, 27B, 27C, 27D, 27E, and 27F show that adult human GPCs are transcriptionally and functionally distinct from fetal GPCs

FIG. 27A shows a workflow of bulk RNA-Seq analysis of human adult and fetal GPCs.

FIG. 27B shows principal component analysis of all samples across three batches.

FIG. 27C shows a Venn Diagram of both Adult vs Fetal differential expression gene sets.

FIG. 27B shows an IPA network of curated terms and genes. Node size is proportionate to node degree. Label color corresponds to enrichment in either adult (red) or fetal (blue) populations.

FIG. 27E shows bar plots of significant IPA terms by module. Z-Scores indicate predicted activation in fetal (blue) or adult (red) hGPCs.

FIG. 27F shows a bar plot of log 2-fold changes and heatmap of network genes' TPM.

FIGS. 28A, 28B, 28C, 28D, 28E, 28F, and 28G show that inference of transcription factor activity implicates a set of transcriptional repressors in the establishment of adult hGPC identity.

FIG. 28A shows that normalized enrichment score plots of significantly enriched transcription factors predicted to be active in fetal and adult GPCs. Each dot is a motif whose size indicates how many genes in which that motif is predicted to be active, and the color represents the window around the promoter at which that motif was found enriched.

FIG. 28B shows a heatmap of enriched TF TPMs

FIG. 28C shows log-fold changes vs adult GPCs, for both fetal hGPC isolates.

FIGS. 28D-G show predicted direct transcription factor activity of curated genes split into: (FIG. 28 D) fetal activators; (FIG. 28E) fetal repressors; (FIG. 28F) adult activators; and (FIG. 28G) adult repressors. Color indicates differential expression in either adult (red) or fetal (blue) hGPCs; shape dictates type of node (octagon, repressor; rectangle, activator; oval, other target gene). Boxed and circled genes indicate functionally-related genes contributing to either glial progenitor/oligodendrocyte identity, senescence/proliferation targets, or upstream or downstream TFs that were also deemed activated.

FIGS. 29A, 29B, 29C, and 29D show induction of an aged GPC transcriptome via adult hGPC-enriched repressors.

FIG. 29A shows a schematic outlining the structure of four distinct doxycycline (Dox)-inducible EGFP lentiviral expression vectors, each encoding one of the transcriptional repressors: E2F6, IKZF3, MAX, or ZNF274.

FIG. 29B shows that induced pluripotent stem cell (iPSC)-derived hGPC cultures (line C27) were transduced with a single lentivirus or vehicle for one day, and then treated with Dox for the remainder of the experiment. At 3, 7, and 10 days following initiation of Dox-induced transgene expression, hGPCs were isolated via FACS for qPCR.

FIG. 29C illustrates qPCRs of Dox-treated cells showing expression of each transcription factor, vs matched timepoint controls.

FIG. 29D shows qPCR fold-change heatmap of select aging related genes. Within timepoint comparisons to controls were calculated via post hoc least-squares means tests of linear models following regression of a cell batch effect. FDR adjusted p-values: *<0.05, **<0.01, ***<0.001.

FIGS. 30A, 30B, 30C, 30D, and 30E show that miRNAs drive adult GPC transcriptional divergence in parallel to transcription factor activity.

FIG. 30A shows principal component analysis of miRNA microarray samples from human A2B5+ adult and CD140a+ fetal GPCs.

FIG. 30B shows log 2 fold change bar plots and heatmap of differentially expressed miRNAs.

FIG. 30C shows characterization bubble plot of enrichment of miRNAs, versus the average log 2 FC of its predicted gene targets.

FIG. 30D shows curated signaling networks of fetal enriched miRNAs and their predicted targets.

FIG. 30E shows curated signaling networks of adult enriched miRNAs and their predicted targets.

FIGS. 31A, 31B, 31C, 31D, and 31E show enrichment of human fetal GPCs via CD140a+ or A2B5+/PSA-NCAM− selection.

FIG. 31A shows principal component analysis of CD140a+ and A2B5+ fetal GPCs.

FIG. 31B shows volcano plots indicating significant A2B5 (Green) and CD140a (Blue) enriched genes.

FIG. 31C shows principal component analysis of CD140a+ and CD140a− fetal cells.

FIG. 31D shows volcano plots indicating significant CD140a− (Magenta) and CD140a (Blue) enriched genes.

FIG. 31E shows upset plot of significant up and downregulated genes in both genesets.

FIGS. 32A, 32B, 32C, and 32D show single cell RNA-Seq quality filtering.

FIG. 32A shows violin plots of unfiltered A2B5⁺/PSA-NCAM⁻ captures.

FIG. 32B shows violin plots of unfiltered CD140a scRNA-seq captures.

FIG. 32C shows violin plots following quality filtration (Percent mitochondrial gene expression <15% and >500 unique genes) of A2B5⁺/PSA-NCAM⁻ captures.

FIG. 32D shows violin plots following quality filtration (Percent mitochondrial gene expression <15% and >500 unique genes) CD140a⁺ captures.

FIGS. 33A, 33B, and 33C show single cell RNA-sequencing of A2B5⁺/PSA-NCAM⁻ vs. CD140a⁻ fetal hGPCs. FIG. 33A shows UMAP plot of A2B5⁺ and CD140a⁻ fetal hGPCs.

FIG. 33B shows frequency of cell types in each sorting paradigm isolate. FIG. 33C shows scatter plot of differentially expressed bulk RNA-Seq log 2 fold changes vs pseudobulk log 2 fold changes between CD140a⁺ and A2B5⁺ fetal hGPC isolates.

FIG. 34 shows shared motifs of active transcription factors in fetal or adult hGPCs. Matrix of all predicted active transcription factors in fetal and adult GPCs. Size and color indicate degree of motifs that are shared between transcription factors.

FIG. 35 shows adult repressor isoform expression. Bar plots of transcripts per million (TPMs) of all protein coding adult repressor isoforms in each GPC group.

FIG. 36 shows bulk RNA-Seq of iPSC-derived hGPCs reveals concordant abundance of age-associated genes. iPSC-derived hGPCs (C27) were isolated via CD140a+ FACS and assayed via bulk RNA sequencing. Abundance of relevant glial age-associated genes, including those in an active transcription factor cohort, are displayed alongside fetal and adult hGPC data.

FIGS. 37A and 37B show transcription factor regulation of miRNAs provides post-transcriptional modulation of glial aging gene expression. FIG. 37A shows log 2 FC violin plots of significant adult vs fetal GPC transcription factors predicted to be upstream of differentially expressed adult vs fetal GPC miRNAs. FIG. 37B shows network of identified transcription factors from FIG. 26 and their predicted regulation of differentially expressed adult vs fetal hGPC miRNAs.

### DETAILED DESCRIPTION

This disclosure relates to compositions and methods for treating a condition mediated by oligodendrocyte loss, astrocyte loss, or white matter loss, including age-related oligodendrocyte loss, age-related astrocyte loss, or age-related white matter loss. This disclosure also relates to (a) rejuvenating a glial progenitor cell or a progeny thereof or (b) enhancing the development potential of a glial progenitor cell or a progeny thereof.

Conditions Mediated by Loss of while Matter/Oligodendrocytes/Astrocytes and Related Disorders

Certain aspects of this disclosure relate to compositions and methods for treating a condition or disorder mediated by oligodendrocyte loss, astrocyte loss, or white matter loss. Such a condition often entails a deficiency in myelin in central nerve system (“CNS”). Examples of such conditions or disorders include any diseases or conditions related to demyelination, insufficient myelination and remyelination, or dysmyelination in a subject. Such a condition or disorder can be inherited, acquired, or due to the ageing process, i.e., age-related. In some embodiments, the condition is that of age-related white matter disease defined as or characterized by oligodendrocyte loss, astrocyte loss, or white matter atrophy in the setting of normal otherwise healthy aging.

In humans, ageing represents the accumulation of changes in a human being over time and can encompass physical, psychological, and social changes. Ageing increases the risk of human diseases such as cancer, diabetes, cardiovascular disease, stroke, and many more, including demyelination in the CNS, which are often seen in various neurodegenerative diseases. Accordingly, in some embodiments of this disclosure, the condition or disorder is mediated by age-related oligodendrocyte loss, age-related astrocyte loss, or age-related white matter loss.

Demyelination in the CNS may occur in response to genetic mutation (leukodystrophies), autoimmune disease (e.g., multiple sclerosis), or trauma (e.g., traumatic brain injury, spinal cord injury, or ischemic stroke). Perturbation of myelin function may play a critical role in neurologic and psychiatric disorders such as Autism Spectrum Disorder (ASD), Alzheimer's disease, Huntington's disease, Multiple System Atrophy, Parkinson's disease, Fragile X syndrome, schizophrenia, and various leukodystrophies.

Leukodystrophies are a group of rare, primarily inherited neurological disorders that result from the abnormal production, processing, or development of myelin and are the result of genetic defects (mutations). Some forms are present at birth, while others may not produce symptoms until a child becomes older. A few primarily affect adults. Leukodystrophies include Canavan disease, Pelizaeus-Merzbacher disease, Hypomyelination with Atrophy of the Basal Ganglia and Cerebellum, Krabbe disease (Globoid cell leukodystrophy), X-linked adrenoleukodystrophy, Metachromatic leukodystrophy, Pelizaeus-Merzbacher-like disease (or hypomyelinating leukodystrophy-2), Niemann-Pick disease type C (NPC), Autosomal dominant leukodystrophy with autonomic diseases (ADLD), 4H Leukodystrophy (Pol III-related leukodystrophy), Zellweger Spectrum Disorders (ZSD), Childhood ataxia with central nervous system hypomyelination or CACH (also called vanishing white matter disease or VWMD), Cerebrotendinous xanthomatosis (CTX), Alexander disease (AXD), SOX10-associated peripheral demyelinating neuropathy, central dysmyelinating leukodystrophy, Waardenburg syndrome, Hirschsprung disease (PCWH), Adult polyglucosan body disease (APBD), Hereditary diffuse leukoencephalopathy with spheroids (HDLS), Aicardi-Goutieres syndrome (AGS), and Adult Refsum disease.

Suitable subjects for treatment in accordance with the methods described herein include any human subject having a condition mediated by a deficiency in myelin, which may be manifested by age-related oligodendrocyte loss, age-related astrocyte loss, or age-related white matter loss.

In another embodiment, the condition mediated by a deficiency in myelin is selected from the group consisting of pediatric leukodystrophies, the lysosomal storage diseases, congenital dysmyelination, cerebral palsy, inflammatory demyelination, post-infectious and post-vaccinial leukoencephalitis, radiation- or chemotherapy induced demyelination, and vascular demyelination.

In a further embodiment, the condition mediated by a deficiency in myelin requires myelination. In another embodiment, the condition mediated by a deficiency in myelin requires remyelination. In some embodiments, the condition requiring remyelination is selected from the group consisting of multiple sclerosis, neuromyelitis optica, transverse myelitis, optic neuritis, subcortical stroke, diabetic leukoencephalopathy, hypertensive leukoencephalopathy, age-related white matter disease, white matter dementia, Binswanger's disease, spinal cord injury, radiation- or chemotherapy induced demyelination, post-infectious and post-vaccinial leukoencephalitis, periventricular leukomalacia, and cerebral palsy.

In a further embodiment, the condition mediated by a deficiency in myelin is neurodegenerative disease. In some embodiments, the neurodegenerative disease is Huntington's disease. Huntington's disease is an autosomal dominant neurodegenerative disease characterized by a relentlessly progressive movement disorder with devastating psychiatric and cognitive deterioration. Huntington's disease is associated with a consistent and severe atrophy of the neostriatum which is related to a marked loss of the GABAergic medium-sized spiny projection neurons, the major output neurons of the striatum. Huntington's disease is characterized by abnormally long CAG repeat expansions in the first exon of the Huntingtin gene. The encoded polyglutamine expansions of mutant huntingtin protein disrupt its normal functions and protein-protein interactions, ultimately yielding widespread neuropathology, most rapidly evident in the neostriatum.

Other neurodegenerative diseases treatable in accordance with the present application include frontotemporal dementia, Alzheimer's disease, Parkinson's disease, multisystem atrophy, and amyotrophic lateral sclerosis.

In an embodiment, the condition mediated by a deficiency in myelin is a neuropsychiatric disease. In some embodiments, the neuropsychiatric disease is schizophrenia. Schizophrenia is a serious mental illness that affects how a person thinks, feels, and behaves. The symptoms of schizophrenia generally fall into the following three categories: (1) psychotic symptoms including altered perceptions, (2) negative symptoms including loss of motivation, disinterest and lack of enjoyment, and (3) cognitive symptoms including problems in attention, concentration, and memory. Other neuropsychiatric diseases treatable in accordance with the present application include autism spectrum disorder and bipolar disorder.

The above-described myelin-related disorders, inherited or acquired or age-related, impact millions of people, levying a heavy burden on affected individuals and their families. The pathological processes underlying many of these disorders remain poorly understood and few disease-modifying therapies exist. There are unmet needs for therapeutics for treating these disorders. This disclosure address these needs in a number of ways, such as competitive replacement of aged or older glial progenitor cells in the brain and rejuvenation of glial progenitor cells or their progeny cells.

Competitive Replacement of Glial Progenitor Cells in Adult Brain

Some aspects of this disclosure relate to competitive replacement of glial progenitor cells. Competition among cell populations in development and oncogenesis is well-established, and yet competition among cells in the adult brain has remained little-studied. In particular, it is unknown whether allografted human glia can outcompete diseased cells to achieve therapeutic replacement in the adult human brain.

As disclosed herein, inventors engrafted healthy, fluorophore-tagged wild-type (WT) hGPCs produced from human embryonic stem cells (hESCs), into the striata of adult mice that had been neonatally chimerized with spectrally-distinct mutant HTT-expressing hGPCs produced from Huntington disease (HD)-derived hESCs. The WT hGPCs outcompeted and ultimately eliminated their human HD counterparts, repopulating the host striata with healthy glia. Single-cell RNA-Seq revealed that WT donor hGPCs acquired a YAP1/MYC-defined dominant competitor phenotype upon interaction with the resident HD-derived glia. Competitive success depended primarily upon the age difference between competing populations, in that adult-transplanted WT hGPCs outcompeted resident isogenic WT cells that had been transplanted neonatally, and were thus older. These data indicate that aged and diseased human glia may be broadly replaced in adult brain by younger healthy hGPCs, and suggest that the transplantation of newly-generated glial progenitors may be used as a broad therapeutic platform for the replacement of aged as well as diseased human glia.

Glial dysfunction is a causal contributor to a broad spectrum of neurological conditions. Astrocytic and oligodendrocytic pathology have been associated with the genesis and progression of a number of both neurodegenerative and neuropsychiatric disorders, including conditions as varied as amyotrophic lateral sclerosis (ALS) and Huntington's disease (HD), as well as schizophrenia and bipolar disease. In such conditions, the replacement of diseased glia by healthy glial progenitor cells (hGPCs) might provide real therapeutic benefit, given their ability to disperse and colonize their hosts while giving rise to new astrocytes and oligodendrocytes. Yet, while human GPCs can outcompete and replace their murine counterparts in a variety of experimental therapeutic models, it has remained unclear if allografted human GPCs can replace other human cells, diseased or otherwise.

As disclosed in the examples below, human glial-chimeric mice were used to model competition between healthy and diseased human glia in vivo, by engrafting healthy hGPCs into the striata of adult mice neonatally chimerized with hGPCs derived from subjects with HD. HD is a prototypic monogenic neurodegenerative disease, resulting from the expression of a mutant, CAG-repeat expanded, Huntingtin (mHTT) gene.

Glial pathology is causally involved in the synaptic dysfunction of HD. Replacement of mHTT-expressing murine glia by implanted healthy hGPCs was sufficient to rescue aspects of HD phenotype in transgenic mouse models. As disclosed herein, inventors used genetically-tagged wild-type (WT) and mHTT-expressing hGPCs, derived from sibling lines of human embryonic stem cells (hESCs), to ask if healthy WT hGPCs can replace diseased HD hGPCs in vivo. It was found that when healthy hGPCs were delivered into the striata of adult mice chimerized with HD hGPCs, the healthy hGPCs outcompeted and displaced the already resident HD hGPCs. However, since the WT donor cells were effectively younger than the resident host glia that they were replacing, it was asked if differences in cell age might also contribute to competitive outcome. It was found this to be so, in that healthy young hGPCs implanted into adult mice that had been neonatally engrafted with separately-tagged glia derived from the same healthy line, inexorably replaced their older isogenic counterparts. Single cell RNA sequence analysis (scRNA-seq) of the younger winning and older losing hGPC populations revealed a set of differentially-expressed pathways that overlapped those of winning WT and losing HD hGPCs, suggesting a common transcriptional signature of competitively dominant GPCS. These data indicate that dynamic competition among clonally-distinct glial populations may occur in the mature adult brain, and that the replacement of both existing and diseased glia may thereby be achieved by the introduction of young healthy hGPCs.

In light of the contribution of glial pathology to a broad variety of neurodegenerative and neuropsychiatric disorders, inventors sought here to establish the relative fitness of wild-type and diseased human GPCs in vivo, so as to assess the potential for allogeneic glial replacement as a therapeutic strategy. Some parts of this disclosure focused on Huntington's disease, given the well-described role of glial pathology in HD. It was found that when WT hGPCs were introduced into brains already chimerized with HD hGPCs, the WT cells competitively dominated and ultimately replaced the already-resident HD glial progenitors. The selective expansion of the healthy cells was associated with the active elimination of the resident HD glia from the tissue, supported by the sustained proliferative advantage of the healthy donor cells relative to their already-resident diseased counterparts.

Single-cell RNA sequence analysis revealed that the dominance of healthy WT hGPCs encountering HD glia in vivo was associated with their expression of a signature typical of successful cell-cell competition. Surprisingly though, when controlled for the relative ages of the already-resident (older) and newly-introduced (younger) donor hGPCs, it was found that WT hGPCs transplanted into adult neostriata that had been chimerized neonatally, with separately-tagged but otherwise isogenic WT hGPCs, similarly dominated and replaced the already-resident hGPCs. This observation suggested that cellular youth was a critical determinant of competitive success, and of the ability of a donor hGPC population to replace that of the host. Accordingly, transplanted young WT hGPCs acquired the gene expression signature of a dominant competitor phenotype in vivo, whether challenged by already-resident older HD or isogenic WT hGPCs; indeed, the analysis described herein suggested that cellular youth was an even stronger determinant of competitive fitness than was disease genotype.

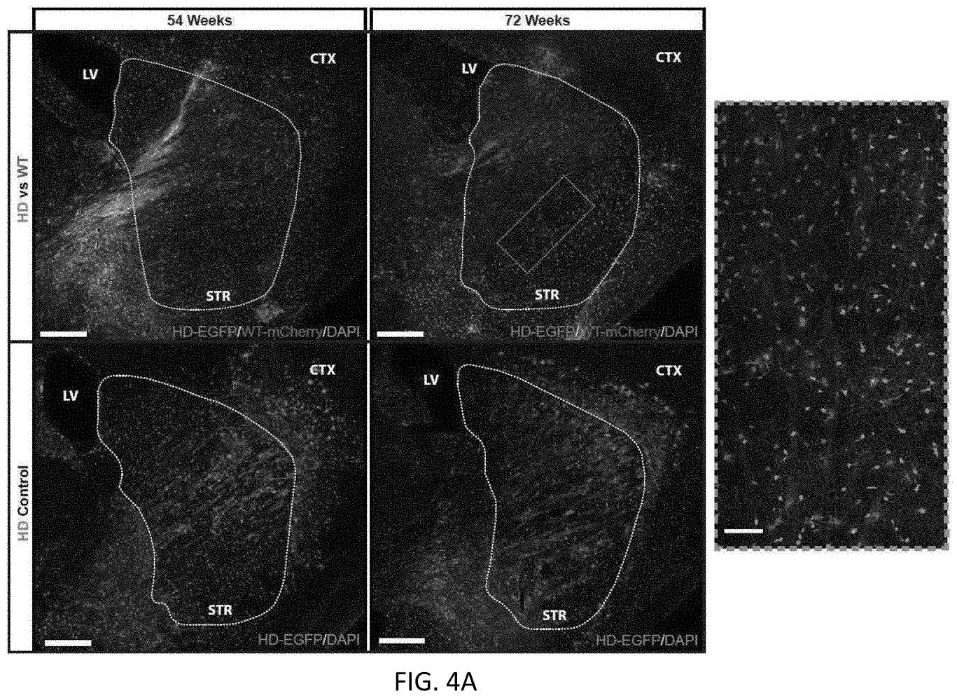

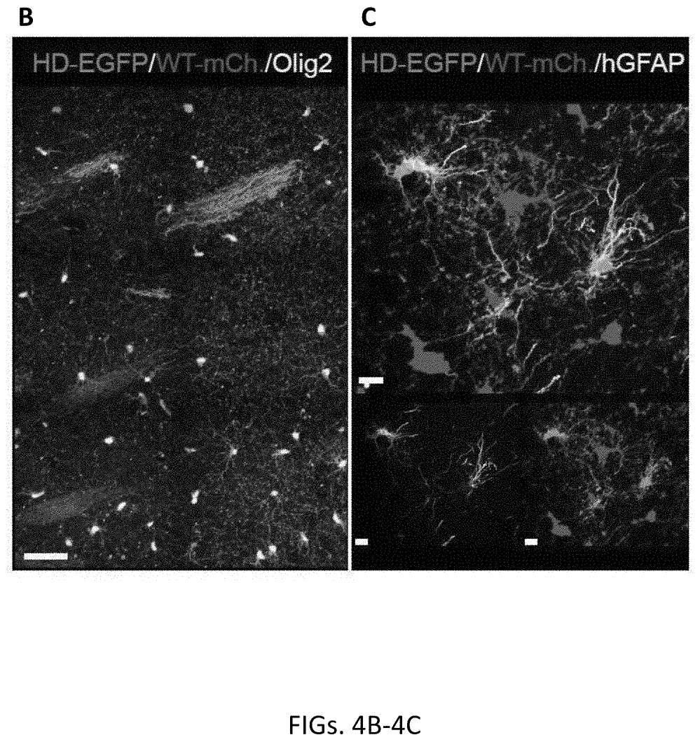

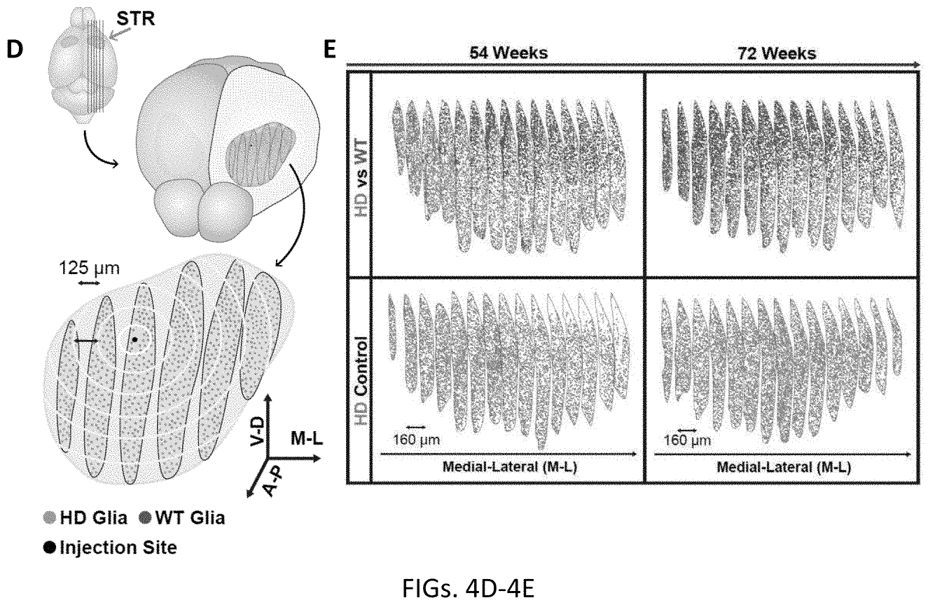

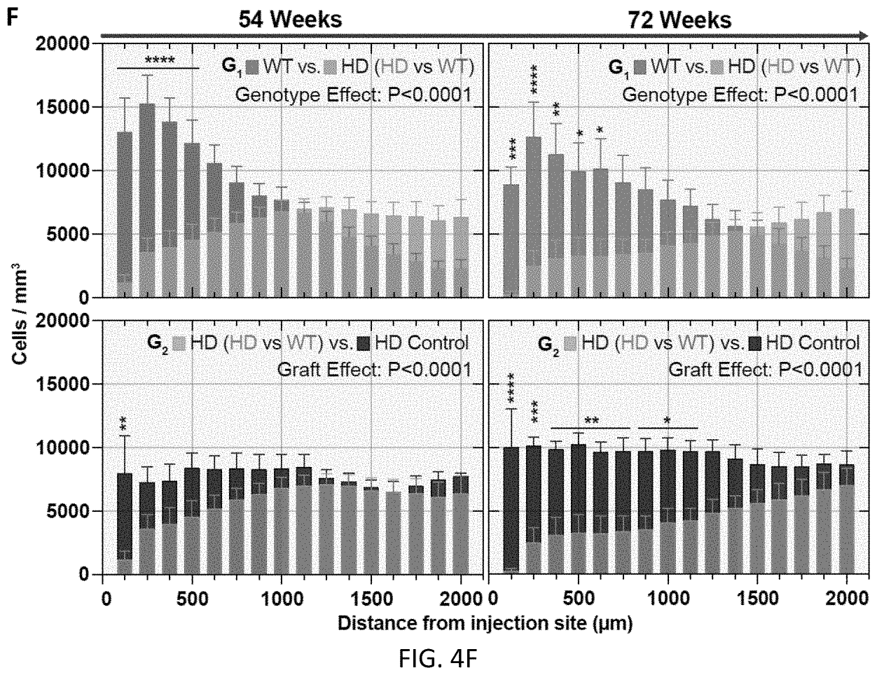

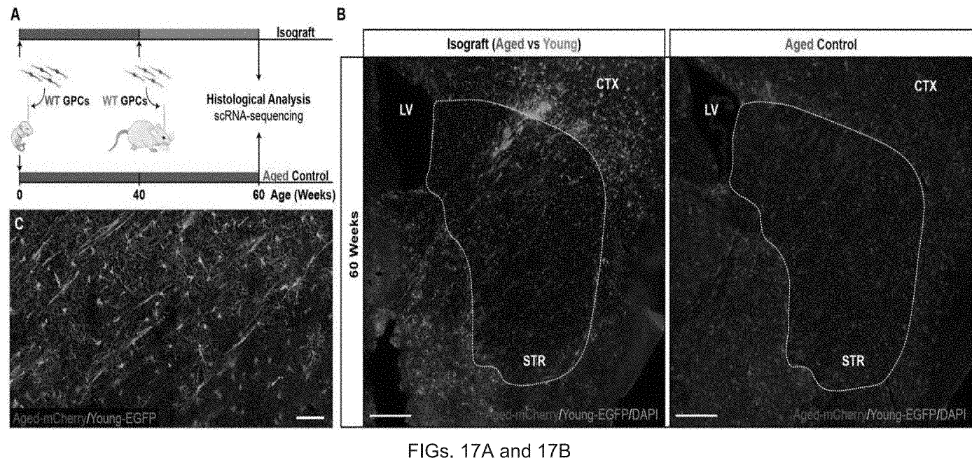

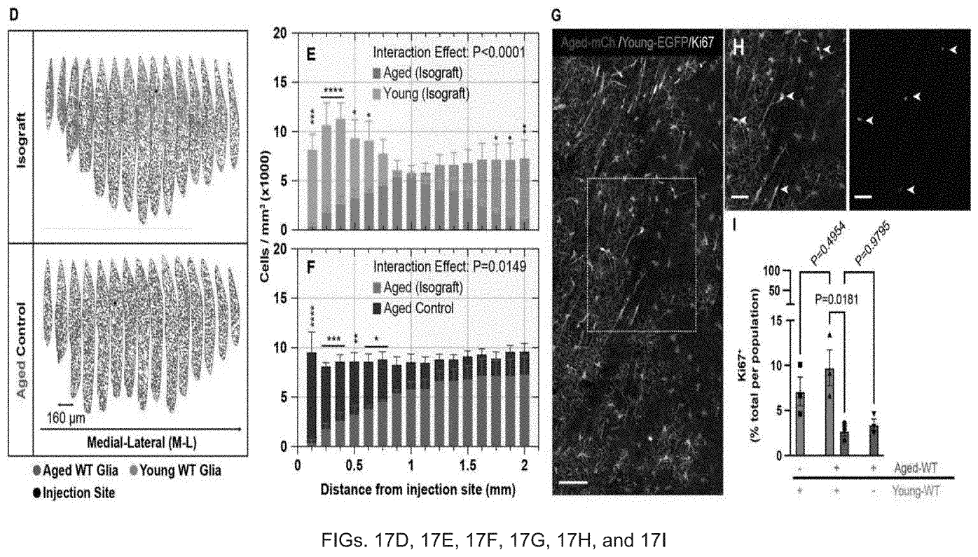

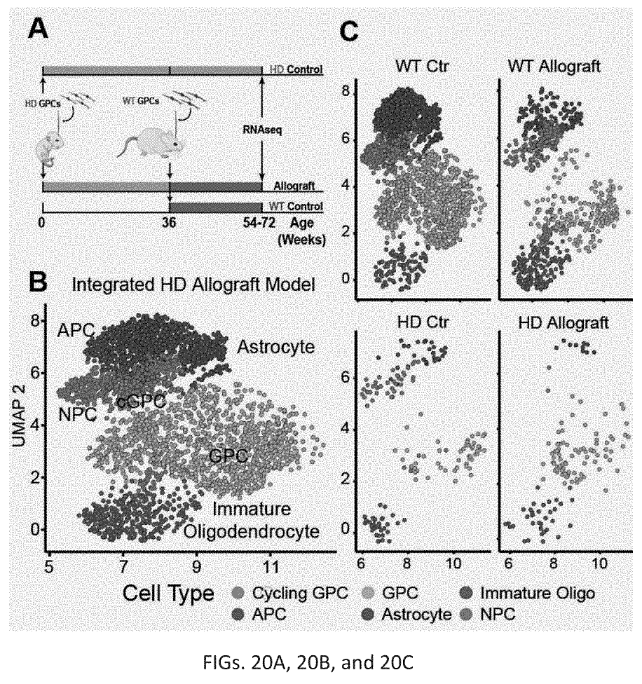

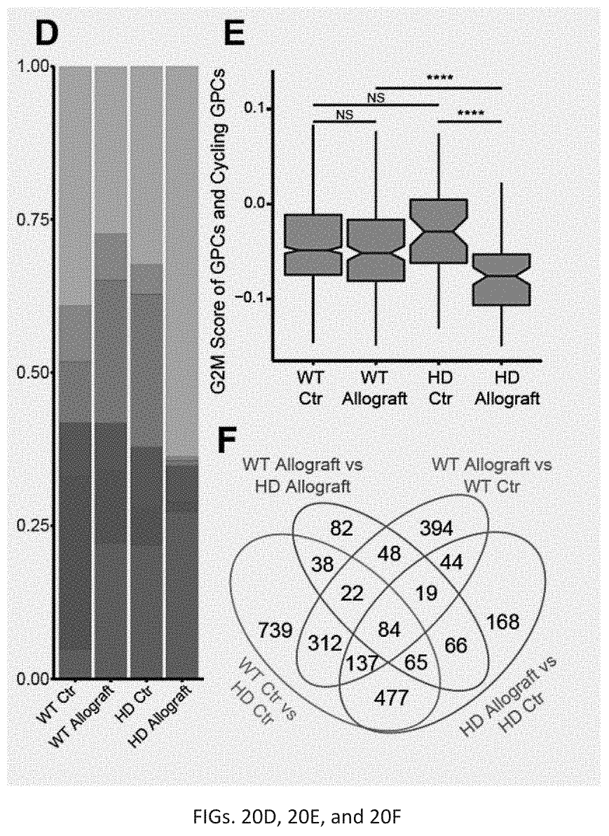

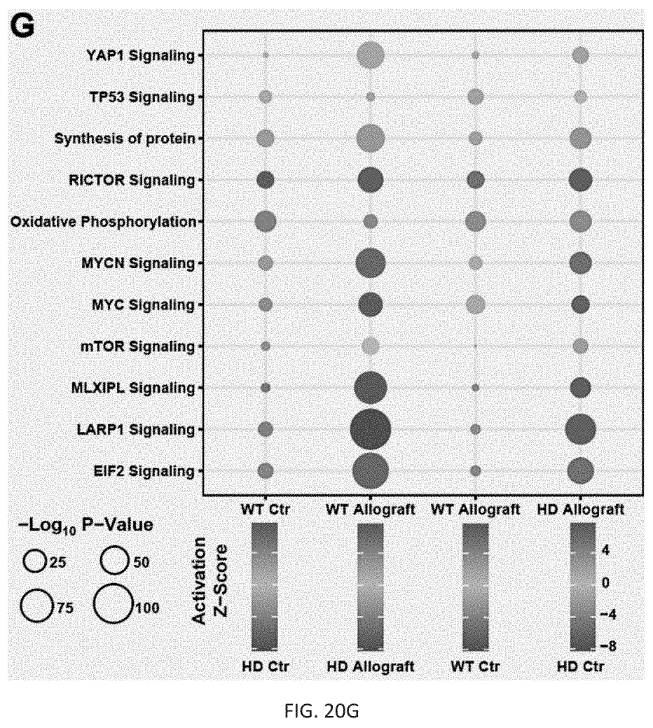

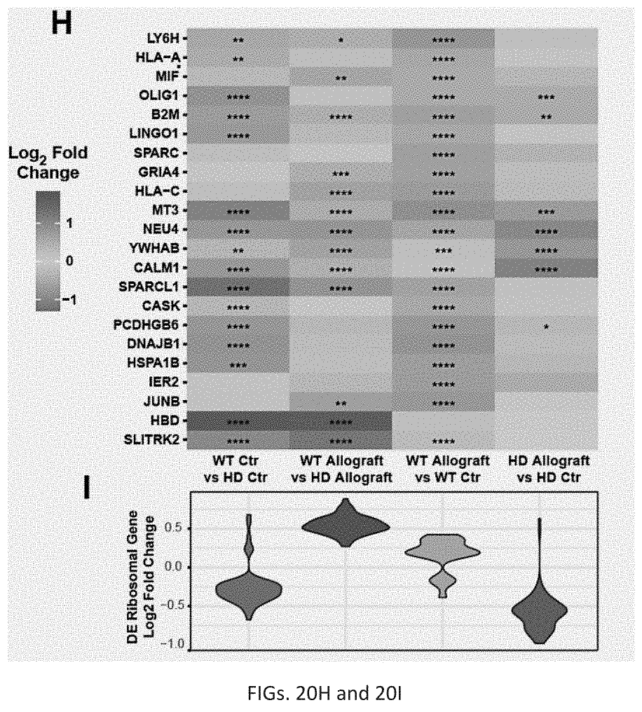

These observations suggest that this process was driven by a recapitulation of developmental cell competition, an evolutionarily conserved selection process by which less fit clones are sensed and eliminated from a tissue by their fitter neighbors, but as manifested here dynamically in the adult brain. This process has been shown in a variety of systems to comprise the active elimination of relatively slowly growing cells by their faster growing, more competitively fit neighbors. It was noted that in the adult brain, WT hGPCs typically expanded from their implantation sites in an advancing proliferative wave. These younger hGPCs largely eliminated their hitherto stably resident—and hence older—counterparts, whether the latter were mHTT-expressing HD cells, or isogenic WT cells that had been transplanted months earlier. In both cases, the younger cells ultimately recolonized their host brains with healthy new hGPCs (FIGS. 4 and 17), and in both cases the younger donor cells differentially expressed gene sets associated with competitive dominance (FIGS. 20-22). In particular, the competitive dominance of younger, adult-transplanted hGPCs was associated with their increased levels of predicted MYC and YAP1 pathway activity. These data provided a striking parallel to cell-cell competition in the mouse embryo, in which defective cells are eliminated by their neighbors following the acquisition of differential MYC expression during competitive challenge, and in which YAP and MYC interact to determine competitive outcomes during cell-cell competition. Indeed, the concurrent enrichment for YAP1 pathway members in “winner” WT hGPCs, including transcripts both upstream and downstream of YAP1, suggests that the Hippo pathway might be an especially promising target for the regulation of glial replacement in the adult human brain. Indeed, these observations parallel the results of liver repopulation studies, in which mouse fetal liver progenitors were found to drive faster and more extensive replacement when allografted into older than into younger hosts, and for which MYC and YAP1 activities were predominant determinants of competitive success. As such, the identification of YAP1 and MYC as important regulators of competition among hGPCs may enable strategies by which to further enhance the competitive advantage, speed and extent of donor cell colonization following the delivery of these cells to the brain.

The competitive replacement of resident glia by younger hGPCs that were observed resembles that of mouse glial replacement by implanted human GPCs, as their expansion within the murine brain is also sustained by a relative proliferative advantage, and progresses with the elimination of their murine counterparts upon contact. As in the xenograft setting, the winning population of young WT hGPCs appears to trigger the apoptotic death and local elimination of the resident losing population, whether comprised of older isogenic WT or sibling HD cells. The relative localization of dying host cells to the advancing wavefronts of younger WT cells suggests that the latter trigger the death of already-resident hGPCs via contact-dependent means. Potential mechanisms for such contact-dependent expression of relative cell fitness have been described in a variety of models, and include selective expression of Fwr isoforms, as well as mechanical signals, potentially transduced through Piezo1-dependent modulation of YAP. In addition, the selective elimination of both HD and isogenic hGPCs when confronted with younger hGPCs was paralleled by their depletion of ribosomal encoding transcripts, consistent with the loss of ribosomal transcripts by ‘loser’ cells during cell competition, and highlighting the contribution of ribosomal protein transcription to the regulation of cell fitness. Together, these data suggest that the transcriptional control of translational machinery is as important in cell-cell competition in the adult brain as it is in development.

These observations suggest that the brain may be a far more dynamic structural environment than previously recognized, with cell-cell competition among glial progenitor cells—and potentially their derived astrocytes—playing as critical a role in adult brain maintenance as in development. Indeed, this competitive advantage inventors noted of young over older resident cells seems to largely mimic development, where successive waves of GPCs compete amongst each other, with the oldest largely eradicated from the brain by birth, replaced by younger successors. In adulthood, one may similarly envision that somatic mutation among dividing glial progenitors may yield selective clonal advantage to one daughter lineage or the other, resulting in the inexorable competitive replacement of the population by descendants of the dominant daughter. This scenario, while typifying the onset of carcinogenesis and potentially gliomagenesis as well, may also be involved in tumor suppression, via the competitive elimination of neoplastic cells by more fit non-neoplastic neighbors. It is especially intriguing to consider whether such a process of dynamic competition among differentially fit hGPCs may be similarly involved in the development of non-neoplastic adult-onset brain disorders in which glia are involved, such as some schizophrenias, and HD itself. Indeed, such a mechanism may contribute to the late-stage acceleration in disease progression often noted among those neurodegenerative and neuropsychiatric disorders in which glial pathology is involved. In broad terms, these data suggest that resident, and hence older, diseased human glia may be replaced following the introduction of younger and healthier hGPCs. Indeed, such glial replacement may offer a viable strategy towards the cell-based treatment of those diseases of the human brain in which glial cells are causally involved.

Rejuvenation of Glial Progenitor Cells or Progenies Thereof

Some aspects of this disclosure relate to rejuvenation of glial progenitor cells or their progeny cells. Human glial progenitor cells emerge during the 2nd trimester to colonize the brain, in which a parenchymal pool remains throughout adulthood. While fetal hGPCs are highly migratory and proliferative, their expansion competence diminishes with age, as well as following demyelination-associated turnover.

As disclosed herein, to determine the basis for their decline in mobilization capacity, bulk and single cell RNA-Sequencing were used to compare the transcriptional programs of fetal and adult hGPCs. To that end, age-associated changes in gene expression were identified suggesting a loss of proliferative competence, concurrent with the onset of differentiation and senescence-associated transcriptional programs. More specifically, adult hGPCs developed a repressive transcription factor network centered on MYC, and regulated by ZNF274, MAX, IKZF3, and E2F6. Shown below are some exemplary nucleic acid sequences and amino acid sequences of these repressors.

E2F6
CDNA (SEQ ID NO: 4):
ATGAGTCAGCAGCGGCCGGCGAGGAAGTTACCCAGTCTCCTCCTGGACC
CGACGGAGGAGACGGTTCGCCGTCGGTGCCGAGACCCCATCAACGTGGA
GGGCCTGCTGCCATCAAAAATAAGGATTAATTTAGAAGATAATGTACAA
TATGTGTCCATGAGAAAAGCTCTAAAAGTGAAGAGACCTCGTTTTGATG
TATCGCTGGTTTATTTAACTCGAAAATTTATGGATCTTGTCAGATCTGC
TCCCGGGGGTATTCTTGACTTAAACAAGGTTGCAACGAAACTGGGAGTC
CGAAAGCGGAGAGTGTATGACATCACCAATGTCTTAGATGGAATCGACC
TCGTTGAAAAGAAATCCAAGAACCATATTAGATGGATAGGATCTGATCT
TAGCAATTTTGGAGCAGTTCCCCAACAAAAGAAGCTACAGGAGGAACTT
TCTGACTTATCAGCAATGGAAGATGCTTTGGATGAGTTAATTAAGGATT
GTGCTCAGCAGCTGTTTGAGTTAACAGATGACAAAGAAAATGAAAGACT
AGCATATGTGACCTATCAAGACATTCATAGCATTCAGGCCTTCCATGAA
CAGATCGTCATTGCAGTTAAAGCTCCAGCAGAAACCAGATTGGATGTTC
CAGCTCCCAGAGAAGACTCTATCACAGTGCACATAAGGAGCACCAACGG
ACCTATCGATGTCTATTTGTGTGAAGTGGAGCAGGGTCAGACCAGTAAC
AAAAGGTCTGAAGGTGTCGGGACCTCTTCATCTGAGAGCACTCATCCAG
AAGGCCCTGAGGAAGAAGAAAATCCTCAGCAAAGTGAAGAATTGCTTGA
AGTAAGCAACTGA
Amino Acid (SEQ ID NO: 5):
MSQQRPARKLPSLLLDPTEETVRRRCRDPINVEGLLPSKIRINLEDNVQ
YVSMRKALKVKRPRFDVSLVYLTRKFMDLVRSAPGGILDLNKVATKLGV
RKRRVYDITNVLDGIDLVEKKSKNHIRWIGSDLSNFGAVPQQKKLQEEL
SDLSAMEDALDELIKDCAQQLFELTDDKENERLAYVTYQDIHSIQAFHE
QIVIAVKAPAETRLDVPAPREDSITVHIRSTNGPIDVYLCEVEQGQTSN
KRSEGVGTSSSESTHPEGPEEEENPQQSEELLEVSN
IKZF3
CDNA (SEQ ID NO: 6):
ATGGGAAGTGAAAGAGCTCTCGTACTGGACAGATTAGCAAGCAATGTGG
CAAAACGAAAAAGCTCAATGCCTCAGAAATTCATTGGTGAGAAGCGCCA
CTGCTTTGATGTCAACTATAATTCAAGTTACATGTATGAGAAAGAGAGT
GAGCTCATACAGACCCGCATGATGGACCAAGCCATCAATAACGCCATCA
GCTATCTTGGCGCCGAAGCCCTGCGCCCCTTGGTCCAGACACCGCCTGC
TCCCACCTCGGAGATGGTTCCAGTTATCAGCAGCATGTATCCCATAGCC
CTCACCCGGGCTGAGATGTCAAACGGTGCCCCTCAAGAGCTGGAAAAGA
AAAGCATCCACCTTCCAGAGAAGAGCGTGCCTTCTGAGAGAGGCCTCTC
TCCCAACAATAGTGGCCACGACTCCACGGACACTGACAGCAACCATGAA
GAACGCCAGAATCACATCTATCAGCAAAATCACATGGTCCTGTCTCGGG
CCCGCAATGGGATGCCACTTCTGAAGGAGGTTCCCCGCTCTTACGAACT
CCTCAAGCCCCCGCCCATCTGCCCAAGAGACTCCGTCAAAGTGATCAAC
AAGGAAGGGGAGGTGATGGATGTGTATCGGTGTGACCACTGCCGCGTCC
TCTTCCTGGACTATGTGATGTTCACGATTCACATGGGCTGCCACGGCTT
CCGTGACCCTTTCGAGTGTAACATGTGTGGATATCGAAGCCATGATCGG
TATGAGTTCTCGTCTCACATAGCCAGAGGAGAACACAGAGCCCTGCTGA
AGTGA
Amino Acid (SEQ ID NO: 7):
MGSERALVLDRLASNVAKRKSSMPQKFIGEKRHCFDVNYNSSYMYEKES
ELIQTRMMDQAINNAISYLGAEALRPLVQTPPAPTSEMVPVISSMYPIA
LTRAEMSNGAPQELEKKSIHLPEKSVPSERGLSPNNSGHDSTDTDSNHE
ERQNHIYQQNHMVLSRARNGMPLLKEVPRSYELLKPPPICPRDSVKVIN
KEGEVMDVYRCDHCRVLFLDYVMFTIHMGCHGFRDPFECNMCGYRSHDR
YEFSSHIARGEHRALLKMAX
MAX
CDNA (SEQ ID NO: 8):
ATGAGCGATAACGATGACATCGAGGTGGAGAGCGACGAAGAGCAACCGA
GGTTTCAATCTGCGGCTGACAAACGGGCTCATCATAATGCACTGGAACG
AAAACGTAGGGACCACATCAAAGACAGCTTTCACAGTTTGCGGGACTCA
GTCCCATCACTCCAAGGAGAGAAGCTCTATTTCCTCTTTTGGAAATTGT
GTACTCCTGTCCTTCATCGTCAAAGTTTGATGCAGAAATGCCACACCTT
CATTTCAAGCTACCAAGTGCACAAGAAAAAAGAATGCAAGATTTAA
Amino Acid (SEQ ID NO: 9):
MSDNDDIEVESDEEQPRFQSAADKRAHHNALERKRRDHIKDSFHSLRDS
VPSLQGEKLYFLFWKLCTPVLHRQSLMQKCHTFISSYQVHKKKECKI
ZNF274
CDNA (SEQ ID NO: 10):
ATGCTGGAGAACTACAGGAACCTGGTCTCAGTGGAACATCAGCTTTCCAA
ACCAGATGTGGTATCTCAGTTAGAGGAGGCAGAAGATTTCTGGCCAGTGG
AGAGAGGAATTCCTCAAGACACCATTCCAGAGTATCCTGAGCTCCAGCTG
GACCCTAAATTGGATCCTCTTCCTGCTGAGAGTCCCCTAATGAACATTGA
GGTTGTTGAGGTCCTCACACTGAACCAGGAGGTGGCTGGTCCCCGGAATG
CCCAGATCCAGGCCCTATATGCTGAAGATGGAAGCCTGAGTGCAGATGCC
CCCAGTGAGCAGGTCCAACAGCAGGGCAAGCATCCAGGTGACCCTGAGGC
CGCGCGCCAGAGGTTCCGGCAGTTCCGTTATAAGGACATGACAGGTCCCC
GGGAGGCCCTGGACCAGCTCCGAGAGCTGTGTCACCAGTGGCTACAGCCT
AAGGCACGCTCCAAGGAGCAGATCCTGGAGCTGCTGGTGCTGGAGCAGTT
CCTAGGTGCACTGCCTGTGAAGCTCCGGACATGGGTGGAATCGCAGCACC
CAGAGAACTGCCAAGAGGTGGTGGCCCTGGTAGAGGGTGTGACCTGGATG
TCTGAGGAGGAAGTACTTCCTGCAGGACAACCTGCCGAGGGCACCACCTG
CTGCCTCGAGGTCACTGCCCAGCAGGAGGAGAAGCAGGAGGATGCAGCCA
TCTGCCCAGTGACAGTGCTCCCTGAGGAGCCAGTGACCTTCCAGGATGTG
GCTGTGGACTTCAGCCGGGAGGAGTGGGGGCTGCTGGGCCCGACACAGAG
GACCGAGTACCGCGATGTGATGCTGGAGACCTTTGGGCACCTGGTCTCTG
TGGGGTGGGAGACTACACTGGAAAATAAAGAGTTAGCTCCAAATTCTGAC
ATTCCTGAGGAAGAACCAGCCCCCAGCCTGAAAGTACAAGAATCCTCAAG
GGATTGTGCCTTGTCCTCTACATTAGAAGATACCTTGCAGGGTGGGGTCC
AGGAAGTCCAAGACACAGTGTTGAAGCAGATGGAGTCTGCTCAGGAAAAA
GACCTTCCTCAGAAGAAGCACTTTGACAACCGTGAGTCCCAGGCAAACAG
TGGTGCTCTTGACACAAACCAAGTTTCGCTCCAGAAAATTGACAACCCTG
AGTCCCAGGCAAACAGTGGCGCTCTTGACACAAACCAAGTTTTGCTCCAC
AAAATTCCTCCTAGAAAACGATTGCGCAAACGTGACTCACAAGTTAAAAG
TATGAAACATAATTCACGTGTAAAAATTCATCAGAAGAGCTGTGAAAGGC
AAAAGGCCAAGGAAGGCAATGGTTGTAGGAAAACCTTCAGTCGGAGTACT
AAACAGATTACGTTTATAAGAATTCACAAGGGGAGCCAAGTTTGCCGATG
CAGTGAATGTGGTAAAATATTCCGGAACCCAAGATACTTTTCTGTGCATA
AGAAAATCCATACCGGAGAGAGGCCCTATGTGTGTCAAGACTGTGGGAAA
GGATTTGTTCAGAGCTCTTCCCTCACACAGCATCAGAGAGTTCATTCTGG
AGAGAGACCATTTGAATGTCAGGAGTGTGGGAGGACCTTCAATGATCGCT
CAGCCATCTCCCAGCACCTGAGGACTCACACTGGCGCTAAGCCCTACAAG
TGTCAGGACTGTGGAAAAGCCTTCCGCCAGAGCTCCCACCTCATCAGACA
TCAGAGGACTCACACCGGGGAGCGCCCATATGCATGCAACAAATGTGGAA
AGGCCTTCACCCAGAGCTCACACCTTATTGGGCACCAGAGAACCCACAAT
AGGACAAAGCGAAAGAAGAAACAGCCTACCTCATAG
Amino Acid (SEQ ID NO: 11):
MLENYRNLVSVEHQLSKPDVVSQLEEAEDFWPVERGIPQDTIPEYPELQ
LDPKLDPLPAESPLMNIEVVEVLTLNQEVAGPRNAQIQALYAEDGSLSA
DAPSEQVQQQGKHPGDPEAARQRFRQFRYKDMTGPREALDQLRELCHQW
LQPKARSKEQILELLVLEQFLGALPVKLRTWVESQHPENCQEVVALVEG
VTWMSEEEVLPAGQPAEGTTCCLEVTAQQEEKQEDAAICPVTVLPEEPV
TFQDVAVDFSREEWGLLGPTQRTEYRDVMLETFGHLVSVGWETTLENKE
LAPNSDIPEEEPAPSLKVQESSRDCALSSTLEDTLQGGVQEVQDTVLKQ
MESAQEKDLPQKKHFDNRESQANSGALDTNQVSLQKIDNPESQANSGAL
DTNQVLLHKIPPRKRLRKRDSQVKSMKHNSRVKIHQKSCERQKAKEGNG
CRKTFSRSTKQITFIRIHKGSQVCRCSECGKIFRNPRYFSVHKKIHTGE
RPYVCQDCGKGFVQSSSLTQHQRVHSGERPFECQECGRTFNDRSAISQH
LRTHTGAKPYKCQDCGKAFRQSSHLIRHQRTHTGERPYACNKCGKAFTQ
SSHLIGHQRTHNRTKRKKKQPTS

Individual over-expression of each of these factors in human iPSC-derived GPCs led to a loss of proliferative gene expression and an induction of markers of senescence, that replicated the transcriptional changes incurred during glial aging. Parallel miRNA profiling identified an adult-selective miRNA expression signature, whose targets may further constrain the expansion competence of aged GPCs. These observations indicate that hGPCs 20 age through the acquisition of a MYC-repressive environment, suggesting that suppression of these repressors of glial expansion and turnover permits the effective rejuvenation of aged hGPCs.

Glial progenitor cells (GPCs, also referred to as oligodendrocyte progenitor cells and NG2 cells) colonize the human brain during development, and persist in abundance throughout adulthood. During development, human GPCs (hGPCs) are highly proliferative bipotential cells, producing new oligodendrocytes and astrocytes (Ffrench-Constant and Raff, 1986; Raff et al., 1983). In rodents, this capacity wanes during normal aging, with proliferation, migration, and differentiation competence all diminishing in aged GPCs (Chari et al., 2003; Gao and Raff, 1997; Moyon et al., 2021; Segel et al., 2019; Tang et al., 2000; Temple and Raff, 1986; Wolswijk and Noble, 1989; Wren et al., 1992). Similarly, adult human GPCs are less proliferative, less migratory, and more readily differentiated than their fetal counterparts when transplanted into congenitally dysmyelinated murine hosts (Windrem et al., 2004). Yet despite the manifestly different competencies of fetal and adult hGPCs, and the abundant data on GPC transcription in rodent models of aging, little data are available that address changes in GPC gene expression during human aging (Perlman et al., 2020; Sim et al., 2006), or that provide clear head-to-head comparisons of transcription by fetal and adult human GPCs. Certain parts of this disclosure therefore compare the transcriptional patterns of fetal and adult hGPCs, and use that data to identify those regulatory pathways causally linked to the maturation and aging of these cells

To this end, inventors first utilized bulk and single cell RNA-Sequencing (scRNA-Seq) of A2B5₊ and CD140a/PDGFRα₊ hGPCs isolated from human fetal forebrain, so as to define their transcriptional signatures and heterogeneity. Inventors then compared these data to the gene expression of isolated adult hGPCs, and found that the latter exhibited transcriptional patterns suggesting a loss of proliferative capacity, the onset of an early phenotypically-differentiated profile, and the induction of senescence. Transcription factor motif enrichment analysis of the promoters of differentially expressed genes then implicated the adult-induced transcriptional repressors E2F6, ZNF274, MAX, and IKZF3 as principal drivers of the human glial aging program. Network analysis strongly suggested that as a group, these genes worked though the inhibition of MYC and its proximal targets, which were relatively over-expressed in fetal hGPCs. Critically, it was then found that over-expression of these adult repressors in newly generated human iPSC-derived GPCs, which are analogous to fetal hGPCs in their expression signatures, indeed led to the induction of transcriptional signatures that substantially recapitulated those of adult GPCs. Inventors then identified a cohort of miRNAs selectively-expressed by adult hGPCs, that were predicted to post-transcriptionally inhibit fetal GPC gene expression, especially so in concert with the adult-acquired repressor network. Together, these data suggest that a cohort of repressors appears during the aging of adult human GPCs, whose activity is centered on MYC and MYC-dependent transcription. As such, these repressors may comprise feasible therapeutic targets, whose modulation may restore salient features of the mitotic and differentiation competence of aged or otherwise mitotically-exhausted GPCs.

Suppressor/Rejuvenation Therapy

In one aspect, the present disclosure provides therapy methods by suppressing a transcription repressor selected from the group consisting of E2F6, ZNF274, MAX, and IKZF3. In some examples, this can be achieved by administering to a subject in need thereof or a target cell in need thereof a suppressor or inhibitor of one or more of the transcription repressor. Such a suppressor or inhibitor can comprise or be a small molecule compound, an oligonucleotide, a nucleic acid, a peptide, a polypeptide, a CRISPR/Cas system, or an antibody or an antigen-binding portion thereof. Examples of the suppressor/inhibitor include activators, agonists, or potentiators of the related YAP or MYC pathway signaling pathways (e.g., the Hippo signaling pathway). Various activators for this signaling pathway are known in the art. In some embodiments, the suppressor is an inhibitory nucleic acid or interfering nucleic acid, such as siRNA, shRNA, miRNA, antisense oligonucleotides (ASOs), and/or a nucleic acid comprising one or more modified nucleic acid residues.

Inhibitory Nucleic Acids

Certain aspects of the disclosure provide one or more inhibitory nucleic acids (e.g., inhibitory RNA molecules), polynucleotides encoding such inhibitory nucleic acids, and transgenes engineered to express such inhibitory nucleic acids. The one or more inhibitory nucleic acids may target the same gene (e.g., hybridize or specifically bind to a same mRNA sequence or different mRNA sequences of the same gene) or different genes (e.g., hybridize or specifically bind to mRNAs of different genes). Accordingly, the methods described herein can include reducing expression of E2F6, ZNF274, MAX, or IKZF3 gene using inhibitory nucleic acids that target the E2F6, ZNF274, MAX, or IKZF3 gene or mRNA

An inhibitory nucleic acid refers to a nucleic acid that can bind to a target nucleic acid (e.g., a target RNA) in a cell and reduce or inhibit the level or function of the target nucleic acid in the cell. Example of the inhibitory nucleic acid include antisense oligonucleotides, ribozymes, external guide sequence (EGS) oligonucleotides, small interfering (si)RNA compounds, single- or double-stranded RNA interference compounds, modified bases/locked nucleic acids (LNAs), antagomirs, peptide nucleic acids (PNAs), and other oligomeric compounds or oligonucleotide mimetics that specifically hybridize to at least a portion of a target nucleic acid (e.g., E2F6, ZNF274, MAX, or IKZF3 mRNA) and modulate its level or function.

In some embodiments, the inhibitory nucleic acid can be an antisense RNA, an antisense DNA, a chimeric antisense oligonucleotide, an antisense oligonucleotide comprising modified linkages, an interference RNA (iRNA), a short or small interfering RNA (siRNA), a micro RNA or micro interfering RNA (miRNA), a small temporal RNA (stRNA), a short hairpin RNA (shRNA), a small RNA-induced gene activation agent (RNAa), a small activating RNA (saRNA), or combinations thereof. The inhibitory nucleic acids can be modified, e.g., to include a modified nucleotide (e.g., locked nucleic acid) or backbone (e.g., backbones that do not include a phosphorus atom therein), or can by mixmers or gapmers; see, e.g., WO2013/006619, which is incorporated herein by reference for its teachings related to modifications of oligonucleotides.

In some examples, the inhibitory nucleic acid is an inhibitory RNA molecule that mediates RNA interference (RNAi), a process by which cells regulate gene expression. A double-stranded RNA (dsRNA) in the cell cytoplasm triggers the RNAi pathway in which the double-stranded RNA is processed into small double-stranded fragments of approximately 21-23 nucleotides in length by the RNAse III-like enzyme DICER. These double-stranded fragments are integrated into a multi-subunit protein called the RNA-induced silencing complex (RISC). The RISC contains Argonaute proteins that unwind the double-stranded fragment into a passenger strand that is removed from the complex and a guide strand that is complementary to a target sequence in a specific mRNA and which directs the RISC complex to cleave or suppress the translation of the specific target mRNA molecule (Kotowska-Zimmer et al., 2021). In this way the gene that encoded the mRNA molecule is rendered essentially inactive or “silenced.”

RNAi technology may employ a number of tools, including synthetic siRNAs, vector-based shRNAs, and artificial miRNAs (amiRNAs). Synthetic siRNAs are exogenous double stranded RNAs that must be delivered into cells and must overcome stability and pharmacokinetic challenges. shRNAs are artificial RNA molecules with a tight hairpin loop structure that are delivered to cells using plasmids or viral expression vectors. shRNAs are typically transcribed from strong pol III promoters (e.g., U6 or H1) and enter the RNAi pathway as hairpins. However, transcription driven by strong pol III promoters can produce supraphysiologic levels of shRNA that saturate the endogenous miRNA biogenesis machinery, resulting in toxicity. AmiRNAs embed a target-specific shRNA insert in a scaffold based on a natural primary miRNA (pri-miRNA). This ensures proper processing and transport similar to endogenous miRNAs, resulting in lower toxicity (Kotowska-Zimmer et al., 2021).

In some embodiments of this disclosure, the inhibitory RNA molecule can be an siRNA, a miRNA (including an amiRNA), or an shRNA. An siRNA is known in the art as a double-stranded RNA molecule of approximately 19-25 (e.g., 19-23) base pairs in length that induces RNAi in a cell. In some embodiments, the siRNA sequence can also be inserted into an artificial miRNA scaffold (“shmiRNA”). An shRNA is known in the art as an RNA molecule comprising approximately 19-25 (e.g., 19-23) base pairs of double stranded RNA linked by a short loop (e.g., about 4-11 nucleotides) that induces RNAi in a cell. An miRNA is known in the art as an RNA molecule that induces RNAi in a cell comprising a short (e.g., 19-25 base pairs) sequence of double-stranded RNA linked by a loop and containing one or more additional sequences of double-stranded RNA comprising one or more bulges (e.g., mis-paired or unpaired base pairs).

As used herein, the term “miRNA” encompasses endogenous miRNAs as well as exogenous or heterologous miRNAs. In some embodiments, “miRNA” may refer to a pri-miRNA or a pre-miRNA. During miRNA processing, a pri-miRNA transcript is produced. The pri-miRNA is processed by Drosha-DGCR8 to produce a pre-miRNA by excising one or more sequences to leave a pre-miRNA with a 5′ flanking region, a guide strand, a loop region, a non-guide strand, and a 3′ flanking region; or a 5′ flanking region, a non-guide strand, a loop region, a guide strand, and a 3′ flanking region. The pre-miRNA is then exported to the cytoplasm and processed by Dicer to yield a siRNA with a guide strand and a non-guide (or passenger) strand. The guide strand is then used by the RISC complex to catalyze gene silencing, e.g., by recognizing a target RNA sequence complementary to the guide strand. Further description of miRNAs may be found, e.g., in WO 2008/150897. The recognition of a target sequence by a miRNA is primarily determined by pairing between the target and the miRNA seed sequence, e.g., nucleotides 1-8 (5′ to 3′) of the guide strand (see, e.g., Boudreau, R. L. et al. (2013) Nucleic Acids Res. 41:e9).

Shown below are some exemplary suppressor miRNAs that target and suppress one or more of E2F6, ZNF274, MAX, and IKZF3.

TABLE
Sequences of suppressor miRNAs

| Name | Nucleic Acid Sequence | Accession number | SEQ ID NO |
|---|---|---|---|
| hsa-miR-125b-5p | ucccugagacccuaacuuguga | MIMAT0000423 | 12 |
| hsa-miR-106a-5p | aaaagugcuuacagugcagguag | MIMAT0000103 | 13 |
| hsa-miR-17-5p | caaagugcuuacagugcagguag | MIMAT0000070 | 14 |
| hsa-miR-130a-3p | cagugcaauguuaaaagggcau | MIMAT0000425 | 15 |
| hsa-miR-130b-3p | cagugcaaugaugaaagggcau | MIMAT0000691 | 16 |
| hsa-miR-379-5p | ugguagacuauggaacquagg | MIMAT0000733 | 17 |
| hsa-miR-93-3p | acugcugagcuagcacuucccg | MIMAT0004509 | 18 |
| hsa-miR-1260b | aucccaccacugccaccau | MIMAT0015041 | 19 |
| hsa-miR-767-5p | ugcaccaugguugucugagcaug | MIMAT0003882 | 20 |
| hsa-miR-30b-5p | uguaaacauccuacacucagcu | MIMAT0000420 | 21 |
| hsa-miR-9-3p | auaaagcuagauaaccgaaagu | MIMAT0000442 | 22 |
| hsa-miR-9-5p | ucuuugguuaucuagcuguauga | MIMAT0000441 | 23 |
| hsa-miR-485-5p | agaggcuggccgugaugaauuc | MIMAT0002175 | 24 |

In some embodiments of this disclosure, an inhibitory RNA molecule forms a hairpin structure. Generally, hairpin-forming RNAs are arranged into a self-complementary “stem-loop” structure that includes a single nucleic acid encoding a stem portion having a duplex comprising a sense strand (e.g., passenger strand) connected to an antisense strand (e.g., guide strand) by a loop sequence. The passenger strand and the guide strand share complementarity. In some embodiments, the passenger strand and guide strand share 100% complementarity. In some embodiments, the passenger strand and guide strand share at least 50%, at least 60%, at least 70%, at least 80%, at least 90%, at least 95%, or at least 99% complementarity. A passenger strand and a guide strand may lack complementarity due to a base-pair mismatch. In some embodiments, the passenger strand and guide strand of a hairpin-forming RNA may have at least 1, at least 2, at least 3, at least 4, at least 5, at least 6, at least 7 at least 8, at least 9, or at least 10 mismatches. Generally, the first 2-8 nucleotides of the stem (relative to the loop) are referred to as “seed” residues and play an important role in target recognition and binding. The first residue of the stem (relative to the loop) is referred to as the “anchor” residue. In some embodiments, hairpin-forming RNA have a mismatch at the anchor residue.

In some embodiments, an inhibitory RNA molecule is processed in a cell (or subject) to form a “mature miRNA”. Mature miRNA is the result of a multistep pathway which is initiated through the transcription of primary miRNA from its miRNA gene or intron, by RNA polymerase II or III generating the initial precursor molecule in the biological pathway resulting in miRNA. Once transcribed, pri-miRNA (often over a thousand nucleotides long with a hairpin structure) is processed by the Drosha enzyme which cleaves pri-miRNA near the junction between the hairpin structure and the ssRNA, resulting in precursor miRNA (pre-miRNA). The pre-miRNA is exported to the cytoplasm where is further reduced by Dicer enzyme at the pre-miRNA loop, resulting in duplexed miRNA strands.

Of the two strands of a miRNA duplex, one arm, the guide strand (miR), is typically found in higher concentrations and binds and associates with the Argonaute protein which is eventually loaded into the RNA-inducing silencing complex. The guide strand miRNA-RISC complex helps regulates gene expression by binding to its complementary sequence of mRNA, often in the 3′ UTR of the mRNA. The non-guide strand of the miRNA duplex is known as the passenger strand and is often degraded, but may persist and also act either intact or after partial degradation to have a functional role in gene expression.

In some embodiments, a transgene is engineered to express an inhibitory nucleic acid (e.g., an miRNA) having a guide strand that targets a human gene. “Targeting” refers to hybridization or specific binding of an inhibitory nucleic acid to its cognate (e.g., complementary) sequence on a target gene (e.g., mRNA transcript of a target gene). In some embodiments, an inhibitory nucleic acid that targets a gene transcript shares a region of complementarity with the target gene that is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some embodiments, a region of complementarity is more than 30 nucleotides in length.

Typically, the guide strand may target a human gene transcript associated with a disease or disorder of myelin. Examples include that for ZNF274, MAX, IKZF3, or E2F6. In some embodiments, a guide strand that targets any of these gene transcripts is encoded by an isolated nucleic acid comprising a suitable segment of the sequences set forth above.

Accordingly, the inhibitory nucleic acids can be used to mediate gene silencing, specifically one or more of ZNF274, MAX, IKZF3, and E2F6, via interaction with RNA transcripts or alternately by interaction with particular gene sequences, wherein such interaction results in gene silencing either at the transcriptional level or post-transcriptional level such as, for example, but not limited to, RNAi or through cellular processes that modulate the chromatin structure or methylation patterns of the target and prevent transcription of the target gene, with the nucleotide sequence of the target thereby mediating silencing.

These inhibitory nucleic acids can comprise short double-stranded regions of RNA. The double stranded RNA molecules can comprise two distinct and separate strands that can be symmetric or asymmetric and are complementary, i.e., two single-stranded RNA molecules, or can comprise one single-stranded molecule in which two complementary portions, e.g., a sense region and an antisense region, are base-paired, and are covalently linked by one or more single-stranded “hairpin” areas (i.e. loops) resulting in, for example, a single-stranded short-hairpin polynucleotide or a circular single-stranded polynucleotide.

The linker can be polynucleotide linker or a non-nucleotide linker. In some embodiments, the linker is a non-nucleotide linker. In some embodiments, a hairpin or circular inhibitory nucleic acid molecule contains one or more loop motifs, wherein at least one of the loop portion of the molecule is biodegradable. For example, a single-stranded hairpin molecule can be designed such that degradation of the loop portion of the molecule in vivo can generate a double-stranded siRNA molecule with 3′-terminal overhangs, such as 3′-terminal nucleotide overhangs comprising 1, 2, 3 or 4 nucleotides. Or alternatively, a circular inhibitory nucleic acid molecule can be designed such that degradation of the loop portions of the molecule in vivo can generate, for example, a double-stranded siRNA molecule, with 3′-terminal overhangs, such as 3′-terminal nucleotide overhangs comprising about 2 nucleotides.

In symmetric inhibitory nucleic acid molecules, each strand, the sense (passenger) strand and antisense (guide) strand, can be independently about 15 to about 40 (e.g., about 15, 16, 17, 18, 19, 20, 21, 22, 23, 24, 25, 26, 27, 28, 29, 30, 31, 32, 33, 34, 35, 36, 37, 38, 39, or 40) nucleotides in length

In asymmetric inhibitory nucleic acid molecules, the antisense region or strand of the molecule can be about 15 to about 30 (e.g., about 15, 16, 17, 18, 19, 20, 21, 22, 23, 24, 25, 26, 27, 28, 29, or 30) nucleotides in length, wherein the sense region is about 3 to about 25 (e.g., about 3, 4, 5, 6, 7, 8, 9, 10, 11, 12, 13, 14, 15, 16, 17, 18, 19, 20, 21, 22, 23, 24, or 25) nucleotides in length.

In yet other embodiments, inhibitory nucleic acid molecules described herein can comprise single stranded hairpin siRNA molecules, wherein the molecules can be about 25 to about 70 (e.g., about 25, 26, 27, 28, 29, 30, 31, 32, 33, 34, 35, 36, 40, 45, 50, 55, 60, 65, or 70) nucleotides in length.

In still other embodiments, the molecules may comprise single-stranded circular siRNA molecules, wherein the molecules are about 38 to about 70 (e.g., about 38, 40, 45, 50, 55, 60, 65, or 70) nucleotides in length.

In various symmetric embodiments, the inhibitory nucleic acid duplexes described herein independently may comprise about 15 to about 40 base pairs (e.g., about 15, 16, 17, 18, 19, 20, 21, 22, 23, 24, 25, 26, 27, 28, 29, 30, 31, 32, 33, 34, 35, 36, 37, 38, 39, or 40).

In yet other embodiments, where the inhibitory nucleic acid molecules described herein are asymmetric, the molecules may comprise about 3 to 25 (e.g., about 3, 4, 5, 6, 7, 8, 9, 10, 11, 12, 13, 14, 15, 16, 17, 18, 19, 20, 21, 22, 23, 24, or 25) base pairs).

In still other embodiments, where the inhibitory nucleic acid molecules are hairpin or circular structures, the molecules can comprise about 3 to about 30 (e.g., about 15, 16, 17, 18, 19, 20, 21, 22, 23, 24, 25, 26, 27, 28, 29, or 30) base pairs.

The sense strand and antisense strands or sense region and antisense regions of the inhibitory nucleic acid molecules can be complementary. Also, the antisense strand or antisense region can be complementary to a nucleotide sequence or a portion thereof of a target RNA (e.g., that of ZNF274, MAX, IKZF3, and E2F6). The sense strand or sense region if the inhibitory nucleic acid can comprise a nucleotide sequence of the target gene or a portion thereof.

In some embodiments, the inhibitory nucleic acid can be optimized (based on sequence) or chemically modified to minimize degradation prior to and/or upon delivery to the tissue of interest. Commercially available sources for these interfering nucleic acids include, but are not limited to, Thermo-Fisher Scientific/Ambion, Origene, Qiagen, Dharmacon, and Santa Cruz Biotechnology. In some embodiments, such optimizations and/or modifications may be made to assure sufficient payload of the inhibitory nucleic acid is delivered to the tissue of interest. Other embodiments include the use of small molecules, aptamers, or oligonucleotides designed to decrease the expression of a E2F6, ZNF274, MAX, or IKZF3 gene by either binding to a gene's DNA to limit expression, e.g., antisense oligonucleotides, or impose post-transcriptional gene silencing (PTGS) through mechanisms that include, but are not limited to, binding directly to the targeted transcript or gene product or one or more other proteins in such a way that said gene's expression is reduced; or the use of other small molecule decoys that reduce the specific gene's expression.

Any inhibitory nucleic acid molecule or construct described herein can comprise one or more chemical modifications. Modifications can be used to improve in vitro or in vivo characteristics such as stability, activity, toxicity, immune response (e.g., prevent stimulation of an interferon response, an inflammatory or pro-inflammatory cytokine response, or a Toll-like Receptor (TlF) response), and/or bioavailability.

Chemically modified molecules exhibit improved RNAi activity compared to corresponding unmodified or minimally modified molecules. The chemically modified motifs disclosed herein provide the capacity to maintain RNAi activity that is substantially similar to unmodified or minimally modified active siRNA while at the same time providing nuclease resistance and pharmacokinetic properties suitable for use in therapeutic applications.

In various embodiments, the inhibitory nucleic acid molecules described herein can comprise modifications wherein any (e.g., one or more or all) nucleotides present in the sense and/or antisense strand are modified nucleotides. In some embodiments, the molecules can be partially modified (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80 nucleotides are modified) with chemical modifications. In other embodiments, the molecules may be completely modified (e.g., 100% modified) with chemical modifications.

The chemical modification within a single molecule can be the same or different. In some embodiments, at least one strand has at least one chemical modification. In other embodiments, each strand has at least one chemical modifications, which can be the same or different, such as, sugar, base, or backbone (i.e., internucleotide linkage) modifications. In other embodiments, a molecules may contain at least 2, 3, 4, 5, or more different chemical modifications.

Non-limiting examples of suitable chemical modifications include those disclosed in, e.g., U.S. Pat. No. 8,202,979 and U.S. 20050266422 and include sugar, base, and phosphate, non-nucleotide modifications, and/or any combination thereof.

In various embodiments, a majority of the pyrimidine nucleotides present in the double-stranded inhibitory nucleic acid molecule comprises a sugar modification. In yet other embodiments, a majority of the purine nucleotides present in the double-stranded molecule comprises a sugar modification. In certain instances, the purines and pyrimidines are differentially modified at the 2′-sugar position (i.e., at least one purine has a different modification from at least one pyrimidine in the same or different strand at the 2′-sugar position).

In certain specific embodiments, at least one modified nucleotide is a 2′-deoxy-2-fluoro nucleotide, a 2′-deoxy nucleotide, or a 2′-O-alkyl (e.g., 2′-O-methyl) nucleotide. In yet other embodiments, at least one nucleotide has a ribo-like, Northern or A form helix configuration (see e.g., Saenger, Principles of Nucleic Acid Structure, Springer-Verlag ed., 1984). Non-limiting examples of nucleotides having a Northern configuration include locked nucleic acid (LNA) nucleotides (e.g., 2′-O, 4′-C-methylene-(D-ribofuranosyl) nucleotides); 2′-methoxyethoxy (MOE) nucleotides; 2′-methyl-thio-ethyl nucleotides, 2′-deoxy-2′-fluoro nucleotides, 2′-deoxy-2′-chloro nucleotides, 2′-azido nucleotides, 2′-O-trifluoromethyl nucleotides, 2′-O-ethyl-trifluoromethoxy nucleotides, 2′-O-difluoromethoxy-ethoxy nucleotides, 4′-thio nucleotides and 2′-O-methyl nucleotides.

The inhibitory nucleic acids described herein can be obtained using a number of techniques known to those of skill in the art. For example the inhibitory nucleic acids can be chemically synthesized or may be encoded by plasmid (e.g., transcribed as sequences that automatically fold into duplexes with hairpin loops). siRNA can also be generated by cleavage of longer dsRNA.

In some embodiments, inhibitory nucleic acids are chemically synthesized. Oligonucleotides (e.g., certain modified oligonucleotides or portions of oligonucleotides lacking ribonucleotides) can be synthesized using protocols known in the art, for example as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. The synthesis of oligonucleotides makes use of common nucleic acid protecting and coupling groups, such as dimethoxytrityl at the 5′-end, and phosphoramidites at the 3′-end.

Alternatively, the inhibitory nucleic acids can be synthesized separately and joined together post-synthetically, for example, by ligation (Moore et al., 1992, Science 256, 9923; Draper et al., International PCT Publication No. WO 93/23569; Shabarova et al., 1991, Nucleic Acids Research 19, 4247; Bellon et al., 1997, Nucleosides &Nucleotides, 16, 951; Bellon et al., 1997, Bioconjugate Chem. 8, 204), or by hybridization following synthesis and/or deprotection.

In some embodiments, inhibitory nucleic acids can be expressed and delivered from transcription units inserted into recombinant DNA or RNA vectors. The recombinant vectors can be DNA plasmids or viral vectors. Viral vectors can be constructed based on, but not limited to, adeno-associated virus, retrovirus, adenovirus, or alphavirus.

CRISPR/Cas System

In one aspect, suppressing or knocking down of one or more of the genes described herein can also be achieved via a CRISPR-Cas guided nuclease using a CRISPR/Cas system and related methods known in the art. See, e.g., U.S. Ser. No. 11/225,659B2, WO2021168799A1, WO2022188039A1, WO2022188797A1, WO2022068912A1, and WO2022047624A1. See also Gimenez et al., “CRISPR-on System for the Activation of the Endogenous human INS gene,” Gene Therapy 23: 543-547 (2016); Wiedenheft et al., “RNA-Guided Genetic Silencing Systems in Bacteria and Archaea,” Nature 482:331-338 (2012); Zhang et al., “Multiplex Genome Engineering Using CRISPR/Cas Systems,” Science 339(6121):819-23 (2013); and Gaj et al., “ZFN, TALEN, and CRISPR/Cas-based Methods for Genome Engineering,” Cell 31(7):397-405 (2013), which are hereby incorporated by reference in their entirety.

CRISPR-Cas system is a genetic technique which allows for sequence-specific control of gene expression in prokaryotic and eukaryotic cells by guided nuclease double-stranded DNA cleavage. It is based on the bacterial immune system-derived CRISPR (clustered regularly interspaced palindromic repeats) pathway.

In another aspect, this application provides a complex comprising: (i) a protein composition that comprise a Cas protein, or orthologs, homologs, derivatives, conjugates, functional fragments thereof, conjugates thereof, or fusions thereof; and (ii) a polynucleotide composition, comprising a CRISPR RNA and a programmable spacer sequence or guide sequence complementary to at least a portion of a target RNA or DNA. The programmable guide RNA, CRISPR RNA and the Cas protein together form a CRISPR/Cas-based module for sequence targeting and recognition.

The target RNA can be any RNA molecule of interest, including naturally-occurring and engineered RNA molecules. The target RNA can be an mRNA, a tRNA, a ribosomal RNA (rRNA), a microRNA (miRNA), an interfering RNA (siRNA), a ribozyme, a riboswitch, a satellite RNA, a microswitch, a microzyme, or a viral RNA.

In some embodiments, the target nucleic acid is associated with a condition or disease, such as a condition or disorder mediated by loss of while matter/oligodendrocytes/astrocytes and related disorders as described herein. Thus, in some embodiments, the systems described herein can be used to treat such a condition or disease by targeting these nucleic acids.

For instance, the target nucleic acid associated with a condition or disease may be an RNA molecule that is overexpressed in a diseased cell, an old or older cell, or a senescent cell. The target nucleic acid may also be a toxic RNA and/or a mutated RNA (e.g., an mRNA molecule having a splicing defect or a mutation). The target nucleic acid may also be an miRNA.

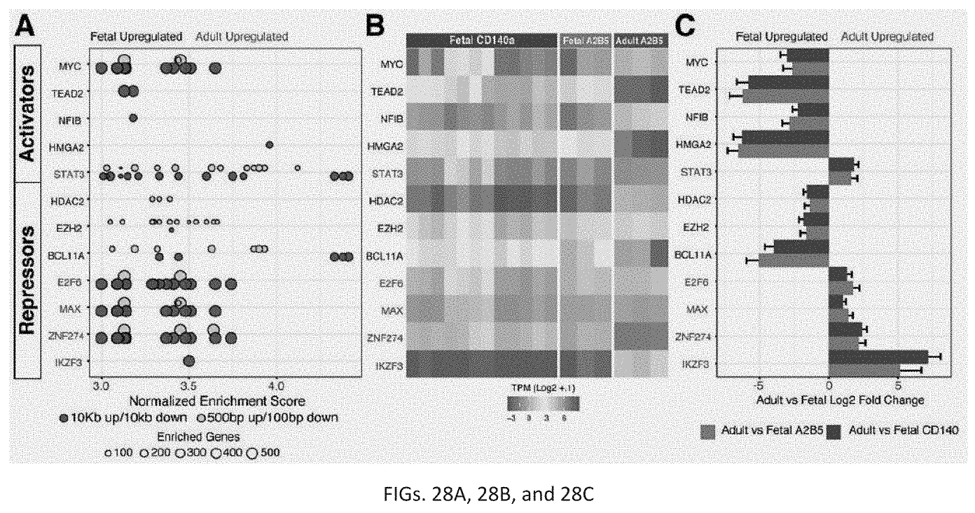

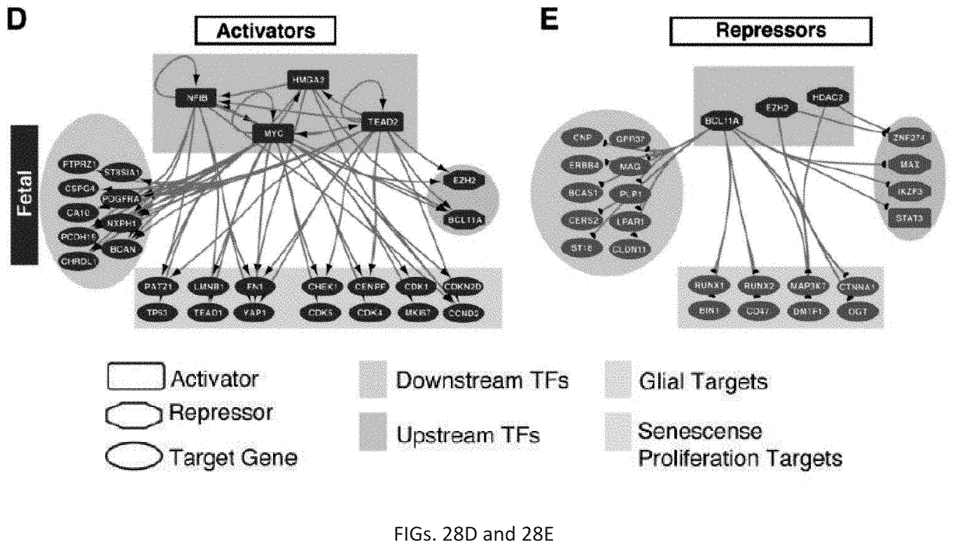

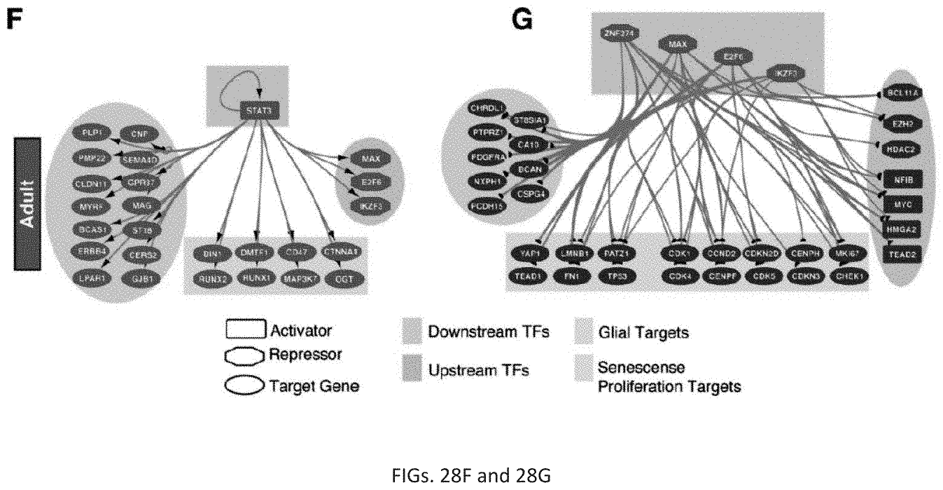

For example, the target nucleic acid may be that of a gene whose increased activity has been linked to senescence, such as STATs, and a transcription repressor (e.g., E2F6, ZNF274, MAX, or IKZF3) as illustrated FIGS. 28, 30, and 37. The target nucleic acid may be that of an miRNA that promotes senescence in adult GPCs, such as miR-584-5p, miR-330-3p, miR-23b-3p, and miR-140-3p as illustrated FIGS. 28, 30, and 37.

Various Cas proteins can be used in this invention. A Cas protein, CRISPR-associated protein, or CRISPR protein, used interchangeably, refers to a protein of or derived from a CRISPR-Cas Class 1 or Class 2, including type I, type II, type III, type IV, type V, or type VI system, which has an RNA-guided DNA-binding. Non-limiting examples of suitable CRISPR/Cas proteins include Cas3, Cas4, Cas5, Cas5e (or CasD), Cash, Cas6e, Cas6f, Cas7, Cas8a1, Cas8a2, Cas8b, Cas8c, Cas9, Cas10, Cas10d, Cas13, Cas13e, Cas13f, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966. See e.g., U.S. Ser. No. 11/225,659B2, WO2021168799A1, WO2022188039A1, WO2022188797A1, WO2022068912A1, WO2022047624A1, WO2014144761 WO2014144592, WO2013176772, US20140273226, and US20140273233, the contents of which are incorporated herein by reference in their entireties.

Expression Cassettes and Expression Vectors

The disclosure also provides an expression cassette, comprising or consisting of a recombinant nucleic acid encoding an inhibitory nucleic acid or a CRISPR/Cas system described above. Where such recombinant nucleic acid may not already comprise a promoter, the expression cassette may additionally comprise a promoter. Thus, an expression cassette according to the present invention comprises, in 5′ to 3′ direction, a promoter, a coding sequence, and optionally a terminator or other elements. The expression cassette allows an easy transfer of a nucleic acid sequence of interest into an organism, preferably a cell and preferably a disease cell.

The expression cassette of the present disclosure is preferably comprised in a vector. Thus, the vector of the present disclosure allows to transform a cell with a nucleic acid sequence of interest. Correspondingly the disclosure provides a host cell comprising an expression cassette according to the present disclosure or a recombinant nucleic acid according to the present disclosure. The recombinant nucleic acid may also comprise a promoter or enhancer such as to allow for the expression of the nucleic acid sequence of interest.

Exogenous genetic material (e.g., a nucleic acid, an expression cassette, or an expression vector encoding one or more therapeutic or inhibitory RNAs) can be introduced into a target cells of interest in vivo by genetic transfer methods, such as transfection or transduction, to provide a genetically modified cell. Various expression vectors (i.e., vehicles for facilitating delivery of exogenous genetic material into a target cell) are known to one of ordinary skill in the art. As used herein, “exogenous genetic material” refers to a nucleic acid or an oligonucleotide, either natural or synthetic, that is not naturally found in the cells; or if it is naturally found in the cells, it is not transcribed or expressed at biologically significant levels by the cells. Thus, “exogenous genetic material” includes, for example, a non-naturally occurring nucleic acid that can be transcribed into an RNA.

As used herein, “transfection of cells” refers to the acquisition by a cell of new genetic material by incorporation of added nucleic acid (DNA, RNA, or a hybrid thereof) without use of a viral delivery vehicle. Thus, transfection refers to the introducing of nucleic acid into a cell using physical or chemical methods. Several transfection techniques are known to those of ordinary skill in the art including: calcium phosphate nucleic acid co-precipitation, strontium phosphate nucleic acid co-precipitation, DEAE-dextran, electroporation, cationic liposome-mediated transfection, and tungsten particle-facilitated microparticle bombardment. In contrast, “transduction of cells” refers to the process of transferring nucleic acid into a cell using a DNA or RNA virus. An RNA virus (e.g., a retrovirus) for transferring a nucleic acid into a cell is referred to herein as a transducing chimeric virus. Exogenous genetic material contained within the virus can be incorporated into the genome of the transduced cell. A cell that has been transduced with a chimeric DNA virus (e.g., an adenovirus carrying a DNA encoding a therapeutic agent), may not have the exogenous genetic material incorporated into its genome but may be capable of expressing the exogenous genetic material that is retained extrachromosomally within the cell.

Typically, the exogenous genetic material may include a heterologous gene (coding for a therapeutic RNA or protein) together with a promoter to control transcription of the new gene. The promoter characteristically has a specific nucleotide sequence necessary to initiate transcription. Optionally, the exogenous genetic material further includes additional sequences (i.e., enhancers) required to obtain the desired gene transcription activity. The exogenous genetic material may introduced into the cell genome immediately downstream from the promoter so that the promoter and coding sequence are operatively linked so as to permit transcription of the coding sequence. A retroviral expression vector may include an exogenous promoter element to control transcription of the inserted exogenous gene. Such exogenous promoters include both constitutive and inducible promoters.

Naturally-occurring constitutive promoters control the expression of essential cell functions. As a result, a gene under the control of a constitutive promoter is expressed under all conditions of cell growth. Exemplary constitutive promoters include the promoters for the following genes that encode certain constitutive or “housekeeping” functions: hypoxanthine phosphoribosyl transferase, dihydrofolate reductase, adenosine deaminase, phosphoglycerol kinase, pyruvate kinase, phosphoglycerol mutase, the actin promoter, ubiquitin, elongation factor-1 and other constitutive promoters known to those of skill in the art. In addition, many viral promoters function constitutively in eucaryotic cells. These include the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only in, or largely controlled by, the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified cell. If the gene encoding the therapeutic agent is under the control of an inducible promoter, delivery of the therapeutic agent in situ is triggered by exposing the genetically modified cell in situ to conditions for permitting transcription of the therapeutic agent, e.g., by injection of specific inducers of the inducible promoters which control transcription of the agent. For example, in situ expression by genetically modified cells of a therapeutic agent encoded by a gene under the control of the metallothionein promoter, is enhanced by contacting the genetically modified cells with a solution containing the appropriate (i.e., inducing) metal ions in situ.

Accordingly, the amount of therapeutic agent that is delivered in situ is regulated by controlling such factors as: (1) the nature of the promoter used to direct transcription of the inserted gene, (i.e., whether the promoter is constitutive or inducible, strong or weak); (2) the number of copies of the exogenous gene that are inserted into the cell; (3) the number of transduced/transfected cells that are administered (e.g., implanted) to the patient; (4) the size of the implant (e.g., graft or encapsulated expression system); (5) the number of implants; (6) the length of time the transduced/transfected cells or implants are left in place; and (7) the production rate of the therapeutic agent by the genetically modified cell. Selection and optimization of these factors for delivery of a therapeutically effective dose of a particular therapeutic agent is deemed to be within the scope of one of ordinary skill in the art without undue experimentation, taking into account the above-disclosed factors and the clinical profile of the patient.

In addition to at least one promoter and at least one heterologous nucleic acid encoding the therapeutic agent, the expression vector may include a selection gene, for example, a neomycin resistance gene or a fluorescent protein gene, for facilitating selection of cells that have been transfected or transduced with the expression vector. Alternatively, the cells are transfected with two or more expression vectors, at least one vector containing the gene(s) encoding the therapeutic agent(s), the other vector containing a selection gene. The selection of a suitable promoter, enhancer, selection gene, and/or signal sequence is deemed to be within the scope of one of ordinary skill in the art without undue experimentation.

A coding sequence of the present disclosure can be inserted into any type of target or host cell. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Carrier/Delivery of Polynucleotides

As disclosed herein, the polynucleotides or nucleic acid molecules described above can be used for treating a disorder in a subject. Accordingly, this disclosure provides systems and methods for delivery of the polynucleotides to a target cell or a subject.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

The polynucleotides or nucleic acids described herein (e.g., inhibitory nucleic acids, those encoding a CRISPR-Cas system, expression cassette, and expression vector) can be added directly, or can be complexed with cationic lipids, packaged within liposomes, or as a recombinant plasmid or viral vectors, or otherwise delivered to target cells or tissues. Methods for the delivery of nucleic acid molecules are known in the art. See, e.g., U.S. Pat. No. 6,395,713, WO 94/02595, Akhtar et al., 1992, Trends Cell Bio., 2, 139; Delivery Strategies for Antisense Oligonucleotide Therapeutics, ed. Akhtar, 1995, Maurer et al., 1999, Mol. Membr. Biol., 16, 129-140; Hofland and Huang, 1999, Handb. Exp. Pharmacol., 137, 165-192; and Lee et al., 2000, ACS Symp. Ser., 752, 184-192. These protocols can be utilized for the delivery of virtually any nucleic acid molecule. Nucleic acid molecules can be administered to cells by a variety of methods known to those of skill in the art, including, but not restricted to, encapsulation in liposomes, by iontophoresis, or by incorporation into other vehicles, such as biodegradable polymers, hydrogels, cyclodextrins (see for example Gonzalez et al., 1999, Bioconjugate Chem., 10, 1068-1074; WO 03/47518 and WO 03/46185), poly(lactic-co-glycolic)acid (PLGA) and PLCA microspheres (see for example U.S. Pat. No. 6,447,796 and US 2002130430), biodegradable nanocapsules, and bioadhesive microspheres, or by proteinaceous vectors (see, e.g., WO 00/53722).

In one aspect, the present application provides carrier systems containing the nucleic acid molecules described herein. In some embodiments, the carrier system is a lipid-based carrier system, cationic lipid, or liposome nucleic acid complexes, a liposome, a micelle, a virosome, a lipid nanoparticle or a mixture thereof. In other embodiments, the carrier system is a polymer-based carrier system such as a cationic polymer-nucleic acid complex. In additional embodiments, the carrier system is a cyclodextrin-based carrier system such as a cyclodextrin polymer-nucleic acid complex. In further embodiments, the carrier system is a protein-based carrier system such as a cationic peptide-nucleic acid complex. Preferably, the carrier system in a lipid nanoparticle formulation. Lipid nanoparticle (“LNP”) formulations described herein can be applied to any nucleic acid molecules (e.g., an RNA molecule) or combination of nucleic acid molecules described herein.

In certain embodiment, the nucleic acid molecules described herein are formulated as a lipid nanoparticle composition such as is described in U.S. Pat. Nos. 7,514,099 and 7,404,969. In some embodiments, this application features a composition comprising a nucleic acid molecule formulated as any of formulation as described in US 20120029054, such as LNP-051; LNP-053; LNP-054; LNP-069; LNP-073; LNP-077; LNP-080; LNP-082; LNP-083; LNP-060; LNP-061; LNP-086; LNP-097; LNP-098; LNP-099; LNP-100; LNP-101; LNP-102; LNP-103; or LNP-104.

In other embodiments, this disclosure features conjugates and/or complexes of nucleic acid molecules described herein. Such conjugates and/or complexes can be used to facilitate delivery of nucleic acid molecules into a biological system, such as a cell. The conjugates and complexes provided by hereon can impart therapeutic activity by transferring therapeutic compounds across cellular membranes, altering the pharmacokinetics, and/or modulating the localization of nucleic acid molecules of the invention. Non-limiting, examples of such conjugates are described in e.g., U.S. Pat. Nos. 7,833,992; 6,528,631; 6,335,434; 6, 235,886; 6,153,737; 5,214,136; 5,138,045.

In various embodiments, polyethylene glycol (PEG) can be covalently attached to nucleic acid molecules described herein. The attached PEG can be any molecular weight, preferably from about 100 to about 50,000 daltons (Da). Accordingly, the disclosure features compositions or formulations comprising surface-modified liposomes containing poly (ethylene glycol) lipids (PEG-modified, or long-circulating liposomes or stealth liposomes) and nucleic acid molecules described herein. See, e.g., WO 96/10391, WO 96/10390, and WO 96/10392).

In some embodiments, the nucleic acid molecules can also be formulated or complexed with polyethyleneimine and derivatives thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives. In one embodiment, the nucleic acid molecules can be formulated in the manner described in U.S. 20030077829.

In other embodiments, nucleic acid molecules described herein can be complexed with membrane disruptive agents such as those described in U.S. 20010007666. In still other embodiments, the membrane disruptive agent or agents and the molecule can be complexed with a cationic lipid or helper lipid molecule, such as those lipids described in U.S. Pat. No. 6,235,310.

In certain embodiments, nucleic acid molecules described herein can be complexed with delivery systems as described in U.S. Patent Application Publication Nos. 2003077829; 20050287551; 20050164220; 20050191627; 20050118594; 20050153919; 20050085486; and 20030158133; and IWO 00/03683 and WO 02/087541.

In some embodiments, a liposomal formulation described herein can comprise a nucleic acid molecule described herein (e.g., an inhibitory nucleic acid) formulated or complexed with compounds and compositions described in U.S. Pat. Nos. 6,858,224; 6,534,484; 6,287,591; 6,835,395; 6,586,410; 6,858,225; 6,815,432; 6,586,001; 6,120,798; 6,977,223; 6,998,115; 5,981,501; 5,976,567; 5,705,385; and U.S. Patent Application Publication Nos. 2006/0019912; 2006/0019258; 2006/0008909; 2005/0255153; 2005/0079212; 2005/0008689; 2003/0077829, 2005/0064595, 2005/0175682, 2005/0118253; 2004/0071654; 2005/0244504; 2005/0265961 and 2003/0077829.

As disclosed herein, the nucleic acid molecules described above can be used for treating a disorder in a subject. Vectors (such as recombinant plasmids and viral vectors) as discussed above can be used to deliver a therapeutical agent, such as an inhibitory nucleic acid or a CRISPR-Cas system described herein. Delivery of the vectors can be systemic, such as by intravenous or intra-muscular administration, by administration to target cells ex-planted from a subject followed by reintroduction into the subject, or by any other means that would allow for introduction into the desired target cell. Such recombinant vectors can also be administered directly or in conjunction with a suitable delivery reagents, including, for example, the Minis Transit LT1 lipophilic reagent; lipofectin; lipofectamine; cellfectin; polycations (e.g., polylysine) or liposomes lipid-based carrier system, cationic lipid, or liposome nucleic acid complexes, a micelle, a virosome, a lipid nanoparticle.

Viral Vectors

In some embodiments, a polynucleotide encoding the RNA molecule can be inserted into, or encoded by, vectors such as plasmids or viral vectors. Preferably, the polynucleotide is inserted into, or encoded by, viral vectors. Viral vectors may be Herpesvirus (HSV) vectors, retroviral vectors, adenoviral vectors, AAV vectors, lentiviral vectors, and the like. In some specific embodiments, the viral vectors are AAV vectors. In some embodiments, the RNA may be encoded by a retroviral vector (See, e.g., U.S. Pat. Nos. 5,399,346; 5,124,263; 4,650,764 and 4,980,289; the content of each of which is incorporated herein by reference in their entirety).

Lentiviral Vectors

Lentiviruses, such as HIV, are “slow viruses.” Vectors derived from lentiviruses can be expressed long-term in the host cells after a few administrations to the patients, e.g., via ex vivo transduced stem cells or progenitor cells. For most diseases and disorders, including genetic diseases, cancer, and neurological disease, long-term expression is crucial to successful treatment. Regarding safety with lentiviral vectors, a number of strategies for eliminating the ability of lentiviral vectors to replicate have now been known in the art. See e.g., US 20210401868 and 20210403517, each of which is incorporated herein by reference in its entirety. For example, the deletion of promoter and enhancer elements from the U3 region of the long terminal repeat (LTR) are thought to have no LTR-directed transcription. The resulting vectors are called “self-inactivating” (SIN).

Lentiviral vectors are particularly suitable to achieving long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as CNS cells. They also have the added advantage of low immunogenicity. In general, a suitable vector contains an origin of replication functional in at least one organism, a promoter sequence, convenient restriction endonuclease sites, and one or more selectable markers, (e.g., WO01/96584 and WO01/29058; and U.S. Pat. No. 6,326,193). Several vector promoter sequences are available for expression of the transgenes. One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is EF1a. However, other constitutive promoter sequences can also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Inducible promoters include, but are not limited to a metallothionein promoter, a glucocorticoid promoter, a progesterone promoter, and a tetracycline promoter.

The present disclosure provides a recombinant lentivirus capable of infecting dividing and non-dividing cells, such oligodendrocytes, astrocytes, or glial progenitor cells. The virus is useful for the in vivo and ex vivo transfer and expression of nucleic acid sequences. Lentiviral vectors of the present disclosure may be lentiviral transfer plasmids or infectious lentiviral particles. Construction of lentiviral vectors, helper constructs, envelope constructs, etc., for use in lentiviral transfer systems has been described in, e.g., US 20210401868 and 20210403517, each of which is incorporated herein by reference in its entirety.

Adenoviruses

Adenoviruses are eukaryotic DNA viruses that can be modified to efficiently deliver a nucleic acid to a variety of cell types in vivo, and have been used extensively in gene therapy protocols, including for targeting genes to neural cells and glial cells. Various replication defective adenovirus and minimum adenovirus vectors have been described for nucleic acid therapeutics (See, e.g., PCT Patent Publication Nos. WO199426914, WO 199502697, WO199428152, WO199412649, WO199502697 and WO199622378; the content of each of which is incorporated by reference in their entirety). Such adenoviral vectors may also be used to deliver therapeutic molecules of the present disclosure to cells.

Adeno Associated Virus

The adeno-associated virus is a widely used gene therapy vector due to its clinical safety record, non-pathogenic nature, ability to infect non-dividing cells (like neurons), and ability to provide long-term gene expression after a single administration. Currently, many human and non-human primate AAV serotypes have been identified. AAV vectors have demonstrated safety in hundreds of clinical trials worldwide, and clinical efficacy has been shown in trials of hemophilia B, spinal muscular atrophy, alpha 1 antitrypson, and Leber congenital amaurosis.

Because of their safety, nonpathogenic nature, and ability to infect neurons, AAVs such as AAV1, AAV2, AAV4, AAV5, AAV6, AAV8, and AAV9 are commonly used gene therapy vectors for CNS applications. However, after direct CNS infusion, these serotypes exhibit a dominant neuronal tropism and expression in oligodendrocytes is low, especially when gene expression is driven by a constitutive promoter, which restricts their potential for use in treating white matter diseases. AAV1/2, AAV2, and AAV8 have been shown transduce oligodendrocytes. Reliance on cell-specific promoters for expression specificity allows for the possibility of nonselective cellular uptake and leaky transgene expression through cryptic promoter activity in non-oligodendrocyte lineage cells.

The approach described herein to alleviate these issues includes using AAV serotypes with high tropism for oligodendrocytes or astrocytes or glial progenitor cells. Recently, using DNA shuffling and directed evolution, a chimeric AAV capsid with strong selectivity for oligodendrocytes, AAV/Olig001, has been described (Powell et al., 2016, Gene Ther 23:807-814). Subsequently, AAV/Olig001 was shown to transduce neonatal oligodendrocytes in a mouse model of Canavan disease (Francis et al., 2021. Mol Ther Methods Clin Dev 20:520-534). Other approaches such as random mutagenesis and peptide library insertion can be used to generate capsid libraries that can be screened for tropism and selectivity for oligodendrocytes or astrocytes or glial progenitor cells.

As discussed above, the terms “adeno-associated virus” and/or “AAV” refer to parvoviruses with a linear single-stranded DNA genome and variants thereof. The term covers all subtypes and both naturally occurring and recombinant forms, except where required otherwise. Parvoviruses, including AAV, are useful as gene therapy vectors as they can penetrate a cell and introduce a nucleic acid (e.g., transgene) into the nucleus. In some embodiments, the introduced nucleic acid (e.g., rAAV vector genome) forms circular concatemers that persist as episomes in the nucleus of transduced cells. In some embodiments, a transgene is inserted in specific sites in the host cell genome. Site-specific integration, as opposed to random integration, is believed to likely result in a predictable long-term expression profile. The insertion site of AAV into the human genome is referred to as AAVS1. Once introduced into a cell, RNAs or polypeptides encoded by the nucleic acid can be expressed by the cell. Because AAV is not associated with any pathogenic disease in humans, a nucleic acid delivered by AAV can be used to express a therapeutic RNA or polypeptide for the treatment of a disease, disorder and/or condition in a human subject.

Multiple serotypes of AAV exist in nature with at least fifteen wild type serotypes having been identified from humans thus far (i.e., AAV1-AAV15). Naturally occurring and variant serotypes are distinguished by having a protein capsid that is serologically distinct from other AAV serotypes. Examples include AAV1, AAV2, AAV, AAV3 (including AAV3A and AAV3B), AAV4, AAV5, AAV6, AAV7, AAV8, AAV9, AAV10, AAV12, AAVrh10, AAVrh74 (see WO 2016/210170), avian AAV, bovine AAV, canine AAV, equine AAV, primate AAV, non-primate AAV, and ovine AAV, and recombinantly produced variants (e.g., capsid variants with insertions, deletions and substitutions, etc.), such as variants referred to as AAV2i8, NP4, NP22, NP66, DJ, DJ/8, DJ/9, LK3, RHM4-1, among many others. “Primate AAV” refers to AAV that infect primates, “non-primate AAV” refers to AAV that infect non-primate mammals, “bovine AAV” refers to AAV that infect bovine mammals, and so on.

Serotype distinctiveness is determined on the basis of the lack of cross-reactivity between antibodies to one AAV as compared to another AAV. Such cross-reactivity differences are usually due to differences in capsid protein sequences and antigenic determinants (e.g., due to VP1, VP2, and/or VP3 sequence differences of AAV serotypes). However, some naturally occurring AAV or man-made AAV mutants (e.g., recombinant AAV) may not exhibit serological difference with any of the currently known serotypes. These viruses may then be considered a subgroup of the corresponding type, or more simply a variant AAV. Thus, as used herein, the term “serotype” refers to both serologically distinct viruses, as well as viruses that are not serologically distinct but that may be within a subgroup or a variant of a given serotype.

A comprehensive list and alignment of amino acid sequences of capsids of known AAV serotypes is provided by Marsic et al. (2014) Molecular Therapy 22(11):1900-1909. Genomic sequences of various serotypes of AAV, as well as sequences of the native ITRs, rep proteins, and capsid subunits are known in the art. Such sequences may be found in the literature or in public databases such as GenBank. See, e.g., GenBank Accession Numbers NC_002077 (AAV1), AF063497 (AAV1), NC_001401 (AAV2), AF043303 (AAV2), NC_001729 (AAV3), NC_001863 (AAV3B), NC_001829 (AAV4), U89790 (AAV4), NC_006152 (AAV5), NC_001862 (AAV6), AF513851 (AAV7), AF513852 (AAV8), and NC_006261 (AAV8); the disclosures of which are incorporated by reference herein. See also, e.g., Srivistava et al. (1983) J. Virology 45:555; Chiorini et al. (1998) J. Virology 71:6823; Chiorini et al. (1999) J. Virology 73: 1309; Bantel-Schaal et al. (1999) J. Virology 73:939; Xiao et al. (1999) J. Virology 73:3994; Muramatsu et al. (1996) Virology 221:208; Shade et al. (1986) J. Virol. 58:921; Gao et al. (2002) Proc. Nat. Acad. Sci. USA 99: 11854; Moris et al. (2004) Virology 33:375-383; international patent publications WO 00/28061, WO 99/61601, WO 98/11244; WO 2013/063379; WO 2014/194132; WO 2015/121501, and U.S. Pat. Nos. 6,156,303 and 7,906,111.

As discussed herein, a “recombinant adeno-associated virus” or “rAAV” is distinguished from a wild-type AAV by replacement of all or part of the endogenous viral genome with a non-native sequence. Incorporation of a non-native sequence within the virus defines the viral vector as a “recombinant” vector, and hence a “rAAV vector.” An rAAV vector can include a heterologous polynucleotide encoding a desired RNA or protein or polypeptide (e.g., an RNA molecule disclosed herein). A recombinant vector sequence may be encapsidated or packaged into an AAV capsid and referred to as an “rAAV vector,” an “rAAV vector particle,” “rAAV viral particle” or simply a “rAAV.”

The present disclosure provides for an rAAV vector comprising a polynucleotide sequence not of AAV origin (e.g., a polynucleotide heterologous to AAV). The heterologous polynucleotide may be flanked by at least one, and sometimes by two, AAV terminal repeat sequences (e.g., inverted terminal repeats). The heterologous polynucleotide flanked by ITRs, also referred to herein as a “vector genome,” typically encodes an RNA or a polypeptide of interest, or a gene of interest, such as a target for therapeutic treatment. Delivery or administration of an rAAV vector to a subject (e.g. a patient) provides encoded RNAs/proteins/peptides to the subject. Thus, an rAAV vector can be used to transfer/deliver a heterologous polynucleotide for expression for, e.g., treating a variety of diseases, disorders and conditions.

rAAV vector genomes generally retain 145 base ITRs in cis to the heterologous nucleic acid sequence that replaced the viral rep and cap genes. Such ITRs are useful to produce a recombinant AAV vector; however, modified AAV ITRs and non-AAV terminal repeats including partially or completely synthetic sequences can also serve this purpose. ITRs form hairpin structures and function to, for example, serve as primers for host-cell-mediated synthesis of the complementary DNA strand after infection. ITRs also play a role in viral packaging, integration, etc. ITRs are the only AAV viral elements which are required in cis for AAV genome replication and packaging into rAAV vectors. An rAAV vector genome optionally comprises two ITRs which are generally at the 5′ and 3′ ends of the vector genome comprising a heterologous sequence (e.g., a transgene encoding a gene of interest, or a nucleic acid sequence of interest including, but not limited to, an antisense, and siRNA, a CRISPR molecule, among many others). A 5′ and a 3′ ITR may both comprise the same sequence, or each may comprise a different sequence. An AAV ITR may be from any AAV including by not limited to serotypes 1, 2, 3, 4, 5, 6, 7, 8, 9, 10 or 11 or any other AAV.

An rAAV vector of the disclosure may comprise an ITR from an AAV serotype (e.g., wild-type AAV2, a fragment or variant thereof) that differs from the serotype of the capsid (e.g., AAV8, Olig001). Such an rAAV vector comprising at least one ITR from one serotype, but comprising a capsid from a different serotype, may be referred to as a hybrid viral vector (see U.S. Pat. No. 7,172,893). An AAV ITR may include the entire wild type ITR sequence, or be a variant, fragment, or modification thereof, but will retain functionality.

In some embodiments, an rAAV vector genome is linear, single-stranded and flanked by AAV ITRs. Prior to transcription and translation of the heterologous gene, a single stranded DNA genome of approximately 4700 nucleotides must be converted to a double-stranded form by DNA polymerases (e.g., DNA polymerases within the transduced cell) using the free 3′-OH of one of the self-priming ITRs to initiate second-strand synthesis. In some embodiments, full length-single stranded vector genomes (i.e., sense and anti-sense) anneal to generate a full length-double stranded vector genome. This may occur when multiple rAAV vectors carrying genomes of opposite polarity (i.e., sense or anti-sense) simultaneously transduce the same cell. Regardless of how they are produced, once double-stranded vector genomes are formed, the cell can transcribe and translate the double-stranded DNA and express the heterologous gene.

The efficiency of transgene expression from an rAAV vector can be hindered by the need to convert a single stranded rAAV genome (ssAAV) into double-stranded DNA prior to expression. This step can be circumvented by using a self-complementary AAV genome (scAAV) that can package an inverted repeat genome that can fold into double-stranded DNA without the need for DNA synthesis or base-pairing between multiple vector genomes. See, e.g., U.S. Pat. No. 8,784,799; McCarty, (2008) Molec. Therapy 16(10):1648-1656; and McCarty et al., (2001) Gene Therapy 8:1248-1254; McCarty et al., (2003) Gene Therapy 10:2112-2118.

A viral capsid of an rAAV vector may be from a wild type AAV or a variant AAV such as AAV1, AAV2, AAV3, AAV3A, AAV3B, AAV4, AAV5, AAV6, AAV7, AAV8, AAV9, AAV10, AAVrh10, AAVrh74 (see WO2016/210170), AAV12, AAV2i8, AAV1.1, AAV2.5, AAV6.1, AAV6.3.1, AAV9.45, RHM4-1 (WO 2015/013313), RHM15-1, RHM15-2, RHM15-3/RHM15-5, RHM15-4, RHM15-6, AAV hu.26, AAV1.1, AAV2.5, AAV6.1, AAV6.3.1, AAV9,45, AAV2i8, AAV29G, AAV2,8G9, AVV-LK03, AAV2-TT, AAV2-TT-S312N, AAV3B-S312N, AAV avian AAV, bovine AAV, canine AAV, equine AAV, primate AAV, non-primate AAV, snake AAV, goat AAV, shrimp AAV, ovine AAV and variants thereof (see, e.g., Fields et al., VIROLOGY, volume 2, chapter 69 (4th ed., Lippincott-Raven Publishers). Capsids may be derived from a number of AAV serotypes disclosed in U.S. Pat. No. 7,906,111; Gao et al. (2004) J. Virol. 78:6381; Morris et al. (2004) Virol. 33:375; WO 2013/063379; WO 2014/194132; and include true type AAV (AAV-TT) variants disclosed in WO 2015/121501, and RHM4-1, RHM15-1 through RHM15-6, and variants thereof, disclosed in WO 2015/013313. A full complement of AAV cap proteins includes VP1, VP2, and VP3. The ORF comprising nucleotide sequences encoding AAV VP capsid proteins may comprise less than a full complement AAV Cap proteins or the full complement of AAV cap proteins may be provided.

In some embodiments, an rAAV vector comprising a capsid protein encoded by a nucleotide sequence derived from more than one AAV serotype (e.g., wild type AAV serotypes, variant AAV serotypes) is referred to as a “chimeric vector” or “chimeric capsid” (See U.S. Pat. No. 6,491,907, the entire disclosure of which is incorporated herein by reference). In some embodiments, a chimeric capsid protein is encoded by a nucleic acid sequence derived from 2, 3, 4, 5, 6, 7, 8, 9, 10 or more AAV serotypes. In some embodiments, a recombinant AAV vector includes a capsid sequence derived from e.g., AAV1, AAV2, AAV3, AAV3A, AAV3B, AAV4, AAV5, AAV6, AAV7, AAV8, AAV9, AAV10, AAV11, AAVrh74, AAVrh10, AAV2i8, or variant thereof, resulting in a chimeric capsid protein comprising a combination of amino acids from any of the foregoing AAV serotypes (see, Rabinowitz et al. (2002) J. Virology 76(2):791-801). Alternatively, a chimeric capsid can comprise a mixture of a VP1 from one serotype, a VP2 from a different serotype, a VP3 from yet a different serotype, and a combination thereof. For example a chimeric virus capsid may include an AAV1 cap protein or subunit and at least one AAV2 cap protein or subunit. A chimeric capsid can, for example include an AAV capsid with one or more B19 cap subunits, e.g., an AAV cap protein or subunit can be replaced by a B19 cap protein or subunit. For example, in one embodiment, a VP3 subunit of an AAV capsid can be replaced by a VP2 subunit of B19. In some embodiments, a chimeric capsid is an Olig001 capsid as described in WO2021221995 and WO2014052789, which are incorporated herein by reference.

In some embodiments, chimeric vectors have been engineered to exhibit altered tropism or tropism for a particular tissue or cell type. The term “tropism” refers to preferential entry of the virus into certain cell (e.g., oligodendrocytes) or tissue types and/or preferential interaction with the cell surface that facilitates entry into certain cell or tissue types. AAV tropism is generally determined by the specific interaction between distinct viral capsid proteins and their cognate cellular receptors (Lykken et al. (2018) J. Neurodev. Disord. 10:16). Preferably, once a virus or viral vector has entered a cell, sequences (e.g., heterologous sequences such as a transgene) carried by the vector genome (e.g., an rAAV vector genome) are expressed.

A “tropism profile” refers to a pattern of transduction of one or more target cells in various tissues and/or organs. For example, a chimeric AAV capsid may have a tropism profile characterized by efficient transduction of oligodendrocytes or astrocytes or oligodendrocyte progenitor cells with only low transduction of neurons and other CNS cells. See WO2014/052789, incorporated herein by reference. Such a chimeric capsid may be considered specific for oligodendrocytes or astrocytes or glial progenitor cells exhibiting tropism for oligodendrocytes or astrocytes or glial progenitor cells, and referred to herein as “glialtropism,” if when administered directly into the CNS, preferentially transduces oligodendrocytes or astrocytes or oligodendrocyte progenitor cells over neurons and other CNS cell types. In some embodiments, at least about 80% of cells that are transduced by a capsid specific for oligodendrocytes or oligodendrocyte progenitor cells are oligodendrocytes or oligodendrocyte progenitor cells, e.g., at least about 85%, 90%, 95%, 96%, 97%, 98% 99% or more of the transduced cells are oligodendrocytes or oligodendrocyte progenitor cells.

Cell Replacement Therapy

One aspect of the present application relates to a method of alleviating adverse effects of age-related oligodendrocyte loss, astrocyte loss, or white matter loss in the CNS (e.g., brain) of an adult subject. This method includes identifying a subject, e.g., an adult subject, undergoing adverse effects of age-related oligodendrocyte loss, astrocyte loss, or white matter loss in the CNS (e.g., brain) and providing a population of isolated glial progenitor cells. The population of isolated glial progenitor cells is then introduced into CNS (such as the brain and/or brain stem) of the selected subject to at least partially replace cells in the subject's brain in the location undergoing the adverse effects of age-related white matter loss.

As used herein, the term “glial cells” refers to a population of non-neuronal cells that provide support and nutrition, maintain homeostasis, either form myelin or promote myelination, and participate in signal transmission in the nervous system. “Glial cells” as used herein encompasses fully differentiated cells of the glial lineage, such as oligodendrocytes or astrocytes, and as well as glial progenitor cells. Glial progenitor cells are cells having the potential to differentiate into cells of the glial lineage such as oligodendrocytes and astrocytes.

The glial progenitor cells described herein may be derived from any suitable source of pluripotent stem cells, such as, for example and without limitation, human induced pluripotent stem cells (iPSCs) and embryonic stem cells, as described in more detail below. In one example, glial progenitor cells can be cells rejuvenated from glial progenitor cells or progenies thereof as described herein.

In some embodiments, to treat a subject in need thereof, glial progenitor cells or rejuvenated cells are young glial or glial progenitor cells, or are younger than the counterparts in the subject to be treated.

As used herein the term “young” glial or glial progenitor cells refers to cells that are induced to start differentiation into glial progenitor cell in an in vitro setting (about 105 days from cell isolation from fetal donor tissue). In some embodiments, the term “young glial cells” refers to differentiated glial progenitor cells that are ready for transplantation into an animal (about 160 days from cell isolation from fetal donor tissue). In some embodiments, the term “young glial cells” refers to glial progenitor cells or their progeny that are within 1-weeks of transplantation. The term “older glial cells” is used in relative to the term “young glial cells”. Compared with older glial cells, young glial cells may have one or more of the following characteristics: (i) growing or proliferating or dividing faster, (ii) having lower levels than old of senescence-associated transcripts encoding CDKN1A (p21Cip1) and CDKN2/p16(INK4) and p14(ARF), and (iii) longer telomeres or higher telomerase activity or both.

In some embodiments, older glial cells are glial cells that are derived from glial progenitor cells that have been transplanted into a host for 5, 10, 20, 30 or 40 weeks. In some embodiments, the older glial cells are glial cells that have been cultured for an additional 5, 10, 20, 30 or 40 weeks from differentiated glial progenitor cells (e.g., about 160 days from the initial tissue harvest). In some embodiments, the older glial cells are glial cells that have been cultured for an additional 5, 10, 20, 30 or 40 weeks from the introduction of differentiation (e.g., about 105 days from the initial tissue harvest).

iPSCs are pluripotent cells that are derived from non-pluripotent cells, such as somatic cells. For example, and without limitation, iPSCs can be derived from tissue, peripheral blood, umbilical cord blood, and bone marrow (see e.g., Cai et al., J. Biol. Chem. 285(15):112227-11234 (2110); Giorgetti et al., Nat. Protocol. 5(4):811-820 (2010); Streckfuss-Bomeke et al., Eur. Heart J. doi:10.1093/eurheartj/ehs203 (Jul. 12, 2012); Hu et al., Blood doi:10.1182/blood-2010-07-298331 (Feb. 4, 2011); Sommer et al., J. Vis. Exp. 68:e4327 doi:10.3791/4327 (2012), which are hereby incorporated by reference in their entirety). The somatic cells can be reprogrammed to an embryonic stem cell-like state using genetic manipulation. Exemplary somatic cells suitable for the formation of iPSCs include fibroblasts (see e.g., Streckfuss-Bomeke et al., Eur. Heart J. doi:10.1093/eurheartj/ehs203 (2012), which is hereby incorporated by reference in its entirety), such as dermal fibroblasts obtained by a skin sample or biopsy, synoviocytes from synovial tissue, keratinocytes, mature B cells, mature T cells, pancreatic β cells, melanocytes, hepatocytes, foreskin cells, cheek cells, or lung fibroblasts.

Methods of producing induced pluripotent stem cells are known in the art and typically involve expressing a combination of reprogramming factors in a somatic cell. Suitable reprogramming factors that promote and induce iPSC generation include one or more of Oct4, Klf4, Sox2, c-Myc, Nanog, C/EBPα, Esrrb, Lin28, and Nr5a2. In certain embodiments, at least two reprogramming factors are expressed in a somatic cell to successfully reprogram the somatic cell. In other embodiments, at least three reprogramming factors are expressed in a somatic cell to successfully reprogram the somatic cell.

iPSCs may be derived by methods known in the art, including the use integrating viral vectors (e.g., lentiviral vectors, inducible lentiviral vectors, and retroviral vectors), excisable vectors (e.g., transposon and floxed lentiviral vectors), and non-integrating vectors (e.g., adenoviral and plasmid vectors) to deliver the genes that promote cell reprogramming (see e.g., Takahashi and Yamanaka, Cell 126:663-676 (2006); Okita. et al., Nature 448:313-317 (2007); Nakagawa et al., Nat. Biotechnol. 26:101-106 (2007); Takahashi et al., Cell 131:1-12 (2007); Meissner et al. Nat. Biotech. 25:1177-1181 (2007); Yu et al. Science 318:1917-1920 (2007); Park et al. Nature 451:141-146 (2008); and U.S. Patent Application Publication No. 2008/0233610, which are hereby incorporated by reference in their entirety). Other methods for generating IPS cells include those disclosed in WO2007/069666, WO2009/006930, WO2009/006997, WO2009/007852, WO2008/118820, U.S. Patent Application Publication No. 2011/0200568 to Ikeda et al., U.S. Patent Application Publication No 2010/0156778 to Egusa et al., U.S. Patent Application Publication No 2012/0276070 to Musick, and U.S. Patent Application Publication No 2012/0276636 to Nakagawa, Shi et al., Cell Stem Cell 3(5):568-574 (2008), Kim et al., Nature 454:646-650 (2008), Kim et al., Cell 136(3):411-419 (2009), Huangfu et al., Nat. Biotechnol. 26:1269-1275 (2008), Zhao et al., Cell Stem Cell 3:475-479 (2008), Feng et al., Nat. Cell Biol. 11:197-203 (2009), and Hanna et al., Cell 133(2):250-264 (2008) which are hereby incorporated by reference in their entirety.

The methods of iPSC generation described above can be modified to include small molecules that enhance reprogramming efficiency or even substitute for a reprogramming factor. These small molecules include, without limitation, epigenetic modulators such as, the DNA methyltransferase inhibitor 5′-azacytidine, the histone deacetylase inhibitor VPA, and the G9a histone methyltransferase inhibitor BIX-01294 together with BayK8644, an L-type calcium channel agonist. Other small molecule reprogramming factors include those that target signal transduction pathways, such as TGF-β inhibitors and kinase inhibitors (e.g., kenpaullone) (see review by Sommer and Mostoslaysky, Stem Cell Res. Ther. 1:26 doi:10.1186/scrt26 (Aug. 10, 2010), which is hereby incorporated by reference in its entirety).

Methods of obtaining highly enriched preparations of glial progenitor cells from the iPSCs that are suitable for making the non-human mammal models described herein are disclosed in WO2014/124087 to Goldman and Wang, and Wang et al., Cell Stem Cell 12(2):252-264 (2013), which are hereby incorporated by reference in their entirety.

In another embodiment of the present application, the glial progenitor cells are derived from embryonic stem cells. Embryonic stem cells are derived from totipotent cells of the early mammalian embryo and are capable of unlimited, undifferentiated proliferation in vitro. As used herein, the term “embryonic stem cells” refer to a cells isolated from an embryo, placenta, or umbilical cord, or an immortalized version of such a cells, i.e., an embryonic stem cell line. Suitable embryonic stem cell lines include, without limitation, lines WA-01 (H1), WA-07, WA-09 (H9), WA-13, and WA-14 (H14) (Thomson et al., Science 282 (5391): 1145-47 (1998) and U.S. Pat. No. 7,029,913 to Thomson et al., which are hereby incorporated by reference in their entirety). Other suitable embryonic stem cell lines includes the HAD-C100 cell line (Tannenbaum et al., PLoS One 7(6):e35325 (2012), which is hereby incorporated by reference in its entirety, the WIBR4, WIBR5, WIBR6 cel lines (Lengner et al., Cell 141(5):872-83 (2010), which is hereby incorporated by reference in its entirety), and the human embryonic stem cell lines (HUES) lines 1-17 (Cowan et al., N. Engl. J. Med. 350:1353-56 (2004), which is hereby incorporated by reference in its entirety).

Human embryonic stem cells provide a virtually unlimited source of clonal/genetically modified cells potentially useful for tissue replacement therapies. Methods of obtaining highly enriched preparations of glial progenitor cells from embryonic cells that are suitable for making the non-human mammal model of the present disclosure are described herein as disclosed in Wang et al., Cell Stem Cell 12:252-264 (2013), which is hereby incorporated by reference in its entirety.

Briefly, glial progenitor cells are derived from a pluripotent population of cells, i.e., iPSCs or embryonic stem cells, using a protocol that directs the pluripotent cells through serial stages of neural and glial progenitor cell differentiation. Each stage of lineage restriction is characterized and identified by the expression of certain cell proteins. Stage 1 of this process involves culturing the pluripotent cell population under conditions effective to induce embryoid body formation. As described herein, the pluripotent cell population may be maintained in co-culture with other cells, such as embryonic fibroblasts, in an embryonic stem cell (ESC) media (e.g., DMEM/F12 containing a suitable serum replacement and bFGF). The pluripotent cells are passaged before reaching 100% confluence, e.g., 80% confluence, when colonies are approximately 250-300 μm in diameter. The pluripotential state of the cells is readily assessed using markers to SSEA4, TRA-1-60, OCT-4, NANOG, and/or SOX2.

To generate embryoid bodies (EBs) (Stage 2), which are complex three-dimensional cell aggregates of pluripotent stem cells, pluripotent cell cultures are dissociated once they achieved ˜80% confluence with colony diameters at or around 250-300 μm. The EBs are initially cultured in suspension in ESC media without bFGF, and then switched to neural induction medium supplemented with bFGF and heparin. To induce neuroepithelial differentiation (Stage 3) EBs are plated and cultured in neural induction medium supplemented with bFGF, heparin, laminin, then switched to neural induction media supplemented with retinoic acid. Neuroepithelial differentiation is assessed by the co-expression of PAX6 and SOX1, which characterize central neural stem and progenitor cells.

To induce pre-oligodendrocyte progenitor cell (“pre-OPCs”) differentiation, neuroepithelial cell colonies can be cultured in the presence of additional factors including retinoic acid, B27 supplement, and a sonic hedgehog (shh) agonist (e.g., purmophamine). The appearance of pre-OPC colonies is assessed by the presence of OLIG2 and/or NKX2.2 expression. While both OLIG2 and NKX2.2 are expressed by central oligodendrocyte progenitor cells, NKX2.2 is a more specific indicator of oligodendroglial differentiation. Accordingly, an early pre-oligodendrocyte progenitor cell stage is marked by OLIG⁺/NKX2.2⁻ cell colonies. OLIG⁺/NKX2.2⁻ early pre-OPCs are differentiated into later-stage OLIG⁺/NKX2.2⁺ pre-OPCs by replacing retinoic acid with bFGF. At the end of Stage 5, a significant percentage of the cells are pre-OPCs as indicated by OLIG2⁺/NKX2.2⁺ expression profile.

Pre-OPCs can be further differentiated into bipotential glial progenitor cells by culture in glial induction media supplemented with growth factors such as triiodothyronine (T3), neurotrophin 3 (NT3), insulin growth factor (IGF-1), and platelet-derived growth factor-AA (PDGF-AA) (Stage 6). These culture conditions can be extended for 3-4 months or longer to maximize the production of myelinogenic glial progenitor cells when desired. Cell preparations suitable for transplantation into an appropriate subject are identified as containing PDGFRα⁺ glial progenitor cells.

The population of glial progenitor cells used in carrying out the method of the present application may comprise at least about 80% glial progenitor cells, including, for example, about 80%, 85%, 90%, 95%, 96%, 97%, 98%, 99%, 100% glial cells. The selected preparation of glial progenitor cells can be relatively devoid (e.g., containing less than 20, 15, 10, 9, 8, 7, 6, 5, 4, 3, 2, or 1%) of other cells types such as neurons and neuronal progenitor cells. Optionally, the cell population can be a substantially pure populations of glial progenitor cells.

The subject being treated in accordance with the method of the present application can be an adult afflicted with age-related white matter/oligodendrocyte/astrocyte loss in the brain. This method alleviates the adverse effects of this condition which can arise as part of the normal aging process.

As used herein, “treating” or “treatment” refers to any indication of success in amelioration of an injury, pathology, or condition, including any objective or subjective parameter such as abatement; remission; diminishing of symptoms or making the injury, pathology, or condition more tolerable to the patient; slowing the rate of degeneration or decline; making the final point of degeneration less debilitating; or improving a subject's physical or mental well-being. The treatment or amelioration of symptoms can be based on objective or subjective parameters; including the results of a physical examination, neurological examination, and/or psychiatric evaluation.

“Treating” may include the administration of glial progenitor cells or/and other agent(s) to prevent or delay, to alleviate, or to arrest or inhibit development of the symptoms or conditions associated with the disease, condition or disorder. “Therapeutic effect” refers to the reduction, elimination, or prevention of the disease, symptoms of the disease, or side effects of a disease, condition or disorder in the subject. Treatment may be prophylactic (to prevent or delay the onset or worsening of the disease, condition or disorder, or to prevent the manifestation of clinical or subclinical symptoms thereof) or therapeutic suppression or alleviation of symptoms after the manifestation of the disease, condition or disorder

As used herein, the term “white matter” relates to a component of the central nervous system, in the brain and superficial spinal cord, which consists mostly of glial cells and myelinated axons that transmit signals from one region of the cerebrum to another and between the cerebrum and lower brain centers.

One of the conditions resulting from age-related white matter loss, oligodendrocyte loss, or astrocyte loss in the brain which can be treated by the method of the present application is subcortical dimentia.

The glial progenitor cells may be introduced into the subject needing alleviation of the adverse effects by a variety of know techniques. These include, but are not limited to, injection, deposition, and grafting as described herein.

In one embodiment, the glial progenitor cells can be transplanted bilaterally into multiple sites of the subject, as described U.S. Pat. No. 7,524,491 to Goldman, Windrem et al., Cell Stem Cell 2:553-565 (2008), Han et al., Cell Stem Cell 12:342-353 (2013), and Wang et al., Cell Stem Cell 12:252-264 (2013), which are hereby incorporated by reference in their entirety). Methods for transplanting nerve tissues and cells into host brains are described by Bjorklund and Stenevi (eds), Neural Grafting in the Mammalian CNS, Ch. 3-8, Elsevier, Amsterdam (1985); U.S. Pat. No. 5,082,670 to Gage et al.; and U.S. Pat. No. 6,497,872 to Weiss et al., which are hereby incorporated by reference in their entirety. Typical procedures include intraparenchymal, intracallosal, intraventricular, intrathecal, and intravenous transplantation.

Intraparenchymal transplantation can be achieved by injection or deposition of tissue within the host brain so as to be apposed to the brain parenchyma at the time of transplantation. The two main procedures for intraparenchymal transplantation are: (1) injecting the donor cells within the host brain parenchyma or (2) preparing a cavity by surgical means to expose the host brain parenchyma and then depositing the graft into the cavity (Bjorklund and Stenevi (eds), Neural Grafting in the Mammalian CNS, Ch. 3, Elsevier, Amsterdam (1985), which is hereby incorporated by reference in its entirety). Both methods provide parenchymal apposition between the donor cells and host brain tissue at the time of grafting, and both facilitate anatomical integration between the graft and host brain tissue. This is of importance if it is required that the donor cells become an integral part of the host brain and survive for the life of the host.

Glial progenitor cells can also be delivered intracallosally as described in U.S. Patent Application Publication No. 20030223972 to Goldman, which is hereby incorporated by reference in its entirety. The glial progenitor cells can also be delivered directly to the forebrain subcortex, specifically into the anterior and posterior anlagen of the corpus callosum. Glial progenitor cells can also be delivered to the cerebellar peduncle white matter to gain access to the major cerebellar and brainstem tracts. Glial progenitor cells can also be delivered to the spinal cord.

Alternatively, the cells may be placed in a ventricle, e.g., a cerebral ventricle. Grafting cells in the ventricle may be accomplished by injection of the donor cells or by growing the cells in a substrate such as 30% collagen to form a plug of solid tissue which may then be implanted into the ventricle to prevent dislocation of the graft cells. For subdural grafting, the cells may be injected around the surface of the brain after making a slit in the dura.

Suitable techniques for cell delivery are described supra. In one embodiment, said preparation of glial progenitor cells is administered to the striatum, forebrain, brain stem, and/or cerebellum of the subject.

Delivery of the cells to the subject can include either a single step or a multiple step injection directly into the nervous system. For localized disorders such as demyelination of the optic nerve, a single injection can be used. Although adult and fetal oligodendrocyte precursor cells disperse widely within a transplant recipient's brain, for widespread disorders, multiple injections sites can be performed to optimize treatment. Injection is optionally directed into areas of the central nervous system such as white matter tracts like the corpus callosum (e.g., into the anterior and posterior anlagen), dorsal columns, cerebellar peduncles, cerebral peduncles. Such injections can be made unilaterally or bilaterally using precise localization methods such as stereotaxic surgery, optionally with accompanying imaging methods (e.g., high resolution MRI imaging). One of skill in the art recognizes that brain regions vary across species; however, one of skill in the art also recognizes comparable brain regions across mammalian species.

The cellular transplants can be optionally injected as dissociated cells but can also be provided by local placement of non-dissociated cells. In either case, the cellular transplants optionally comprise an acceptable solution. Such acceptable solutions include solutions that avoid undesirable biological activities and contamination. Suitable solutions include an appropriate amount of a pharmaceutically-acceptable salt to render the formulation isotonic. Examples of the pharmaceutically-acceptable solutions include, but are not limited to, saline, Ringer's solution, dextrose solution, and culture media. The pH of the solution is preferably from about 5 to about 8, and more preferably from about 7 to about 7.5.

The injection of the dissociated cellular transplant can be a streaming injection made across the entry path, the exit path, or both the entry and exit paths of the injection device (e.g., a cannula, a needle, or a tube). Automation can be used to provide a uniform entry and exit speed and an injection speed and volume.

The number of glial progenitor cells administered to the subject can range from about 10²-10⁸ at each administration (e.g., injection site), depending on the size and species of the recipient, and the volume of tissue requiring cell replacement. Single administration (e.g., injection) doses can span ranges of 10³-10⁵, 10⁴-10⁷, and 10⁵-10⁸ cells, or any amount in total for a transplant recipient patient.

Since the CNS is an immunologically privileged site, administered cells, including xenogeneic, can survive and, optionally, no immunosuppressant drugs or a typical regimen of immunosuppressant agents are used in the treatment methods. However, optionally, an immunosuppressant agent may also be administered to the subject. Immunosuppressant agents and their dosing regimens are known to one of skill in the art and include such agents as Azathioprine, Azathioprine Sodium, Cyclosporine, Daltroban, Gusperimus Trihydrochloride, Sirolimus, and Tacrolimus. Dosages ranges and duration of the regimen can be varied with the disorder being treated; the extent of rejection; the activity of the specific immunosuppressant employed; the age, body weight, general health, sex and diet of the subject; the time of administration; the route of administration; the rate of excretion of the specific immunosuppressant employed; the duration and frequency of the treatment; and drugs used in combination. One of skill in the art can determine acceptable dosages for and duration of immunosuppression. The dosage regimen can be adjusted by the individual physician in the event of any contraindications or change in the subject's status.

In one embodiment, one or more immunosuppressant agents can be administered to the subject starting at 10 weeks prior to cell administration. In one embodiment, the one or more immunosuppressant agents are administered to the subject starting at 9 weeks, 8 weeks, 7 weeks, 6 weeks, 5 weeks, 4 weeks, 3 weeks, 2 weeks, 1 week, 7 days, 6 days, 5 days, 4 days, 3 days, 2 days, 1 day, <24 hours prior to cell administration. In one embodiment, one or more immunosuppressant agents are administered to the subject starting on the day of cell administration and continuing for 1, 2, 3, 4, 5, 6, 7, 8, 9, 10, 11, 12 months post administration. In one embodiment, the one or more immunosuppressant agents are administered to the subject for >1 year following administration.

Suitable subjects for treatment in accordance with the methods described herein include any mammalian subject afflicted with age-related white matter loss. Exemplary mammalian subjects include humans, mice, rats, guinea pigs, and other small rodents, dogs, cats, sheep, goats, and monkeys. In one embodiment, the subject is human.

The above-described suppressor/rejuvenation therapy and cell therapy can be used together. For example, the inhibitory molecules, CRISPR/Cas systems, expression cassettes, or expression vectors described above can be used as therapeutic reagents in ex vivo applications. To that end, the reagents can be introduced into tissue or cells that are transplanted into a subject for therapeutic effect. The cells and/or tissue can be derived from an organism or subject that later receives the explant (e.g., isogenic or autologous), or can be derived from another organism or subject (e.g., a relative, a sibling, or a HLA matching donor) prior to transplantation (e.g., heterologous, xenogenic, allogeneic, or isogenic). The reagents can be used to modulate the expression of one or more genes in the cells or tissue, such that the cells or tissue obtain a desired phenotype or are able to perform a function when transplanted in vivo. In one embodiment, certain target cells from a patient are extracted or isolated. These isolated cells are contacted with the reagent targeting a specific nucleotide sequence within the cells under conditions suitable for uptake of the reagent by these cells (e.g., using delivery reagents such as cationic lipids, liposomes and the like or using techniques such as electroporation to facilitate the delivery of reagent into cells). The cells are then reintroduced back into the same patient or other patients.

For therapeutic applications, a pharmaceutically effective dose of the therapeutic reagent or pharmaceutical composition can be administered to the subject. A pharmaceutically effective dose is a dose required to prevent, inhibit the occurrence, or treat (alleviate a symptom to some extent, preferably all of the symptoms) of a disease state. One skilled in the art can readily determine a therapeutically effective dose of the reagent to be administer to a given subject, by taking into account factors, such as the size and weight of the subject, the extent of the disease progression or penetration, the age, health, and sex of the subject, the route of administration m and whether the administration is regional or systemic. Generally, an amount between 0.1 mg/kg and 100 mg/kg body weight/day of active ingredients is administered dependent upon potency of the negatively charged polymer. The therapeutic reagent or pharmaceutical composition can be administered in a single dose or in multiple doses.

Pharmaceutical Compositions

The present disclosure provides a pharmaceutical composition, or medicament, for preventing or treating an inherited or acquired disorder of myelin. In some embodiments, a pharmaceutical composition comprises one or more of the above-described protein molecule, polynucleotide, expression cassette, expression vector (e.g., viral vector genome, expression vector, rAAV vector), system (e.g., a CRISPR/Cas system or nucleic acid(s) encoding components of the system), and host cell.

The pharmaceutical composition further comprises a pharmaceutically-acceptable carrier, adjuvant, diluent, excipient and/or other medicinal agents. A pharmaceutically acceptable carrier, adjuvant, diluent, excipient or other medicinal agent is one that is not biologically or otherwise undesirable, e.g., the material may be administered to a subject without causing undesirable biological effects which outweigh the advantageous biological effects of the material. Any suitable pharmaceutically acceptable carrier or excipient can be used in the preparation of a pharmaceutical composition according to the invention (See e.g., Remington The Science and Practice of Pharmacy, Adeboye Adejare (Editor) Academic Press, November 2020).

A pharmaceutical composition is typically sterile, pyrogen-free and stable under the conditions of manufacture and storage. A pharmaceutical composition may be formulated as a solution (e.g., water, saline, dextrose solution, buffered solution, or other pharmaceutically sterile fluid), microemulsion, liposome, or other ordered structure suitable to accommodate a high product (e.g., viral vector particles, microparticles or nanoparticles) concentration.

In some embodiments, a pharmaceutical composition comprising the above-described protein, polynucleotide, expression cassette, expression vector, vector genome, host cell, or rAAV vector of the disclosure is formulated in water or a buffered saline solution. A carrier may be a solvent or dispersion medium containing, for example, water, ethanol, polyol (for example, glycerol, propylene glycol, and liquid polyethylene glycol, and the like), and suitable mixtures thereof. Proper fluidity can be maintained, for example, by use of a coating such as lecithin, by maintenance of a required particle size, in the case of dispersion, and by the use of surfactants. In some embodiments, it may be preferable to include isotonic agents, for example, a sugar, a polyalcohol such as mannitol, sorbitol, or sodium chloride in the composition. Prolonged adsorption of an injectable composition can be brought about by including, in the composition, an agent which delays absorption, e.g., a monostearate salt and gelatin. In some embodiments, a nucleic acid, vector and/or host cell of the disclosure may be administered in a controlled release formulation, for example, in a composition which includes a slow-release polymer or other carrier that protects the product against rapid release, including an implant and microencapsulated delivery system.

In some embodiments, a pharmaceutical composition of the disclosure is a parenteral pharmaceutical composition, including a composition suitable for intravenous, intraarterial, subcutaneous, intradermal, intraperitoneal, intramuscular, intraarticular, intraparenchymal (IP), intrathecal (IT), intracerebroventricular (ICV) and/or intracisternal magna (ICM) administration. In some embodiments, a pharmaceutical composition of this disclosure is formulated for administration by ICV injection. In some embodiments, a vector (e.g., a viral vector such as AAV) may be formulated in 350 mM NaCl and 5% D-sorbitol in PBS.

Methods of Administration

The above-described molecule, or polynucleotide, or vector (e.g., vector genome, rAAV vector), or system (e.g., a CRISPR/Cas systems or nucleic acid(s) encoding components of the system), or a cell may be administered to a subject (e.g., a patient) or a target cell in order to treat the subject. Administration of a vector to a human subject, or an animal in need thereof, can be by any means known in the art for administering a vector. Examples of a target cell include cells of the CNS, preferably oligodendrocytes, astrocytes, or the progenitor cells thereof.

A vector can be administered in addition to, and as an adjunct to, the standard of care treatment. That is, the vector can be co-administered with another agent, compound, drug, treatment or therapeutic regimen, either simultaneously, contemporaneously, or at a determined dosing interval as would be determined by one skilled in the art using routine methods. Uses disclosed herein include administration of an rAAV vector of the disclosure at the same time, in addition to and/or on a dosing schedule concurrent with, the standard of care for the disease as known in the art.

In some embodiments, a combination composition includes one or more immunosuppressive agents. In some embodiments, a combination composition includes an rAAV vector comprising a transgene (e.g., a polynucleotide encoding an RNA molecule disclosed herein) and one or more immunosuppressive agents. In some embodiments, a method includes administering or delivering an rAAV vector comprising the transgene to a subject and administering an immunosuppressive agent to the subject either prophylactically prior to administration of the vector, or after administration of the vector (i.e., either before or after symptoms of a response against the vector and/or the protein provided thereby are evident).

In one embodiment, a vector of the disclosure (e.g., an rAAV vector) is administered systemically. Exemplary methods of systemic administration include, but are not limited to, intravenous (e.g., portal vein), intraarterial (e.g., femoral artery, hepatic artery), intravascular, subcutaneous, intradermal, intraperitoneal, transmucosal, intrapulmonary, intralymphatic and intramuscular administration, and the like, as well as direct tissue or organ injection. One skilled in the art would appreciate that systemic administration can deliver a nucleic acid to all tissues. In some embodiments, direct tissue or organ administration includes administration to areas directly affected by oligodendrocyte deficiency (e.g., brain and/or central nervous system). In some embodiments, vectors of the disclosure, and pharmaceutical compositions thereof, are administered to the brain parenchyma (i.e., by intraparenchymal administration), to the spinal canal or the subarachnoid space so that it reaches the cerebrospinal fluid (CSF) (i.e., by intrathecal administration), to a ventricle of the brain (i.e., by intracerebroventricular administration) and/or to the cisterna magna of the brain (i.e., by intracisternal magna administration).

Accordingly, in some embodiments, a vector of the present disclosure is administered by direct injection into the brain (e.g., into the parenchyma, ventricle, cisterna magna, etc.) and/or into the CSF (e.g., into the spinal canal or subarachnoid space) to treat a disorder of myelin. A target cell of a vector of the present disclosure includes a cell located in the cortex, subcortical white matter of the corpus callosum, striatum and/or cerebellum. In some embodiments, a target cell of a vector of the present disclosure is an oligodendrocyte or a progenitor cell thereof. Additional routes of administration may also comprise local application of a vector under direct visualization, e.g., superficial cortical application, or other stereotaxic application.

In some embodiments, a vector of the disclosure is administered by at least two routes. For example, a vector is administered systemically and also directly into the brain. If administered via at least two routes, the administration of a vector can be, but need not be, simultaneous or contemporaneous. Instead, administration via different routes can be performed separately with an interval of time between each administration.

The above-described protein, or polynucleotide encoding the protein, or a vector genome, or a vector (e.g., an rAAV vector) comprising the polynucleotide may be used for transduction of a cell ex vivo or for administration directly to a subject (e.g., directly to the CNS of a patient with a disease). In some embodiments, a transduced cell (e.g., a host cell) is administered to a subject to treat or prevent a disease, disorder or condition (e.g., cell therapy for the disease). For example, an rAAV vector comprising a therapeutic nucleic acid (e.g., encoding a protein) can be preferably administered to an oligodendrocyte, an astrocyte, or a progenitor cell thereof in a biologically-effective amount.

The dosage amount of a vector depends upon, e.g., the mode of administration, disease or condition to be treated, the stage and/or aggressiveness of the disease, individual subject's condition (age, sex, weight, etc.), particular viral vector, stability of protein to be expressed, host immune response to the vector, and/or gene to be delivered. Generally, doses range from at least 1×10⁸, or more, e.g., 1×10⁹, 1×10¹⁰, 1×10¹¹, 1×10¹², 1×10¹³, 1×10¹⁴, 1×10¹⁵ or more vector genomes (vg) per kilogram (kg) of body weight of the subject to achieve a therapeutic effect.

In some embodiments, a polynucleotide encoding a protein described herein may be administered as a component of a DNA molecule (e.g., a recombinant nucleic acid) having a regulatory element (e.g., a promoter) appropriate for expression in a target cell (e.g., an oligodendrocyte, an astrocyte, or a progenitor cell thereof). The polynucleotide may be administered as a component of a plasmid or a viral vector, such as an rAAV vector. An rAAV vector may be administered in vivo by direct delivery of the vector (e.g., directly to the CNS) to a patient in need of treatment. An rAAV vector may be administered to a patient ex vivo by administration of the vector in vitro to a cell from a donor patient in need of treatment, followed by introduction of the transduced cell back into the donor (e.g., cell therapy).

Kit

The present disclosure provides a kit with packaging material and one or more components described therein. A kit typically includes a label or packaging insert including a description of the components or instructions for use in vitro, in vivo or ex vivo, of the components therein. A kit can contain a collection of such components, e.g., the above-described polynucleotide, nucleic acid, expression cassette, expression vector (e.g., viral vector genome, expression vector, rAAV vector), and host cell, and optionally a second active agent such as a compound, therapeutic agent, drug or composition.

A kit refers to a physical structure that contains one or more components of the kit. Packaging material can maintain the components in a sterile manner and can be made of material commonly used for such purposes (e.g., paper, glass, plastic, foil, ampules, vials, tubes, etc).

A label or insert can include identifying information of one or more components therein, dose amounts, clinical pharmacology of the active ingredients(s) including mechanism of action, pharmacokinetics and pharmacodynamics. A label or insert can include information identifying manufacture, lot numbers, manufacture location and date, expiration dates. A label or insert can include information on a disease (e.g., an inherited or acquired or age-related disorder of myelin such as HD) for which a kit component may be used. A label or insert can include instructions for a clinician or subject for using one or more of the kit components in a method, use or treatment protocol or therapeutic regimen. Instructions can include dosage amounts, frequency of duration and instructions for practicing any of the methods, uses, treatment protocols or prophylactic or therapeutic regimens described herein.

A label or insert can include information on potential adverse side effects, complications or reaction, such as a warning to a subject or clinician regarding situations where it would not be appropriate to use a particular composition.

### Definitions

Unless otherwise defined, all technical and scientific terms used herein have the meaning commonly understood by one of ordinary skill in the art to which this invention belongs. The terminology used herein is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise. The following terms have the meanings given:

As used herein, the term “about,” or “approximately” refers to a measurable value such as an amount of the biological activity, homology or length of a polynucleotide or polypeptide sequence, dose, time, temperature, and the like, and is meant to encompass variations of 25%, 20%, 19%, 18%, 17%, 16%, 15%, 14%, 13%, 12%, 11%, 10%, 9%, 8%, 7%, 6%, 5%, 4%, 3%, 2% 1%, 0.5% or even 0.1%, in either direction (greater than or less than) of the specified amount unless otherwise stated, otherwise evident from the context, or except where such number would exceed 100% of a possible value.

The term “transgene” refers to a heterologous polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome. In another aspect, it may be transcribed into a molecule that mediates RNA interference, such as miRNA, siRNA, or shRNA.

As used herein, the term “homologous,” or “homology,” refers to two or more reference entities (e.g., a nucleic acid or polypeptide sequence) that share at least partial identity over a given region or portion. For example, when an amino acid position in two peptides is occupied by identical amino acids, the peptides are homologous at that position. Notably, a homologous peptide will retain activity or function associated with the unmodified or reference peptide and the modified peptide will generally have an amino acid sequence “substantially homologous” with the amino acid sequence of the unmodified sequence. When referring to a polypeptide, nucleic acid or fragment thereof, “substantial homology” or “substantial similarity,” means that when optimally aligned with appropriate insertions or deletions with another polypeptide, nucleic acid (or its complementary strand) or fragment thereof, there is sequence identity in at least about 70% to 99% of the sequence. The extent of homology (identity) between two sequences can be ascertained using computer program or mathematical algorithm known in the art. Such algorithms that calculate percent sequence homology (or identity) generally account for sequence gaps and mismatches over the comparison region or area.

A nucleic acid or polynucleotide refers to a DNA molecule (e.g., a cDNA or genomic DNA), an RNA molecule (e.g., an mRNA), or a DNA or RNA analog. A DNA or RNA analog can be synthesized from nucleotide analogs. The nucleic acid molecule can be single-stranded or double-stranded, but preferably is double-stranded DNA.

An isolated or recombinant nucleic acid refers to a nucleic acid the structure of which is not identical to that of any naturally occurring nucleic acid or to that of any fragment of a naturally occurring genomic nucleic acid. The term therefore covers, for example, (a) a DNA which has the sequence of part of a naturally occurring genomic DNA molecule but is not flanked by both of the sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. The nucleic acid described above can be used to express the protein of this disclosure. For this purpose, one can operatively linked the nucleic acid to suitable regulatory sequences to generate an expression vector.

A “recombinant nucleic acid” is a combination of nucleic acid sequences that are joined together using recombinant technology and procedures used to join together nucleic acid sequences.

The terms “heterologous” DNA molecule and “heterologous” nucleic acid, as used herein, each refer to a molecule that originates from a source foreign to the particular host cell or, if from the same source, is modified from its original form. Thus, a heterologous gene in a host cell includes a gene that is endogenous to the particular host cell but has been modified through, for example, the use of shuffling or recombination. When used to describe two nucleic acid segments, the terms mean that the two nucleic acid segments are not from the same gene or, if form the same gene, one or both of them are modified from the original forms. The terms also include non-naturally occurring multiple copies of a naturally occurring DNA molecule. Thus, the terms refer to a nucleic acid segment that is foreign or heterologous to the cell, or homologous to the cell but in a position within the host cell nucleic acid in which the element is not ordinarily found. Exogenous DNA segments are expressed to yield exogenous RNAs or polypeptides. A “homologous DNA molecule” is a DNA molecule that is naturally associated with a host cell into which it is introduced.

A “regulatory sequence” includes promoters, enhancers, and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of protein or RNA desired, and the like. The expression vector can be introduced into host cells to produce an RNA or a polypeptide of interest. A promoter is defined as a DNA sequence that directs RNA polymerase to bind to DNA and initiate RNA synthesis. A strong promoter is one which causes RNAs to be initiated at high frequency.

A “promoter” is a nucleotide sequence which initiates and regulates transcription of a polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e.g., controls transcription or translation) segments of these regions.

“Operably linked” refers to an arrangement of elements wherein the components so described are configured so as to perform their usual function. Thus, a given promoter operably linked to a nucleic acid sequence is capable of effecting the expression of that sequence when the proper enzymes are present. The promoter need not be contiguous with the sequence, so long as it functions to direct the expression thereof. Thus, for example, intervening untranslated yet transcribed sequences can be present between the promoter sequence and the nucleic acid sequence and the promoter sequence can still be considered “operably linked” to the coding sequence. Thus, the term “operably linked” is intended to encompass any spacing or orientation of the promoter element and the DNA sequence of interest which allows for initiation of transcription of the DNA sequence of interest upon recognition of the promoter element by a transcription complex.

As used here, the term “genetic construct” or “nucleic acid construct,” refers to a non-naturally occurring nucleic acid molecule resulting from the use of recombinant DNA technology (e.g., a recombinant nucleic acid). A genetic or nucleic acid construct is a nucleic acid molecule, either single or double stranded, which has been modified to contain segments of nucleic acid sequences, which are combined and arranged in a manner not found in nature. A nucleic acid construct may be a “cassette” or a “vector” (e.g., a plasmid, an rAAV vector genome, an expression vector, etc.), that is, a nucleic acid molecule designed to deliver exogenously created DNA into a host cell.

“Expression cassette” as used herein means a nucleic acid sequence capable of directing expression of a particular nucleotide sequence in an appropriate host cell, which may include a promoter operably linked to the nucleotide sequence of interest that may be operably linked to termination signals. It also may include sequences required for proper translation of the nucleotide sequence. The coding region usually codes for an RNA or protein of interest. The expression cassette including the nucleotide sequence of interest may be chimeric. The expression cassette may also be one that is naturally occurring but has been obtained in a recombinant form useful for heterologous expression. The expression of the nucleotide sequence in the expression cassette may be under the control of a constitutive promoter or of a regulatable promoter that initiates transcription only when the host cell is exposed to some particular stimulus. In the case of a multicellular organism, the promoter can also be specific to a particular tissue or organ or stage of development.

A vector refers to a nucleic acid molecule capable of transporting another nucleic acid to which it has been linked. The vector may or may not be capable of autonomous replication or integrate into a host DNA. Examples include a plasmid, virus (e.g., an rAAV), cosmid, or other vehicle that can be manipulated by insertion or incorporation of a nucleic acid (e.g., a recombinant nucleic acid). A vector can be used for various purposes including, e.g., genetic manipulation (e.g., cloning vector), to introduce/transfer a nucleic acid into a cell, to transcribe or translate an inserted nucleic acid in a cell. In some embodiments a vector nucleic acid sequence contains at least an origin of replication for propagation in a cell. In some embodiments, a vector nucleic acid includes a heterologous nucleic acid sequence, an expression control element(s) (e.g., promoter, enhancer), a selectable marker (e.g., antibiotic resistance), a poly-adenosine (polyA) sequence and/or an ITR. In some embodiments, when delivered to a host cell, the nucleic acid sequence is propagated. In some embodiments, when delivered to a host cell, either in vitro or in vivo, the cell expresses the polypeptide encoded by the heterologous nucleic acid sequence. In some embodiments, when delivered to a host cell, the nucleic acid sequence, or a portion of the nucleic acid sequence is packaged into a capsid. A host cell may be an isolated cell or a cell within a host organism. In addition to a nucleic acid sequence (e.g., transgene) which encodes an RNA, or a polypeptide or a protein, additional sequences (e.g., regulatory sequences) may be present within the same vector (i.e., in cis to the gene) and flank the gene. In some embodiments, regulatory sequences may be present on a separate (e.g., a second) vector which acts in trans to regulate the expression of the gene. Plasmid vectors may be referred to herein as “expression vectors.”

As used herein, the term “vector genome” refers to a recombinant nucleic acid sequence that is packaged or encapsidated to form an rAAV vector. Typically, a vector genome includes a heterologous polynucleotide sequence, e.g., a transgene, regulatory elements, ITRs not originally present in the capsid. In cases where a recombinant plasmid is used to construct or manufacture a recombinant vector (e.g., rAAV vector), the vector genome does not include the entire plasmid but rather only the sequence intended for delivery by the viral vector. This non-vector genome portion of the recombinant plasmid is typically referred to as the “plasmid backbone,” which is important for cloning. selection and amplification of the plasmid, a process that is needed for propagation of recombinant viral vector production, but which is not itself packaged or encapsidated into an rAAV vector.

As used herein, the term “viral vector” generally refers to a viral particle that functions as a nucleic acid delivery vehicle and which comprises a vector genome (e.g., comprising a transgene instead of a nucleic acid encoding an AAV rep and cap) packaged within the viral particle (i.e., capsid) and includes, for example, lenti- and parvo-viruses, including AAV serotypes and variants (e.g., rAAV vectors). A recombinant viral vector does not comprise a vector genome comprising a rep and/or a cap gene.

As used herein, the term “overexpressing,” “overexpress,” “overexpressed,” or “overexpression,” when referring to the production of a nucleic acid or a protein in a host cell means that the nucleic acid or protein is produced in greater amounts than it is produced in its naturally occurring environment. It is intended that the term encompass overexpression of endogenous, as well as exogenous or heterologous nucleic acids and proteins. As such, the terms and the like are intended to encompass increasing the expression of a nucleic acid or a protein in a cell to a level greater than that the cell naturally contains. In certain embodiments, the expression level or amount of the nucleic acid or protein in a cell is increased by at least 5%, 10%, 20%, 25%, 30%, 40%, 50%, 60%, 70%, 75%, 80%, 85%, 90%, 95%, 100%, 110%, 120%, 130%, 140%, 150%, 160%, 170%, 180%, 190%, 200%, 250%, 300%, 350%, 400%, 450%, 500%, 550%, 600%, 650%, 700%, 750%, 800%, 850%, 900%, 950%, or 1000% as compared to the level or amount that the cell naturally contains.

In the context of a mutant or diseased cell, the terms “overexpressing,” “overexpress,” “overexpressed,” and “overexpression,” and the like are intended to encompass increasing the expression of a nucleic acid or a protein to a level greater than that a mutant cell, a diseased cell, a wildtype cell, or a non-diseased cell contains. In certain embodiments, the expression level or amount of the nucleic acid or protein in a mutant or diseased cell is increased by at least 5%, 10%, 20%, 25%, 30%, 40%, 50%, 60%, 70%, 75%, 80%, 85%, 90%, 95%, 100%, 110%, 120%, 130%, 140%, 150%, 160%, 170%, 180%, 190%, 200%, 250%, 300%, 350%, 400%, 450%, 500%, 550%, 600%, 650%, 700%, 750%, 800%, 850%, 900%, 950%, or 1000% as compared to the level or amount that a mutant cell, a diseased cell, a wildtype cell, or a non-diseased cell contains.

“Anti-sense” refers to a nucleic acid sequence, regardless of length, that is complementary to the coding strand or mRNA of a nucleic acid sequence. Antisense RNA can be introduced to an individual cell, tissue or organanoid. An anti-sense nucleic acid can contain a modified backbone, for example, phosphorothioate, phosphorodithioate, or other modified backbones known in the art, or may contain non-natural internucleoside linkages.

As referred to herein, a “complementary nucleic acid sequence” is a nucleic acid sequence capable of hybridizing with another nucleic acid sequence comprised of complementary nucleotide base pairs. By “hybridize” is meant pair to form a double-stranded molecule between complementary nucleotide bases (e.g., adenine (A) forms a base pair with thymine (T), as does guanine (G) with cytosine (C) in DNA) under suitable conditions of stringency. (See, e.g., Wahl, G. M. and S. L. Berger (1987) Methods Enzymol. 152:399; Kimmel, A. R. (1987) Methods Enzymol. 152:507).

A “suppressor” or an “inhibitor” refers to an agent that causes a decrease in the expression or activity of a target gene or protein, respectively.

The terms “inhibit”, “down-regulate”, or “reduce”, refer to the reduction in the expression of a gene, or level of RNA molecules or equivalent RNA molecules encoding one or more proteins or protein subunits, or activity of one or more proteins or protein subunits, below that observed in the absence of an inhibitor, suppressor or repressor, such as the inhibitory nucleic acid molecules (e.g., siRNA) described herein. Down-regulation can be associated with post-transcriptional silencing, such as, RNAi mediated cleavage or by alteration in DNA methylation patterns or DNA chromatin structure.

As used herein, an “inhibitory nucleic acid” is a double-stranded RNA, RNA interference, miRNA, siRNA, shRNA, or antisense RNA, or a portion thereof, or a mimetic thereof, that when administered to a mammalian cell results in a decrease in the expression of a target gene. Typically, a nucleic acid inhibitor comprises at least a portion of a target nucleic acid molecule, or an ortholog thereof, or comprises at least a portion of the complementary strand of a target nucleic acid molecule. Typically, expression of a target gene is reduced by 10%, 25%, 50%, 75%, or even 90-100%.

As used herein, the term “siRNA” intends a double-stranded RNA molecule that interferes with the expression of a specific gene or genes post-transcription. In some embodiments, the siRNA functions to interfere with or inhibit gene expression using the RNA interference pathway. Similar interfering or inhibiting effects may be achieved with one or more of short hairpin RNA (shRNA), microRNA (mRNA) and/or nucleic acids (such as siRNA, shRNA, or miRNA) comprising one or more modified nucleic acid residue—e.g. peptide nucleic acids (PNA), locked nucleic acids (LNA), unlocked nucleic acids (UNA), or triazole-linked DNA. Optimally, a siRNA is 18, 19, 20, 21, 22, 23 or 24 nucleotides in length and has a 2-base overhang at its 3′ end. These dsRNAs can be introduced to an individual cell or culture system. Such siRNAs are used to downregulate mRNA levels or promoter activity.

As used herein, the terms “treat,” “treating” or “treatment” refer to administration of a therapy that partially or completely alleviates, ameliorates, relieves, inhibits, delays onset of, reduces severity of, and/or reduces incidence of one or more symptoms, features, and/or causes of a particular disease, disorder, and/or condition.

As used herein, the term “ameliorate” means a detectable or measurable improvement in a subject's disease, disorder or condition, or symptom thereof, or an underlying cellular response. A detectable or measurable improvement includes a subjective or objective decrease, reduction, inhibition, suppression, limit or control in the occurrence, frequency, severity, progression or duration of, complication cause by or associated with, improvement in a symptom of, or a reversal of a disease, disorder or condition.

As used herein, the term “associated with” refers to with one another, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population).

As used herein, the term “prevent” or “prevention” refers to delay of onset, and/or reduction in frequency and/or severity of one or more sign or symptom of a particular disease, disorder or condition (e.g., a myelin disease). In some embodiments, prevention is assessed on a population basis such that an agent is considered to “prevent” a particular disease, disorder or condition if a statistically significant decrease in the development, frequency and/or intensity of one or more sign or symptom of the disease, disorder or condition is observed in a population susceptible to the disease, disorder or condition. Prevention may be considered complete when onset of disease, disorder or condition has been delayed for a predefined period of time.

As used herein, the term “therapeutically effective amount” refers to an amount that produces the desired therapeutic effect for which it is administered. In some embodiments, the term refers to an amount that is sufficient, when administered to a population suffering from or susceptible to a disease, disorder or condition in accordance with a therapeutic dosing regimen, to treat the disease, disorder or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment.

“Population” of cells refers to any number of cells greater than 1, but is at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

As used herein, the term “stem cells” refers to cells with the ability to both replace themselves and to differentiate into more specialized cells. Their self-renewal capacity generally endures for the lifespan of the organism. A pluripotent stem cell can give rise to all the various cell types of the body. A multipotent stem cell can give rise to a limited subset of cell types. For example, a hematopoietic stem cell can give rise to the various types of cells found in blood, but not to other types of cells. Multipotent stem cells can also be referred to as somatic stem cells, tissue stem cells, lineage-specific stem cells, and adult stem cells. The non-stem cell progeny of multipotent stem cells are progenitor cells (also referred to as restricted-progenitor cells). Progenitor cells give rise to fully differentiated cells, but a more restricted set of cell types than stem cells. Progenitor cells also have comparatively limited self-renewal capacity; as they divide and differentiate they are eventually exhausted and replaced by new progenitor cells derived from their upstream multipotent stem cell.

“Induced pluripotent stem cells,” commonly abbreviated as iPS cells or iPSCs, refer to a type of pluripotent stem cell artificially prepared from a non-pluripotent cell, typically an adult somatic cell, or terminally differentiated cell, such as fibroblast, a hematopoietic cell, a myocyte, a neuron, an epidermal cell, or the like, by introducing certain factors, referred to as reprogramming factors.

“Pluripotency” refers to a stem cell that has the potential to differentiate into all cells constituting one or more tissues or organs, or particularly, any of the three germ layers: endoderm (interior stomach lining, gastrointestinal tract, the lungs), mesoderm (muscle, bone, blood, urogenital), or ectoderm (epidermal tissues and nervous system). “Pluripotent stem cells” used herein refer to cells that can differentiate into cells derived from any of the three germ layers, for example, direct descendants of totipotent cells or induced pluripotent cells.

As used herein, “therapeutic cells” refers to a cell population that ameliorates a condition, disease, and/or injury in a patient. Therapeutic cells may be autologous (i.e., derived from the patient), allogeneic (i.e., derived from an individual of the same species that is different from the patient) or xenogeneic (i.e., derived from a different species than the patient). Therapeutic cells may be homogenous (i.e., consisting of a single cell type) or heterogeneous (i.e., consisting of multiple cell types). The term “therapeutic cell” includes both therapeutically active cells as well as progenitor cells capable of differentiating into a therapeutically active cell.

The term “autologous” refers to any material derived from the same subject or individual to which it is later to be re-introduced. For example, the autologous cell therapy method described herein involves collection of glial cells, or progenitors thereof from a donor, e.g., a patient, which are then engineered to express, e.g., a transgene, and then administered back to the same donor, e.g., patient.

The term “heterologous” refers to any material (e.g., cells or tissue scaffold) derived from a different subject or individual. As used herein, “heterologous” or “non-endogenous” or “exogenous” also refers to any material (e.g., gene, protein, compound, molecule, cell, or tissue or tissue component) or activity that is not native to a host cell or a host subject, or is any gene, protein, compound, molecule, cell, tissue or tissue component, or activity native to a host or host cell but has been altered or mutated such that the structure, activity or both is different as between the native and mutated versions.

The term “allogeneic” refers to any material (e.g., cells or tissue) derived from one individual which is then introduced to another individual of the same species, e.g., allogeneic cell transplantation. For example, cells may be obtained from a first subject, modified ex vivo according to the methods described herein and then administered to a second subject in order to treat a disease. In such embodiments, the cells administered to the subject are allogeneic and heterologous cells.

The term “xenogenic” refers to any material (e.g., cells or tissue) derived from an individual of a different species.

The term “isogenic” refers to any materials (e.g., cells or tissue) characterized by essentially identical genes.

As used herein, the term “subject” refers to an organism, for example, a mammal (e.g., a human, a non-human mammal, a non-human primate, a primate, a laboratory animal, a mouse, a rat, a hamster, a gerbil, a cat, a dog). In some embodiments, a subject is a non-human disease model. In some embodiments, a human subject is an adult, adolescent, or pediatric subject. In some embodiments, a subject is suffering from a disease, disorder or condition, e.g., a disease, disorder or condition that can be treated as provided herein. In some embodiments, a subject is suffering from a disease, disorder or condition associated with deficient or dysfunctional myelin. In some embodiments, a subject is susceptible to a disease, disorder, or condition. In some embodiments, a susceptible subject is predisposed to and/or shows an increased risk (as compared to the average risk observed in a reference subject or population) of developing a disease, disorder or condition. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a human patient. In some embodiments, a subject is an individual to whom diagnosis and/or therapy is and/or has been administered.

As used herein, the term “therapeutically effective amount” refers to an amount that produces the desired therapeutic effect for which it is administered. In some embodiments, the term refers to an amount that is sufficient, when administered to a population suffering from or susceptible to a disease, disorder or condition in accordance with a therapeutic dosing regimen, to treat the disease, disorder or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment.

The examples below are intended to exemplify the practice of embodiments of the disclosure but are by no means intended to limit the scope thereof. Examples Part A relates to competitive replacement of glial cells. Examples Part B relates to rejuvenation of glial progenitor cells.

### EXAMPLES

### Examples Part A: Competitive Replacement Of Glial Progenitor Cells In Adult Brain

Materials and Methods

Human Embryonic Stem Cell Lines and Culture Conditions

Sibling human embryonic stem cells (hESCs) lines GENEA019 (WT: 18; 15 CAG) and GENEA020 (HD: 48; 17 CAG). hESC were regularly cultured under feeder-free conditions on 0.55 ug/cm² human recombinant laminin 521 (BIOLAMINA, cat. no. LN521) coated cell culture flasks with mTeSR1 medium (STEMCELL TECHNOLOGIES, cat. no. 85850). Daily medium changes were performed. hESCs were routinely passaged at 80% confluency onto freshly coated flasks. Passaging was performed using ReLeSR (STEMCELL TECHNOLOGIES, cat. no. 05872). All hESCs and differentiated cultures were maintained in a 5% CO2 incubator at 37° C. and routinely checked for contamination and mycoplasma free status.

Generation of Fluorescent Reporter hESCs

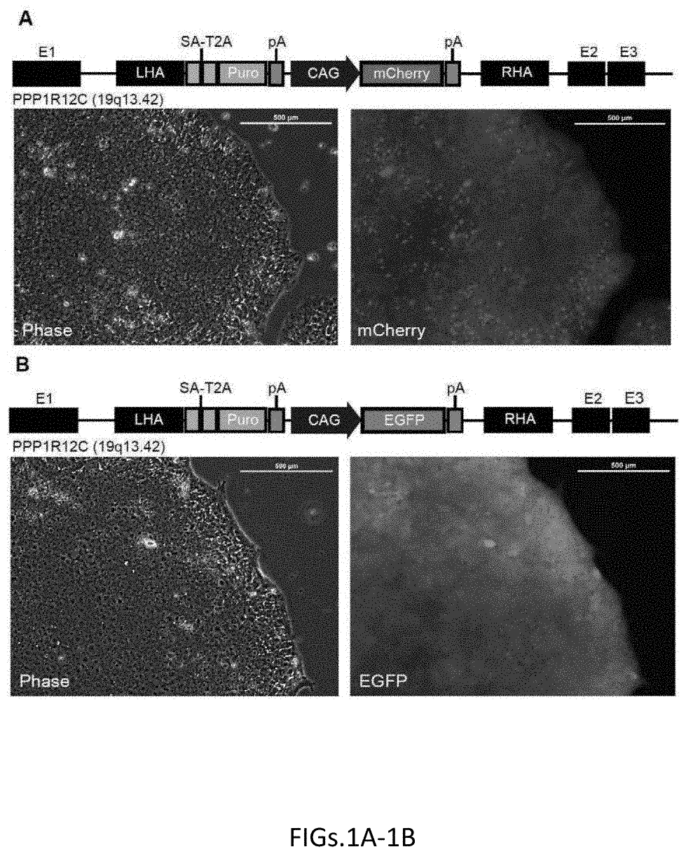

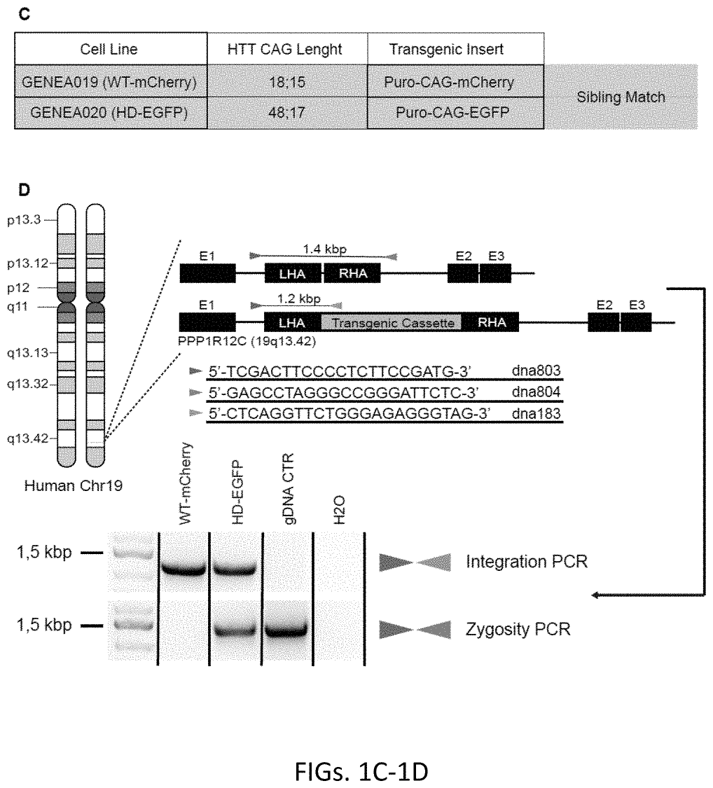

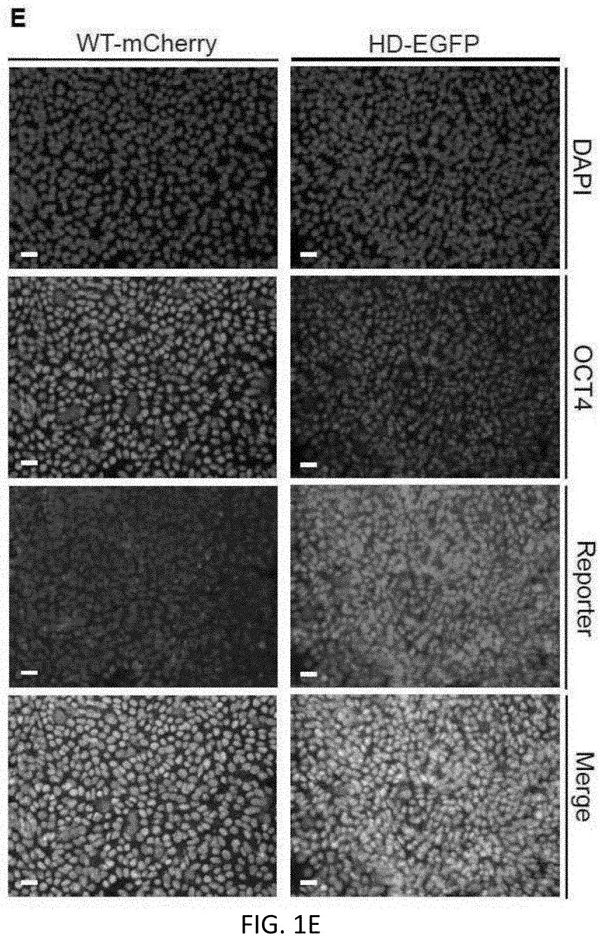

For ubiquitous and distinct fluorescent labeling of WT and HD cells (FIG. 1), reporter constructs driving expression of either mCherry or EGFP were inserted into the AAVS1 safe-harbor locus of WT GENEA019 and HD GENEA020 hESCs, respectively, using a modified version of the CRISPR-Cas9 mediated strategy previously described in Oceguera-Yanez, F., et al., Methods 101, 43-55, which is hereby incorporated by reference in its entirety). To prepare hESCs for plasmid delivery by electroporation, hESC were harvested as single cell suspension following dissociation with Accutase (StemCell Technologies, cat. no. 07920), washed in culture medium, and counted with the automated cell counter NucleoCounter NC-200 (ChemoMetec). Per electroporation, a total of 1.5×10⁶ cells were mixed with 5 μg of the AAVS1 targeting CRISPR-Cas9 plasmid (pXAT2) and 5 μg of reporter donor plasmid (pAAVS1-P-CAG-mCh or pAAVS1-P-CAG-GFP). pXAT2 (Addgene plasmid no. 80494), pAAVS1-P-CAG-mCh (Addgene plasmid no. 80491) and pAAVS1-P-CAG-GFP (Addgene plasmid no. 80492) were a gift from Knut Woltjen. Electroporation was performed using an Amaxa 4D-Nucleofector (Lonza) with the P3 primary cell kit (Lonza, cat. no. V4XP-3024) according to manufacturer's guidelines. After nucleofection, the electroporated hESC suspensions were transferred to 10 cm cell culture dishes and cultured with mTeSR1 supplemented with 10 μM Y-27632 (Tocris, cat. no. 1254) for the first 24h. Electroporated hESCs were grown for 48-72h and then treated with 0.5 μg/μL puromycin (ThermoFisher, cat. no. A1113803). Electroporated hESC cultures were kept under puromycin until individual colonies were large enough to be picked manually. Colonies were assessed by fluorescent microscopy and transferred to a 96-well plate based on uniformity of fluorescent protein expression. Following their expansion, each clone was split for further expansion and for genotyping. For genotyping, DNA was extracted using the prepGEM Tissue DNA extraction kit (Zygem). Correctly targeted transgenic integrations in the AAVS1 locus were detected by PCR using the following primers: dna803: TCGACTTCCCCTCTTCCGATG (SEQ ID NO; 1) and dna804: CTCAGGTTCTGGGAGAGGGTAG (SEQ ID NO; 2); while the zygosity of the integrations was determined by the presence or absence of a WT allele using an additional primer: (dna803 and dna183: GAGCCTAGGGCCGGGATTCTC(SEQ ID NO; 3)). hESC clones with correctly targeted insertions were cryopreserved with Pro-Freeze CDM medium (Lonza, cat. no. BEBP12-769E) and expanded for karyotyping and array comparative genomic hybridization (aCGH) characterization prior to experimental application.

Karyotyping and aCGH

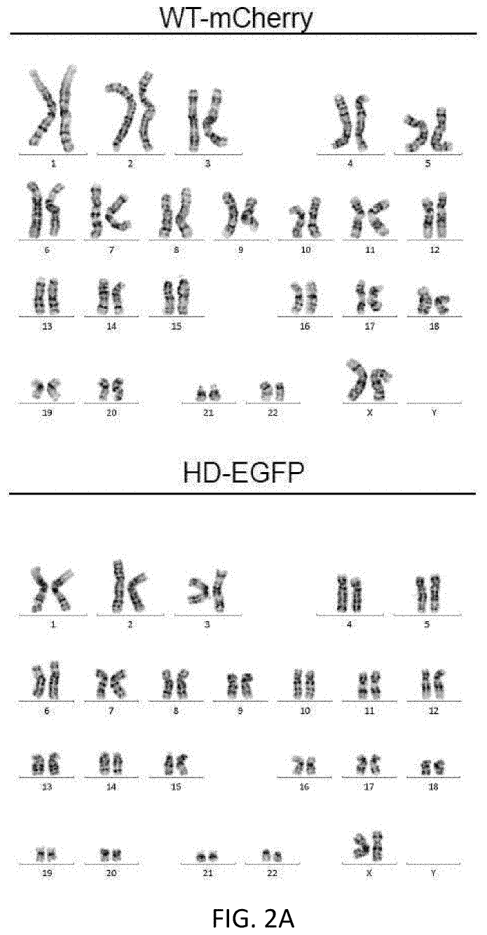

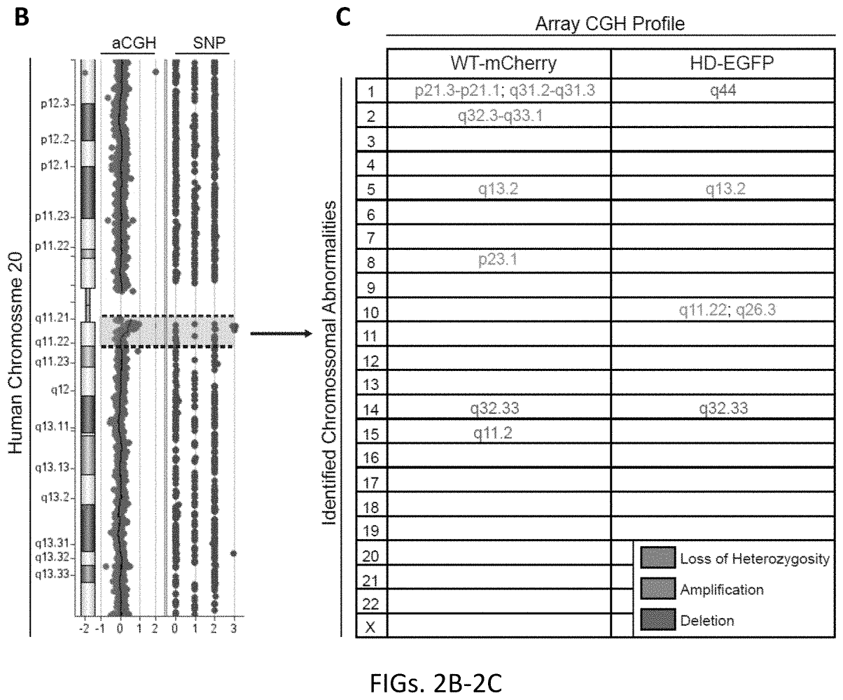

The karyogram of generated reporter hESC lines was analyzed on metaphase spreads by G-banding (Institut für Medizinishche Genetik and Angewandte Genomik, Universitäsklinikum Tubingen). All hESC lines used in this study harbor a normal karyotype. Additionally, acquired copy number variants (CNVs) and loss-of-heterozygosity regions (LOH) were assessed by aCGH (Cell Line Genetics). A variety of CNVs and LOH within and outside of normal range were identified (FIG. 2), but none that are expected to influence the outcomes of competitive interactions between the clones.

Derivation of hGPCs from Reporter WT and HD hESCs

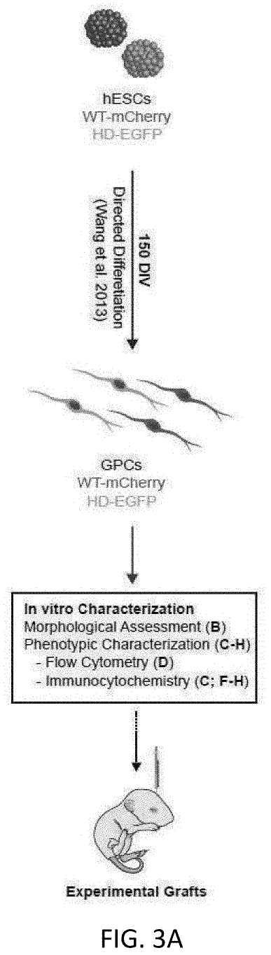

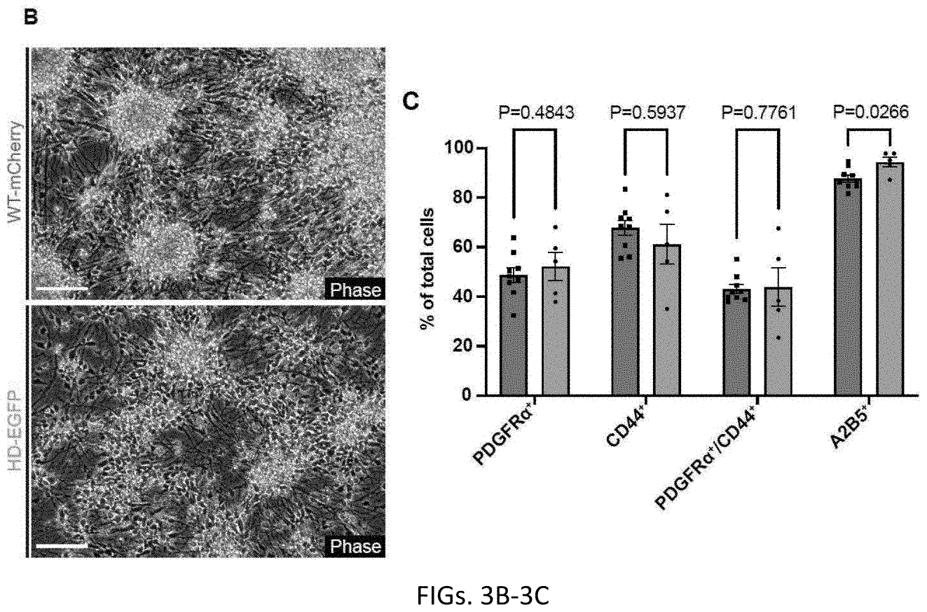

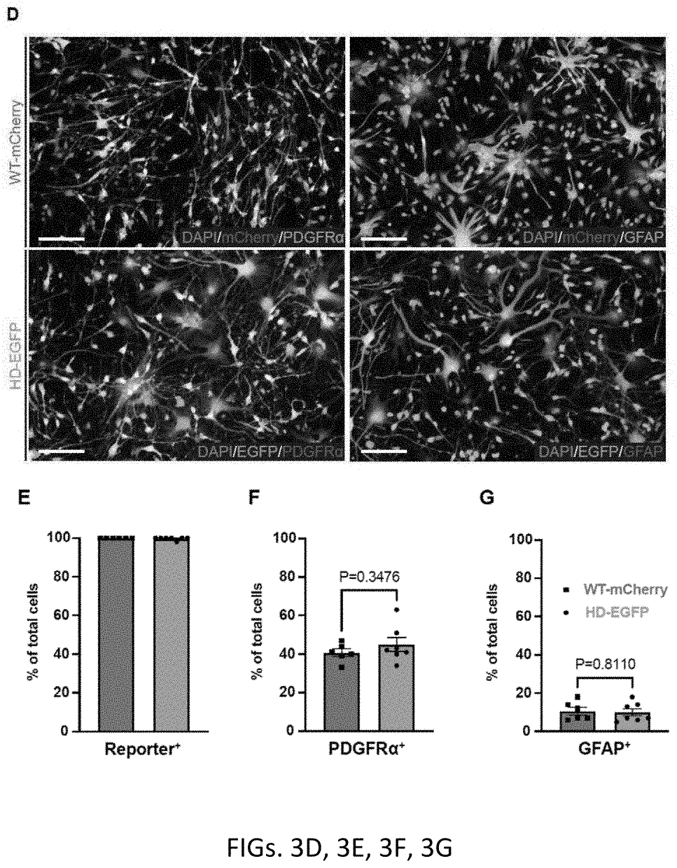

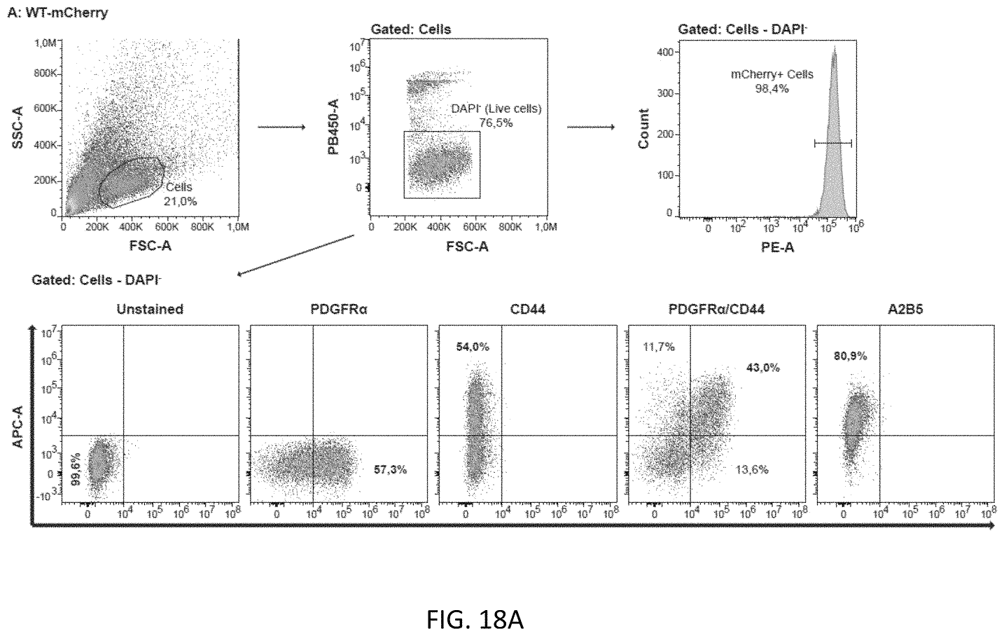

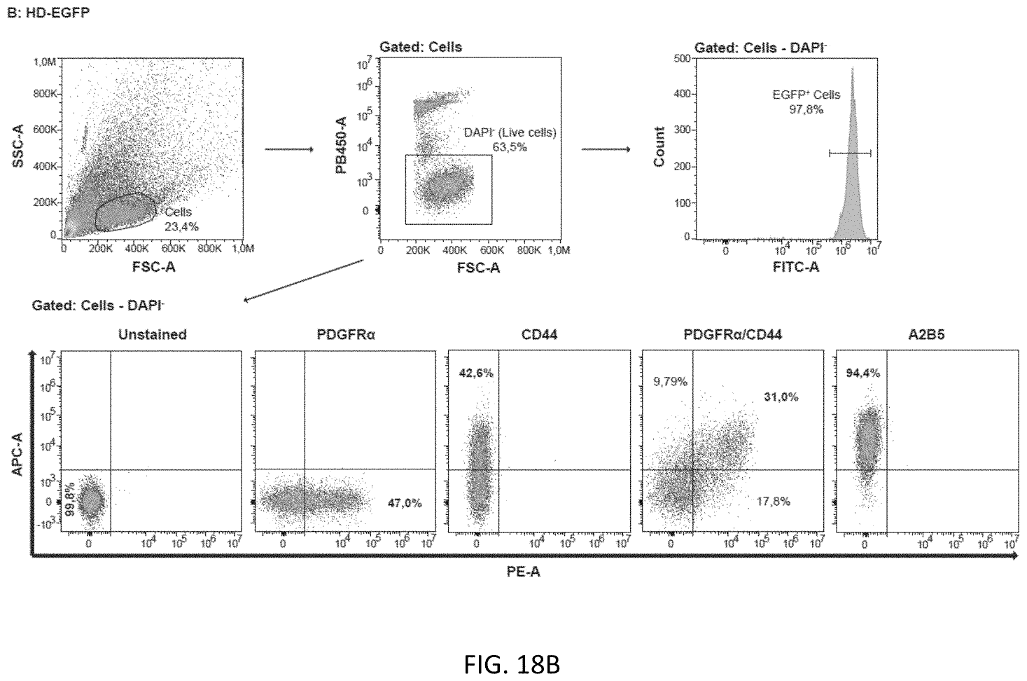

Human GPCs were derived from both reporter WT and HD hESCs using applicants' well-established protocol (Wang et al., Cell Stem Cell 12, 252-264. which is hereby incorporated by reference in its entirety), with minor modifications to the embryoid body (EB) generation step. Details on the EB generation step are included in the supplementary information. Cells were collected for xenotransplantation between 150 and 200 DIV, at which time the cultures derived from both WT-mCherry and HD-EGFP hESCs were rich in PDGFRα⁺/CD44⁺ bipotential glial progenitor cells. A detailed characterization of the generated cultures by flow cytometry and immunocytochemistry can be found in FIG. 3 and FIGS. 18A and 18B.

Cell Preparation for Xenotransplantation

To prepare cells for xenotransplantation, glial cultures were collected in Ca²⁺/Mg²⁺-free Hanks' balanced salt solution (HBSS^{(-/-)}; THERMOFISHER, cat. no. 14170112), mechanically dissociated to small clusters by gentle pipetting and counted with a hemocytometer. The cell suspension was then spun and resuspended in cold HBSS^{(-/-)} at a final concentration of 10⁵ cells/μL and kept on ice until transplanted.

Hosts and Xenotransplantation Paradigms

In vivo modelling of human glial striatal repopulation: To generate human-mouse chimeras harboring mHtt-expressing human glia (HD chimeras), newborn immunocompromised Rag1^{(-/-)} pups (Mombaerts, P. et al, Cell 68, 869-877. 10.1016/0092-8674(92)90030-g, which is hereby incorporated by reference in its entirety) were cryoanesthetized, secured in a custom baked clay stage, and injected bilaterally with 100,000 HD-EGFP glia (50,000 per hemisphere) into the presumptive striatum within 48h from birth. Cells were delivered using a 10 μL syringe (HAMILTON, cat. no. 7653-01) with pulled glass pipettes at a depth of 1.2 to 1.4 mm. The pups were then returned to their mother, until weaned. To model human glial striatal repopulation, 36 weeks old HD chimeras were anesthetized by ketamine/xylazine and secured in a stereotaxic frame. 200,000 WT glia were delivered bilaterally using a 10 μL syringe and metal needle into the humanized striatum (AP: +0.8 mm; ML: ±1.8 mm; DV: −2.5 to −2.8 mm). To minimize damage, cells were infused at a controlled rate of 175 nL/min using a controlled micropump system (World Precision Instruments). Backflow was prevented by leaving the needle in place for an additional 5 min. Experimental animals were compared to HD chimeric littermates that did not receive WT glia and to non-chimeric Rag1^{(-/-)} mice that received WT glia at 36 weeks of age following this exact procedure.

Neonatal Striatal Co-Engraftments

To model the cell-intrinsic effects of mHtt-expression on the outcomes of competition between human glia, newborn Rag1^{(-/-)} mice were injected following the same neonatal striatal xenotransplant protocol above described, but instead a total of 200,000 human glia (100,000 per hemisphere) composed of a 1:1 mixture of glia derived from WT-mCherry and HD-EGFP hESCs were delivered. Control littermates received injections composed of either WT-mCherry or HD-EGFP human glia.

Aseptic technique was used for all xenotransplants. All mice were housed in a pathogen-free environment, with ad libitum access to food and water, and all procedures were performed in agreement with protocols approved by the University of Rochester Committee on Animal Resources.

Tissue Processing

Experimental animals were perfused with HBSS^{(-/-)} followed by 4% PFA. The brains were removed, post-fixed for 2h in 4% PFA and rinsed 3× with PBS. They were then incubated in 30% sucrose solution (SIGMA-ALDRICH, cat. no. 59378) until equilibrated at which point, they were embedded in OCT in a sagittal orientation (Sakura, cat. no. 4583), frozen in 2-methylbutane (Fisher Scientific, cat. no. 11914421) at temperatures between −60 and −70° C. and transferred to a−80° C. freezer. The resulting blocks were then cut in 20 μm sections on a CM1950 cryostat (Leica), serially collected on adhesion slides and stored at −20° C. until further use.

Immunostaining

Phenotyping of human cells was accomplished by immunostaining for their respective fluorescent reporter, together with a specific phenotype marker: Olig2 (oligodendrocyte transcription factor, marking GPCs) and GFAP (glial fibrillary acidic protein, marking astrocytes). Fluorescent reporters were used as makers for human cells as their expression remained ubiquitous throughout the animal's life (FIG. 4). In animals that received a 1:1 mixture of WT-mCherry and WT-untagged human glia, the latter were identified by the expression of human nuclear antigen and the lack of fluorescent reporter expression. To immunolabel, sections were rehydrated with PBS, then permeabilized and blocked using a permeabilization/blocking buffer (PBS+0.1% Triton-X (SIGMA-ALDRICH cat. no. T8787)+10% Normal Goat Serum (THERMOFISHER, cat. no. 16210072)) for 2h. Sections were then incubated overnight with primary antibodies targeting phenotypic makers at 4° C. The following day, the primary antibodies were thoroughly rinsed from the sections with PBS and secondary antibodies were applied to the sections for 1h. After thoroughly rinsing out the secondary antibodies with PBS, a second round of primary antibodies, this time against fluorescent reporters, were applied to the sections overnight at 4° C. These were rinsed with PBS the following day and the sections were incubated with secondary antibodies for 1h. The slides were again thoroughly washed with PBS and mounted with VECTASHIELD VIBRANCE (Vector Labs, cat. no. H-1800).

Xenotransplant Mapping and 3D Reconstruction

To map human cell distribution within the murine striatum, whole brain montages of equidistantly spaced 160 μm apart sagittal sections spanning the entire striatum were captured using a NIKON NI-E ECLIPSE microscope equipped with a DS-Fi1 camera at 10× magnification and processed in the NIS-Elements imaging software (NIKON). The striatum within each section was outlined and immunolabeled human cells were identified and mapped within the outlined striatum using the Stereo Investigator software (MICROBRIGHTFIELD Bioscience). When applicable, the injection site for WT glia was mapped as a reference point for further volumetric quantification of human cell distribution. Mapped sections were then aligned using the lateral ventricle as a reference to produce a 3D reconstructed model of the humanized murine striatum. After 3D reconstruction, the cartesian coordinates for each human cell marker, injection site and striatal outlines were exported for further analysis.

To assess the distribution and proportion of proliferative cells in each human cell population within the striatum, immunolabeled human cells expressing Ki67 were mapped in every third section of the 15 sections when performing the 3D reconstructions.

Volumetric Quantification

To quantify the spatial distribution of HD glia in HD chimeras, the volumes for each quantified striatal section were calculated by multiplying the section thickness (20 μm) by the section area. The cell density for each section was then calculated by dividing the number of marked cells in each section by their respective volume.

To quantify the spatial-temporal dynamics of competing WT and HD glia, a program was developed to calculate the volumetric distribution of each cell population as a function of distance to the WT glia delivery site in 3D reconstructed datasets (FIG. 4). To that end, each quantified section was given an upper and lower boundary zᵤzₗ, by representing the striatal outline as two identical polygons separated from each other by the section thickness (20 μm). Then, since the depth-wise location of each cell marker within each individual section is unknown, marked cells within each section were represented as uniform point probability functions with constant probability across the section. I.e., each cell marker in a section from zₗ to zᵤ has a probability function:

$$P ( z ) = \{ \begin{matrix}\frac{1}{{z}_{u} - {z}_{l}} , & {z}_{l} \leq z < {z}_{u} \\ 0 , & otherwise\end{matrix} .$$

The spatial distribution of each cell population was then measured by counting the number of marked cells within concentric spherical shells radiating from the WT glia delivery site in radial increments of 125 μm (For control HD chimeras, an average of the coordinates of the WT glia delivery site was used). Marked cells were counted if their respective representative line segments are fully inside, fully outside or intersecting the spherical shell at either the upper or lower boundary. The density of each cell population ρ_{a,b}—where a,b represents the minimum and maximum radii of the spherical shell—was then calculated by dividing number of marked cells within the spherical shell by the combined section volume within the shell:

ρ_{a,b}=N_{a,b}/V_{a,b}

where N_{a,b} is the sum of integrated point probability functions over each section for each point and V_{a,b} is the combined section volume within the spherical shell. Subsequent analyses were restricted to a 2 mm spherical radius. The code was implemented in Python 3.8 and the package Shapely 1.7 to represent polygons and calculate circle intersections of the polygons.

Stereological Estimations and Phenotyping

Estimations of the total amount of human cells and their respective phenotyping were performed stereologically using the optical fractionator method (West, M. J. (1999). Trends in Neurosciences 22, 51-61, which is hereby incorporated by reference in its entirety) in 5 equidistantly separated 480 μm apart sections spanning the entire striatum. First, whole striatum z-stacked montages were captured using a Nikon Ni-E Eclipse microscope equipped with a DS-Fi1 camera at 20× magnification and processed in the NIS-Elements imaging software (Nikon). Each z-stack tile was captured using a 0.9 μm step size. The montages were then loaded onto Stereolnvestigator and outlines of the striatum were defined. A set of 200×200 μm counting frames was placed by the software in a systematic random fashion within a 400×400 μm grid covering the outlined striatum of each section. Counting was performed in the entire section height (without guard zones) and cells were counted based on their immunolabelling in the optical section in which they first came into focus.

Statistical Analysis and Reproducibility

Samples exhibiting artifacts related to technical issues from experimental procedures—such as mistargeted injections, overt surgical damage, or injections into gliotic foci—were excluded from this study. Statistical tests were performed using GraphPad Prism 9. For comparisons between more than two groups, one-way analysis of variance (Tukey's multiple comparison test) was applied. For comparisons between two groups with more than two factors, two-way analysis of variance (Šidák's multiple comparison test) was applied. When comparing between two matched groups, paired two-tailed t-tests were applied for normally distributed data sets, while for unmatched groups, unpaired two-tailed t-tests were applied. Significance was defined as P<0.05. Respective P values were stated in the figures whenever possible, otherwise, ****P<0.0001, ***P<0.001, **P<0.01, *P<0.05. The number of replicates is indicated in the figure legends, with n denoting the number of independent experiments. Data are represented as the mean±standard error of mean (s.e.m).

Apoptosis Assay

Identification of apoptotic cells within human cell populations was accomplished by terminal deoxynucleotidyl transferase-dUTP nick end labeling (TUNEL) together with immunostaining for their respective fluorescent reporters. TUNEL was performed using the Click-iT TUNEL Alexa Fluor 647 Imaging Assay (Invitrogen, cat. no. C10247) following manufacturer's instructions with the exception that samples were incubated in Proteinase K solution for 20 minutes at room temperature. To confirm efficient TUNEL staining in fixed-frozen brain cryosections, positive control sections were treated with DNase I following manufacturer's instructions. Following TUNEL, sections were immunolabeled for fluorescent reporters following the previously described immunostaining protocol.

Quantification of TUNEL⁺ Human Cells

To assess the distribution and proportion of apoptotic cells within each human cell pool, whole striatal montages of 5 equidistantly spaced, 480 μm apart, sagittal sections spanning the entire striatum were captured using a Nikon Ni-E Eclipse microscope equipped with a DS-Fi3 camera, at 10× magnification and stitched in the NIS-Elements imaging software. The striatum was outlined within each section, and immunolabeled human cells identified and mapped based on their TUNEL labelling within the outlined striatum using Stereo Investigator.

Representative images showing whole humanized striata were generated from previously acquired whole brain montages using the ‘crop’ function and adjusting the ‘min/max’ levels in NIS-Elements imaging software. Representative images of human glial competitive interfaces were then captured as large field z-stacked montages, using a Nikon Ti-E C2+ confocal microscope equipped with 488 nm, 561 nm and 640 nm laser lines, and a standard PMT detector. Images were captured at 40× or 60× magnification with oil-immersion objectives and stitched in NIS-Elements. Maximum intensity projections were then generated, and the ‘min/max’ levels adjusted in NIS-Elements. Similarly, representative images of human cell phenotype were captured, imaged, and processed as z-stacks using the Nikon Ti-E C2+ confocal and the same laser lines.

Fluorescence Activated Cell Sorting (FACS) of Human Glia from Chimeric Mice

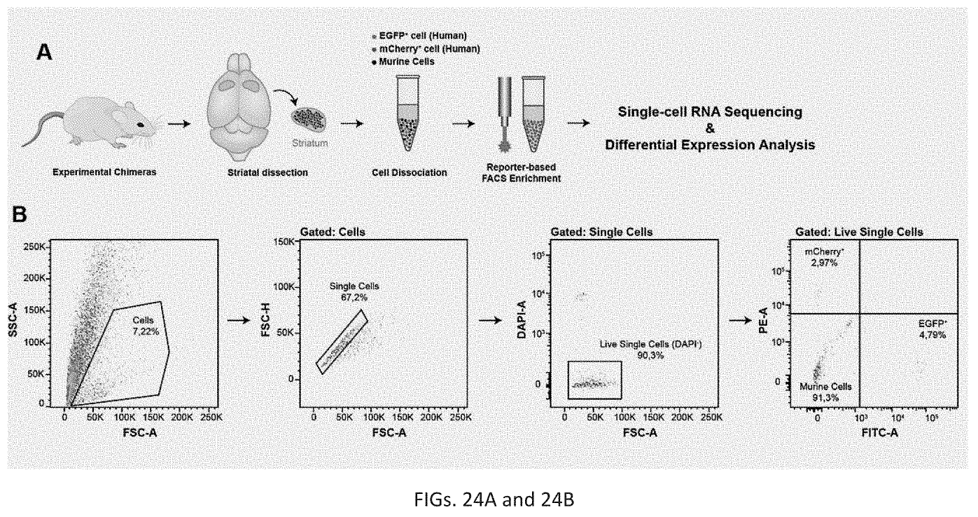

To isolate human cells for scRNA-seq, experimental chimeras were perfused intracardially with HBSS, their striata dissected and tissue dissociated as previously described (Marian, J. N., Zou, L. & Goldman, S. A. in Oligodendrocytes. Methods in Molecular Biology Vol. 1936 (eds David Lyons & Linde Kegel) 311-331 (Humana Press, 2019), and as illustrated in FIG. 24A. Single cell suspensions were isolated based on their expression of mCherry, EGFP, or their absence, using a BD FACSAria Fusion (BD Biosciences). To exclude dead cells, 4′,6-diamidino-2-phenylindole (DAPI; ThermoFisher cat. no. D1306) was added at 1 μg/ml. The gating strategy is shown in FIG. 24B.

Single-Cell RNA Sequencing Analysis

Primary data acquisition Isolated cells were captured for scRNA-seq on a 10× Genomics chromium controller (v3.1 chemistry). Libraries were generated according to manufacturer's instructions and sequenced on an Illumina NovaSeq 6000 at the University of Rochester Genomics Center. scRNA-seq libraries were aligned with STARsolo using a custom two-pass strategy. First, an annotated chimeric GRCh38 and GRCm38 reference was generated using Ensembl 102 human and mouse annotations, with the addition of mCherry and EGFP. STARsolo was then run with parameters: twopassMode=basic, limitSjdbInsertNsj=3000000, and soloUMIfiltering=MultiGeneUMI. BAM files were then split by species, and cross-species multimapping reads were assigned to both human or mouse BAMs. FASTQ files were re-generated from either the mouse or human BAM files and re-aligned to a single species reference.

Differential expression analysis Human data were imported into R using Seurat. Cells were filtered (Unique genes >250 and percent mitochondrial genes <15). Cells were then further filtered for expression of mCherry or EGFP. Counts were imported into Python for integration using scvi where the 4,000 most variable features were used. The model was trained for integration using the mouse sample and cell line in addition to the number of unique genes and percent mitochondrial gene expression. The latent representation was then used for dimensionality reduction via UMAP and Louvain community detection. Smaller populations of cells were classified into six major types of glia based on marker expression. Data were then re-imported into Seurat, and differential expression was carried out using MAST. Genes were considered for differential expression if their expression was detected in at least 3% of all GPCs. The model design for differential expression utilized the number of unique genes in a cell and the experimental group (cell line/age of the cell, and if the cell was in the presence or absence of an opposing clone). Significance for differential expression was P<0.05, with a log 2-fold change of at least 0.15. Ingenuity Pathway Analysis (QIAGEN) was using for functional analysis of each differentially expressed gene list.

Cell cycle analysis G2M scores of each experimental group were calculated using Seurat's CellCycleScoring function. Statistical comparisons between each model's experimental groups were then calculated using Dunn tests with Benjamini-Hochberg multiple comparison adjustments.

Identification of transcription factor-associated regulons Genes were first filtered to retain only those that expressed at least 3 counts in at least 1% of the cells. All 9,579 cells were used in this analysis. The filtered raw matrix was then used as input for the standard pipeline of pySCENIC to identify each transcription factor and its putative downstream targets in the data set. These gene sets are referred to as regulons and are assigned “Area Under the Curve” (AUC) values to represent their activities in each cell, with higher values indicating a stronger enrichment of such regulon. The resulting AUC matrix were then used to look for important transcription factors. Within the GPC subpopulation in both the isograft and allograft models, 1 was assigned to cells from the young WT samples, and 0 to cells from the aged WT or aged HD samples. Lasso logistic regression was then performed on predetermined 0/1 outcome with all TF's AUCs as predictor using glmnet. Lambda for logistic regression was automatically defined with cv.glmnet.

Inventors isolated TFs with positive coefficients, and further filtered based on their mean activity per group, such that TF mean activity in the young WT should be higher than that in the aged counterpart. The final step was to perform gene set enrichment analysis (GSEA) on regulons identified thus far, to determine if they were enriched for differentially upregulated genes in young WT cells compared to aged HD and WT cells (adjusted P<10-2, NES>0).

Identification of co-expressed gene sets with competitive advantage Inventors filtered to exclude genes with fewer than 1 count across all cells, and used the resulting matrix to denoise data with DCA. Weighted gene co-expression network analysis (WGCNA) was performed on denoised data of the GPC subset. A signed network adjacency was calculated with soft thresholding power of 9. Modules were detected after hierarchical clustering of genes on topological overlap matrix-based dissimilarity and dynamic tree cut. Inventors then identified modules whose gene members represented a significant overlap with the important TF targets identified above, using GeneOverlap (adjusted P<10⁻²). The relative contribution of the linearly-independent covariates age (young, aged) and genotype (HD, WT) towards the additive explanation for each module eigengene (e.g., ME˜age+ genotype) was calculated by the 1 mg method, implemented in the relaimpo package.

Network representation: Functional annotation of transcription factors' gene targets was performed with IPA. To create a representative network, inventors focused on the MYC regulon and its shared targets with other important TFs. Networks were constructed with Cytoscape.

Embryoid Body (EB) Generation

To generate uniform EBs, hESCs were dissociated to small clusters with ReLeSR, harvested, and counted with the automated cell counter NucleoCounter NC-200. A total of 3×10⁶ hESCs were added per well of a AGGREWELL-800 plate (StemCell Technologies, cat. no. 34815) and centrifuged to aggregate the hESCs in the individual microwells. Aggregated hESCs were cultured overnight with mTeSR1 supplemented with 10 μm Y-27632 to allow for EB formation. 24h following aggregation, EBs were released from each microwell by gently pipetting medium in each well using a P1000 pipette with a cut tip and transferred into ultra-low attachment tissue culture flasks (Corning, cat. no. 3815) for further directed differentiation. Prior to aggregation, the AGGREWELL-800 plates were prepared according to the manufacturer's guideline.

Flow Cytometry of hESC-Derived Glial Cultures

Glial cultures were collected as a single cell suspension following 5 min dissociation in Accutase, counted with a hemocytometer, and resuspended at 10⁶ cell/ml in MILTENYI wash buffer (MWB; PBS+0.5% BSA Fraction V (ThermoFisher cat. no. 15260037)+2 μM EDTA (ThermoFisher cat. no. 15575020)). Each cell suspension was then incubated in MWB for 15 mins at 4° C. to block non-specific antibody binding and divided in 100 μL fractions for immunolabelling. Each fraction was then incubated with fluorophore-conjugated antibodies for 15 min at 4° C., except for the unstained gating controls. Antibody sources and concentrations are listed in the table below.

| | | Host | | | Catalog |
|---|---|---|---|---|---|
| Type | Antigen | Species | Dilution | Manufacturer | Number |
| Primary | Olig2 | Mouse | 1:200 | MILLIPORE | MABN50 |
| Primary | hGFAP | Mouse | 1:200 | BIOLEGEND | SMI-21 |
| Primary | hN | Mouse | 1:200 | ABCAM | ab254080 |
| Primary | Ki67 | Rabbit | 1:200 | INVITROGEN | MA5-14520 |
| Primary | EGFP | Chicken | 1:500 | INVITROGEN | A10262 |
| Primary | mCherry | Rat | 1:500 | INVITROGEN | M11217 |
| Primary | PDGFRa | Rabbit | 1:200 | CELL | 5241S |
| | | | | SIGNALLING | |
| Primary | Oct-4 | Mouse | 1:100 | MILLIPORE | MAB4401 |
| Secondary | Rat IgG (H + L) - | Goat | 1:400 | INVITROGEN | A-11077 |
| | Alexa Flour 568 | | | | |
| Secondary | Chicken IgY (H + L) - | Goat | 1:400 | INVITROGEN | A32931 |
| | Alexa Flour Plus 488 | | | | |
| Secondary | Rabbit IgG (H + L) - | Goat | 1:400 | INVITROGEN | A32733 |
| | Alexa Fluor Plus 647 | | | | |
| Secondary | Mouse IgG (H + L) - | Goat | 1:400 | INVITROGEN | A32728 |
| | Alexa Fluor Plus 647 | | | | |
| Conjugated | CD140a-FITC | Mouse | 1:10 | BD HORIZON | 564594 |
| Conjugated | CD140a-PE | Mouse | 1:10 | BD PHARMINGEN | 556002 |
| Conjugated | CD44-APC | Mouse | 1:500 | MILTENYI | 130-113-331 |
| | | | | BIOTEC | |
| Conjugated | A2B5-APC | Mouse | 1:50 | MILTENYI | 130-093-582 |
| | | | | BIOTEC | |

Cells were then washed with MWB, spun for 10 min at 200×g, resuspended in MWB, and strained into 5 ml polystyrene tubes with 35 μm cell-strainer caps (Corning, cat. no. 352235). To exclude dead cells, 4′,6-diamidino-2-phenylindole (DAPI; ThermoFisher cat. no. D1306) was added at 1 μg/mL. Flow cytometry analysis of glial cultures was then performed on a CYTOFLEX S platform (Beckman Coulter), and the data analyzed with the CYTEXPERT (Beckman Coulter) and FLOWJO (BD Biosciences) software. Gating strategy and data analysis are exemplified in FIG. 18.

Immunocytochemistry of hESC and Glial Cultures

Cultures were fixed with 4% paraformaldehyde (PFA) for 7 mins, washed with phosphate-buffered saline (PBS) and then permeabilized and blocked with permeabilization/block buffer (PBS+0.1% Triton-X (Sigma-Aldrich cat. no. T8787)+1% BSA Fraction V) for 1h. Cultures were then incubated overnight with primary antibodies at 4° C., washed with PBS, and then incubated with secondary antibodies at room temperature for 2h. Antibody sources and concentrations are listed in the table above. Nuclear counterstain was then performed by incubating with 1 μg/mL DAPI for 5 mins at room temperature, and then washed with PBS an additional 3 times prior to imaging.

Representative images of hESCs were acquired on a Nikon Eclipse Ti microscope equipped with a DS-Fi3 camera at 10× magnification while representative images of glial cultures were captured with a DS-Qi2 camera at 20× magnification, and ‘min/max’ levels were adjusted for both in NIS-Elements imaging software (Nikon).

Mapped Cell Counting and Section Volume Estimation (Volumetric Quantification)

As previously mentioned in the methods section, mapped cells are counted as 1 if their respective representative line segments are fully inside, 0 if they are fully outside, and partially if they are intersecting the radiating spherical shell. To that end, inventors calculate the points on the surface of the radiating spherical shells corresponding to the projection of mapped cells onto the spherical shell. Inventors here consider only the calculation of points above the injection site since points below are similarly handled. The corresponding points on the spherical shell are given by:

z_{d}(r)=\sqrt{r²−d_{xy}²}

where r is either a or b, depending on the if the shell intersects the point at the outer or inner surface. If zₗ>z_{d}(b) the point is outside the shell and thus not counted, if zᵤ<z_{d} (a), the shell has passed beyond the point and it is not counted. If z_{d}(a)<zᵤ and z_{d}(b)>zₗ, the line segment is completely within the spherical shell and it is counted as 1. Additionally, inventors may have the two limiting cases where the spherical shell intersects the line segment. These two examples are similar, so inventors deal only with the case where the line segment intersects the upper surface of the spherical shell. That is, the case where z_{d}(a)>zᵤ and zᵤ<z_{d} (b) <zₗ. In this case, the part of the line segment inside the spherical shell has length zᵤ−z_{d}(b) and the mapped cell was counted by integrating its point probability function as:

(zᵤ−z_{d}(b))/w

where w is the width of the section.

To calculate the corresponding section volume within which mapped cells were counted, inventors first triangulate each polygon representing the anatomical boundary of each section. Inventors then form a prism from the triangles with height matching the section thickness. Inventors represented each prism as 3 tetrahedra and measure to cumulative volume inside the sphere as the total overlap volume between the sphere of radius r and each tetrahedra.

For a section with depth coordinate zₗ, each triangle is represented by 3 points v₁, v₂, v₃ with coordinates (x₁, y₁, zₗ), (x₂, z₂, zₗ), and (x₃, y₃, zₗ). These triangles together make a 2D representation of the domain. To get a 3D representation of each section, inventors used the known thickness of dz (here 20 nm) and thicken the triangle into a prism shape with 3 new points w₁, w₂, w₃ translated perpendicular the section plane by dz upon the upper boundary of the section at coordinates (x₁, y₁, zᵤ), (x₂, y₂, zᵤ) and (x₃, y₃, zᵤ). Calculating the exact overlap volume of a 3D polygon and a sphere is not trivial, but inventors can calculate the overlap volume of spheres and tetrahedra. To that end, inventors covered each prism domain by 3 tetrahedra given by the coordinate sets [symbol] (v₁, v₂, v₃, w₁ [symbol] , [symbol] v₁, v₃, w₁, w₂ [symbol] , and [symbol] v₃, w₁, w₂, w₃ [symbol] . Given a sphere of radius r, the intersecting volume of each section with the sphere is then given by the sum over the volume of the intersection of a sphere S of radius r and each tetrahedra T:

$$V ( r ) = {\sum }_{T} \vert {S}_{r} ⋂ T \vert$$

### Example A1—Generation of Distinctly Color-Tagged Human Glia from WT and HD hESCs

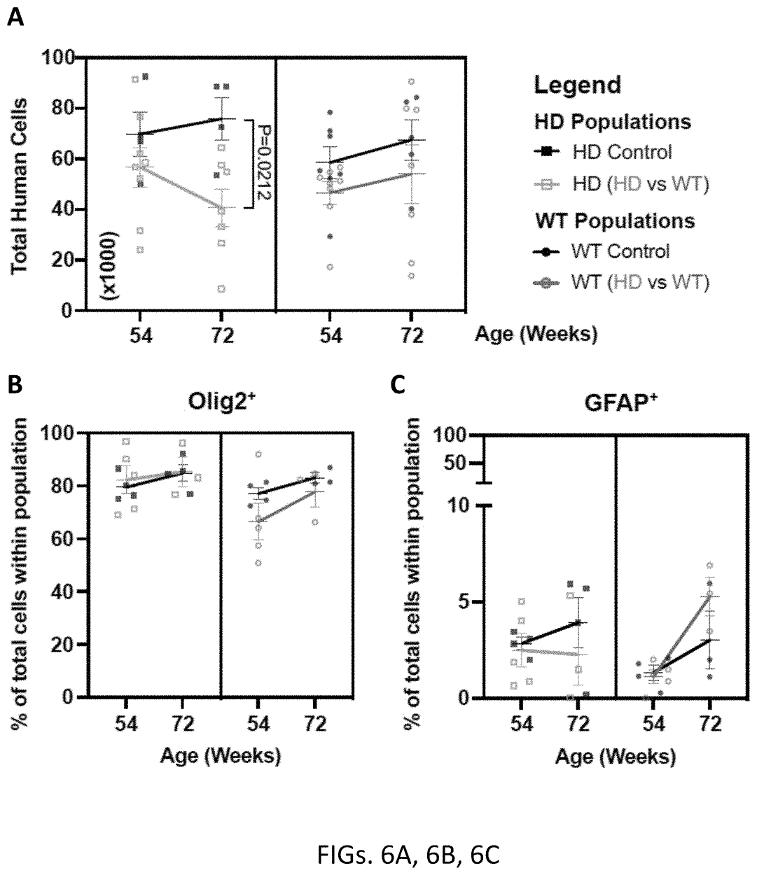

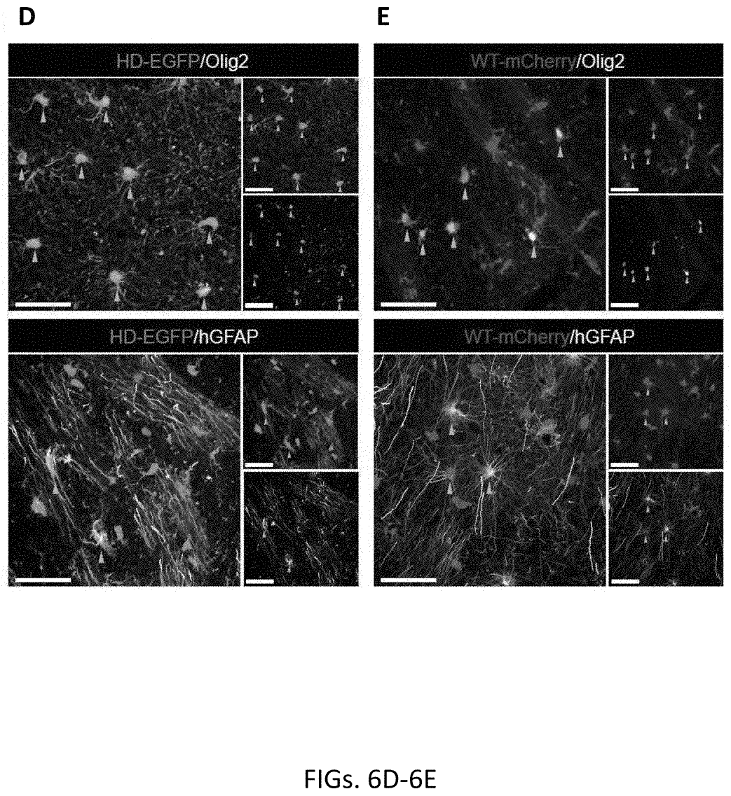

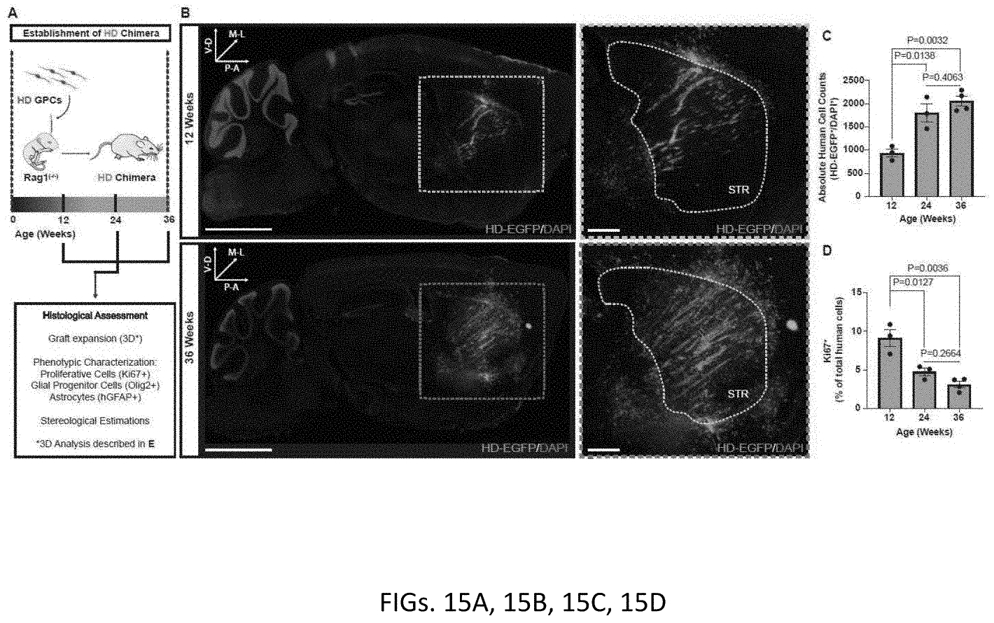

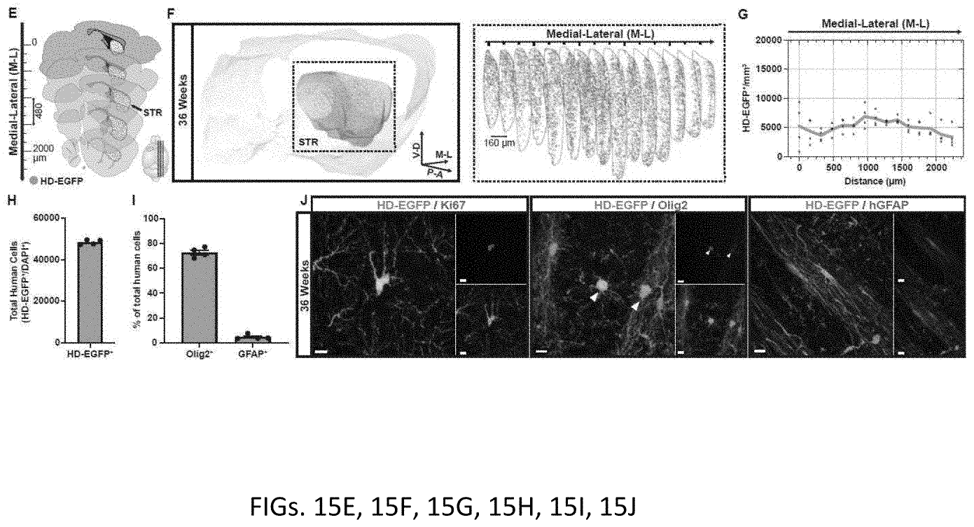

To assess the ability of healthy glia to replace their diseased counterparts in vivo, inventors first generated fluorophore-tagged reporter lines of WT and HD human embryonic stem cells (hESC), so as to enable the production of spectrally-distinct GPCs of each genotype, whose growth in vivo could then be independently monitored. A CRISPR-Cas9-mediated knock-in strategy (Oceguera-Yanez, F. et al. Methods 101, 43-55 (2016)) was first used to integrate EGFP and mCherry reporter cassettes into the AAVS1 locus of matched, female sibling wild-type (WT, GENEA019) and mHtt-expressing (HD, GENEA020) hESCs (Dumevska, B. et al. Stem Cell Research 16, 430-433 (2016) and Dumevska, B. et al. Stem Cell Research 16, 397-400 (2016)) (FIG. 1). Inventors then verified that the reporter cassettes stably integrated into each of these clones (FIG. 1D), and that editing did not influence the self-renewal, pluripotency, or karyotypic stability of the tagged hESCs (FIGS. 1E and 2A). From these tagged and spectrally-distinct lines, inventors used a previously described differentiation protocol (Benraiss, A. et al. Nature Communications 7, 11758 (2016)) to produce color-coded human glial progenitor cells (hGPCs) from each line, whose behaviors in vivo could be compared, both alone and in competition. Inventors validated the ability of each line to maintain EGFP or mCherry expression after maturation as astrocytes or oligodendrocytes, and their lack of any significant differentially-expressed oncogenic mutations, or copy number variants (CNVs) that could bias growth (FIGS. 2B and 2C); inventors also verified that both the WT and mHTT-expressing hGPCs, when injected alone, colonized the murine host brains (FIGS. 15 and 6).

Inventors then differentiated both WT-mCherry and HD-EGFP hESCs using a established protocol for generating hGPCs (Wang, S. et al. Cell Stem Cell 12, 252-264 (2013)) and assessed both their capacity to differentiate into glia and the stability of their reporter expression upon acquisition of glial fate (FIG. 3). By 150 days in vitro (DIV), glial cultures derived from both WT-mCherry and HD-EGFP were equally enriched for PDGFRα⁺/CD44⁺ bipotential GPCs (P=0.78), comprising around half of the cells in the cultures, with the rest being immature A2B5⁺ GPCs and PDGFRα⁻/CD44⁺ astrocytes and their progenitors (FIG. 3 and FIG. 18). Importantly, virtually all immune-phenotyped cells derived from WT-mCherry and HD-EGFP hESCs—including mature astrocytes as well as GPCs—continued to express their respective fluorescent reporter, indicating that transgene expression remained stable upon acquisition of terminal glial identity (FIG. 3D).

### Example A2—Establishment of Human HD Glial Chimeric Mice

Murine chimeras with striata substantially humanized by HD glia (HD chimeras, FIG. 15) were generated to provide an in vivo model by which to assess the replacement of diseased human glia by their healthy counterparts. hGPCs derived from mHtt-expressing hESCs engineered to express EGFP (FIG. 1, FIG. 2, and FIG. 3; henceforth designated as HD) were implanted into the neostriatum of immunocompromised Rag1^{(-/-)} mice and monitored their expansion histologically (FIG. 15A).

Following implantation, HD glia rapidly infiltrated the murine striatum, migrating and expanding firstly within the striatal white matter tracts (FIG. 15B). Gradually, these cells expanded outwards, progressively displacing their murine counterparts from the striatal neuropil, so that by 36 weeks, the murine striatum was substantially humanized by HD glia (FIGS. 15B, 15F, and 15G). The advance of HD glia was driven by their mitotic expansion, with their total number doubling between 12 and 36 weeks (FIG. 15C; P=0.0032). Inversely, as they expanded and matured within their newly established domains, their proliferative cell pool (Ki67⁺) was progressively depleted (FIG. 15D, and I; P=0.0036), slowing their expansion rate over time.

Most of the HD glia expanded as Olig2⁺ GPCs (72.7±1.9%), which persisted as the new resident pool after replacing their murine counterparts. A fraction of these (4.8±0.9%) further differentiated into GFAP⁺ astrocytes (FIGS. 151 and 15J). Astrocytic differentiation was mostly observed within striatal white matter tracts. These sick astrocytes lacked the structural complexity typically observed in healthy counterparts and displayed abnormal fiber architecture, as previously reported (FIG. 15J; Osipovitch, M. et al., “Human ESC-Derived Chimeric Mouse Models of Huntington's Disease Reveal Cell-Intrinsic Defects in Glial Progenitor Cell Differentiation,” Cell Stem Cell 24: 107-122 (2019), which is hereby incorporated by reference in its entirety).

### Example A3—Healthy WT hGPCs Infiltrate the HD Chimeric Adult Striatum and Outcompete Resident Glia

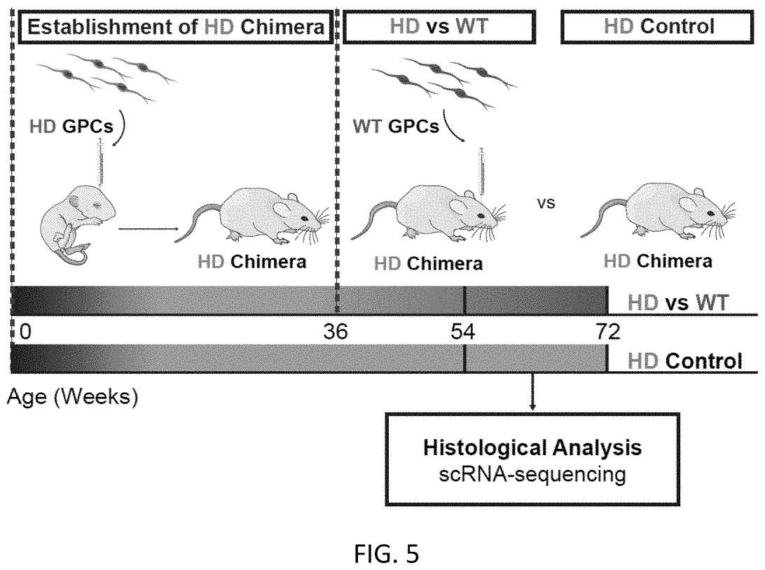

The established chimeras whose striatal glia are largely mHTT-expressing and human were used to determine how the resident HD human glia might respond to the introduction of healthy hGPCs and whether the resident glial populations might to some extent be replaced. hGPCs derived from WT hESCs engineered to express mCherry (FIG. 1, FIG. 2, and FIG. 3; henceforth designated as WT) were engrafted into the striatum of 36 weeks old HD chimeras and monitored for expansion using histology as they competed for striatal domination (FIG. 5).

Following engraftment, WT glia pervaded the previously humanized striatum, gradually displacing their HD counterparts as they expanded from their implantation site (FIG. 4). This process was slow but sustained, over time yielding substantial repopulation of the HD striatum (FIG. 4; 54 weeks, p<0.0001; 72 weeks, p<0.0001). Remarkably, the expansion of WT glia was paralleled by a concurrent elimination of HD glia from the tissue (as opposed to their spatial relocation) (FIG. 4; 54 weeks−P<0.0001, 72 weeks−P<0.0001), and was typically characterized by a discrete advancing front behind which almost no HD glia could be found (FIG. 4).

Mutually exclusive domains formed in the wake of competition between Olig2⁺ GPCs (FIG. 4). These comprised most of the WT glial population (80.1±4.7% at 72 weeks), which persisted as the new resident GPC pool after replacing their HD counterparts. Their potential to generate astroglia was maintained, as a fraction of these (4.0±1.5% at 72 weeks) further differentiated into GFAP⁺ astrocytes (FIG. 6) within their newly established domains. Curiously, within regions dominated by WT glia, HD astrocytes (GFAP⁺) lingered, primarily within white matter tracts (FIG. 4). Nonetheless, the overall ratio between Olig2⁺ and GFAP⁺ glia remained stable throughout the experiment in both populations (FIG. 6) indicating that while GPC replacement precedes astrocytic replacement, proportional phenotypic repopulation is achieved over time.

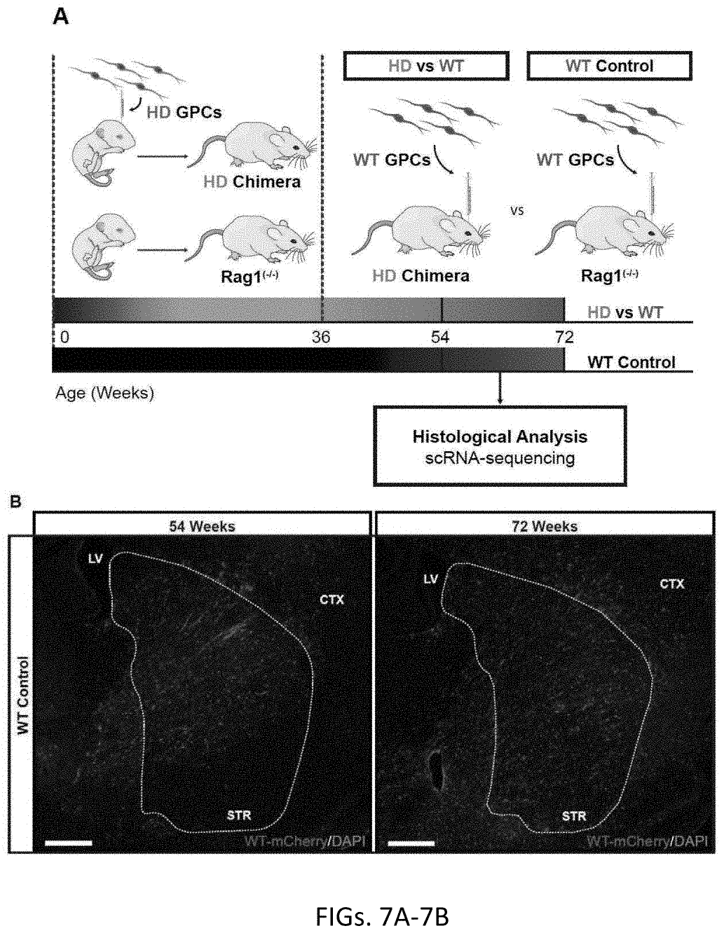

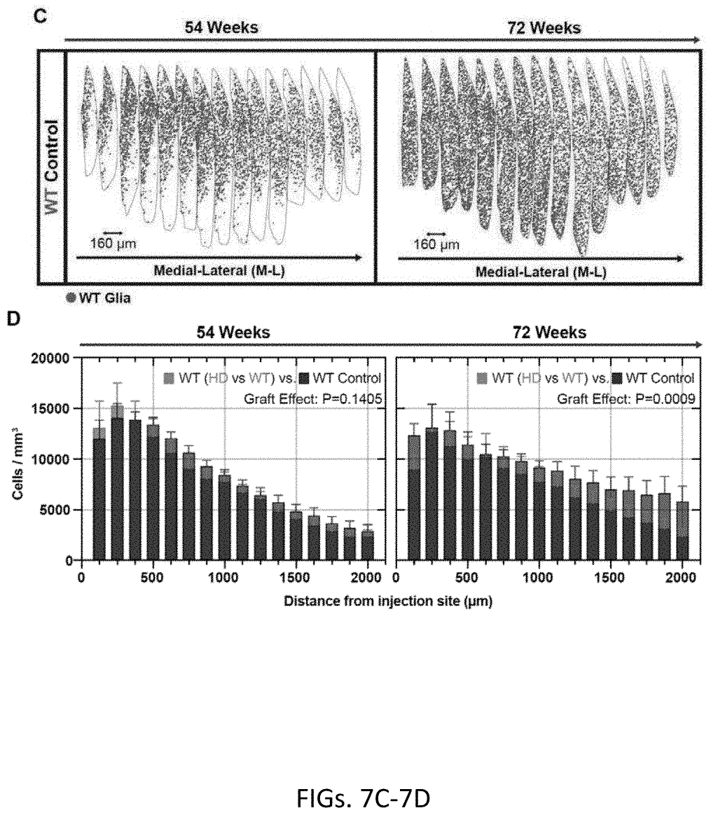

Interestingly, human-human glial replacement developed at a slower rate than human-murine glial replacement, as WT hGPCs implanted into naïve adult Rag1^{(-/-)} mice expanded throughout the host striatum more broadly than those grafted into neonatally-chimerized adult Rag1^{(-/-)} mice (FIG. 7; 54 weeks: P=0.14, 72 weeks: P=0.0009). These results indicate that competitive glial replacement develops with species-specific kinetics that differ between xenogeneic and allogeneic grafts.

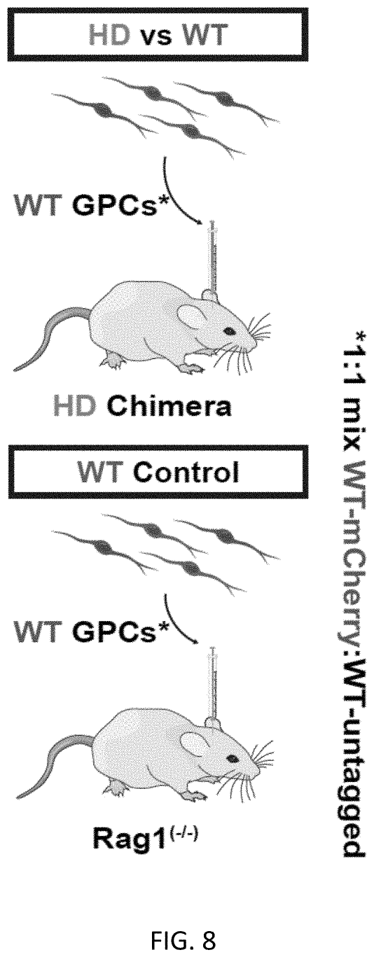

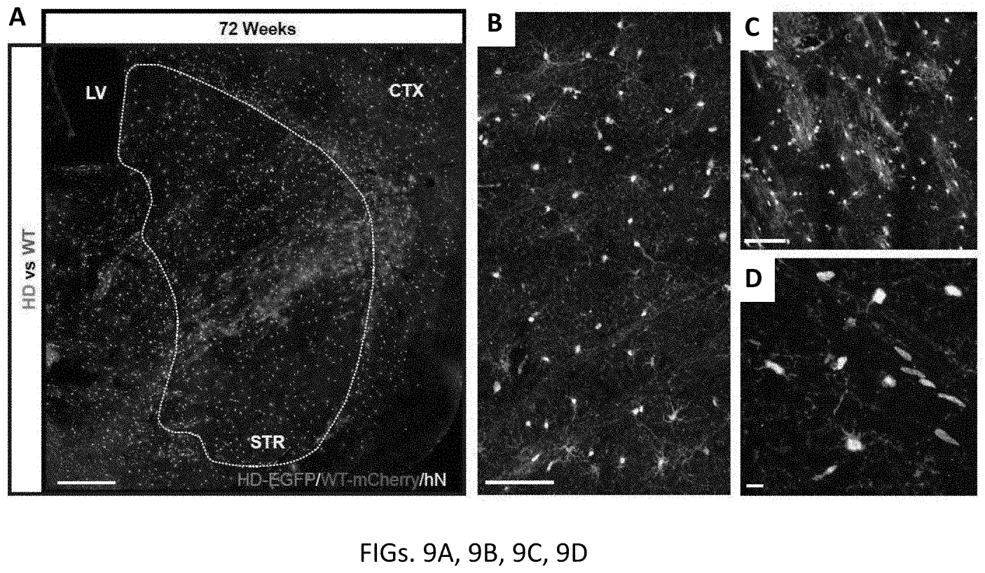

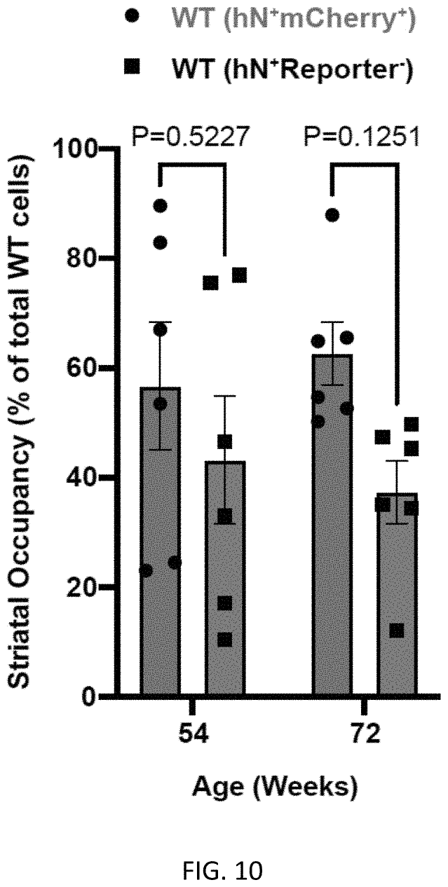

These results were not an artifact of off-target effects derived from gene editing nor fluorescent reporter expression toxicity, as co-engrafted hGPCs derived from WT-mCherry and their unmodified counterparts (WT-untagged) (FIG. 8), expanded equally within the striatum of HD chimeras and yielded analogous glial repopulation (FIG. 9 and FIG. 10; 54 Weeks—P=0.5227-72 Weeks—P=0.1251). As such, analysis done in (FIG. 4) and (FIG. 6 and FIG. 7) reports samples from both experimental paradigms. Remarkably, while WT and HD glia strongly segregated from each other, the two isogenic clones of WT glia could be found admixing (FIG. 9), indicating that active recognition precedes competitive elimination of HD glia from the tissue.

### Example A4—Human WT Glia Enjoy a Proliferative Advantage Relative to Resident HD Glia

Striatal humanization by HD glia progressed with a gradual exhaustion of their proliferative cell pool as they expanded and matured within the tissue (FIG. 15D). Therefore, whether the selective expansion of younger WT glia within the HD striatum was sustained by a difference in proliferative capacity between the two populations was tested. The temporal expression of Ki67 in both WT and HD glial populations was assessed as competitive striatal repopulation unfolded.

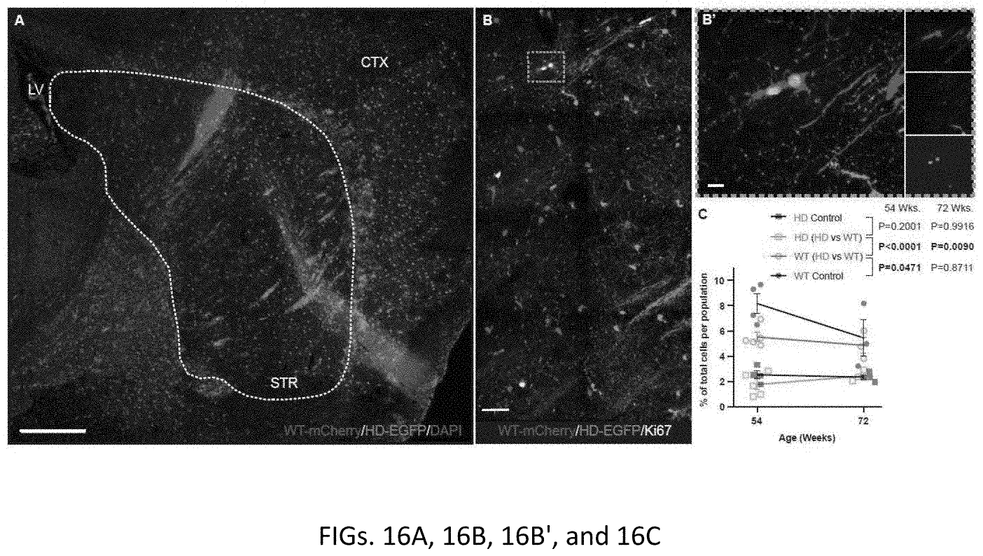

At both 54 and 72 weeks of age, the mitotic fraction of implanted WT glia was significantly larger than that of resident HD glia (FIGS. 16A-C; 54 weeks—P<0.0001, 72 weeks—P=0.0120). These data indicate that the repopulation of the HD striatum by WT glia was fueled by a relatively enriched proliferative cell pool. It's important to note that while this proliferative advantage became less pronounced as the cells aged, it was maintained throughout the experiment. With this in mind, the sustained proliferative advantage of implanted WT glia over their HD counterparts, should provide a driving force for continuous striatal repopulation beyond the observed experimental timepoints (FIGS. 16A-16C).

### Discussion of Examples A1-A4

The striata of human glial chimeric mice harboring HD-derived glia are repopulated by their healthy counterparts, following implantation of WT human GPCs (FIG. 4). The data presented supra suggest that this process was driven by a recapitulation of developmental cell-cell competition (Amoyel, M. & Bach, E. A., Development 141: 988-1000 (2014) and Baker, N. E., Nat Rev Genet 21,683-697 (2020), which are hereby incorporated by reference in their entirety), dynamically effected in the adult brain. Such cell-cell competition has traditionally been defined by the active purging of relatively slowly growing cells from the tissue, upon their interaction with faster growing neighbors (Morata, G. & Ripoll, P., Dev Biol 42: 211-221 (1975), Simpson, P., Dev Biol 69: 182-193 (1979), and Simpson, P. & Morata, G., Dev Biol 85,299-308 (1981), which are hereby incorporated by reference in their entirety). Accordingly, WT human GPCs typically expanded from their implantation sites in advancing waves that, upon contact, repulsed and eliminated their hitherto stably resident HD-derived counterparts (FIG. 4). The expansion of WT hGPCs in this HD glial environment was propelled by a sustained proliferative advantage on the part of these young, healthy GPCs, which over time yielded their extensive colonization of the host brain.

The enrichment for MYC targets in their transcriptional signature—one of the most well-described regulators of cell competition (Cova, C. de la, et al., Cell 117: 107-116 (2004), Moreno, E. & Basler, K. Cell 117: 117-129 (2004), and Villa del Campo, C., et al., Cell Reports 8: 1741-1751 (2014), which are hereby incorporated by reference in their entirety)—further indicated the conservation of this mechanism in the elimination of HD hGPCs. In particular, their loss paralleled a depletion of ribosomal transcripts, matching the transcriptional profile of putative ‘loser’ cells during cell competition in the developing mouse embryo (Lima, A. et al. Nat Metabolism 1-18 (2021), which is hereby incorporated by reference in its entirety) and skin (Ellis, S. J. et al. Nature 569: 497-502 (2019), which is hereby incorporated by reference in its entirety). This observation suggests that the regulation of translation and protein synthesis is a determinant of cell competition not only during development, but also in the adult brain.

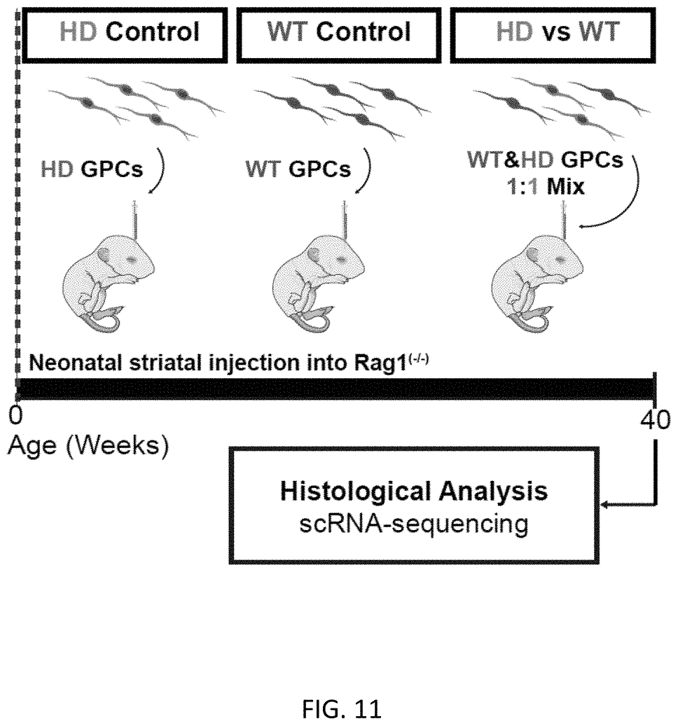

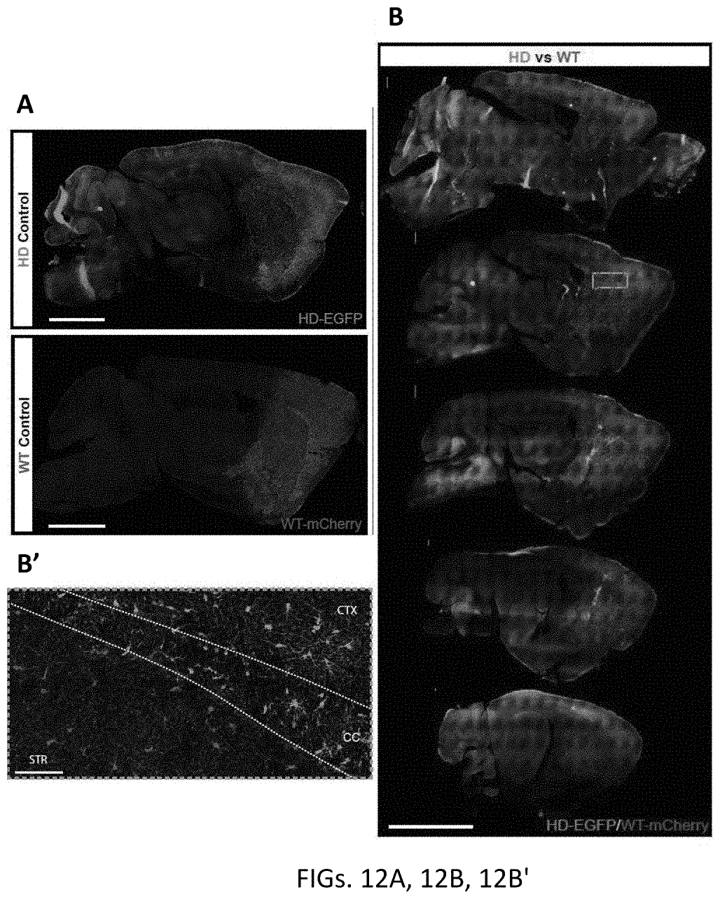

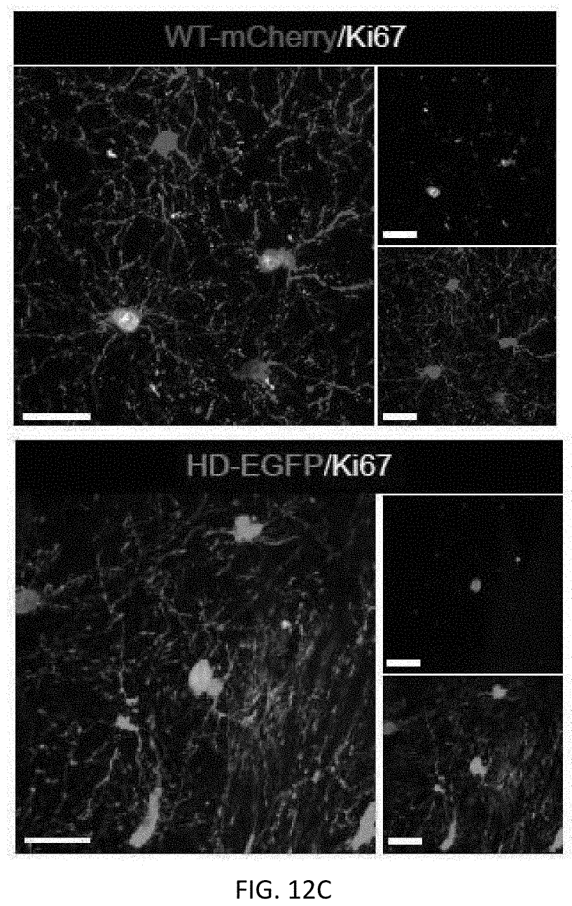

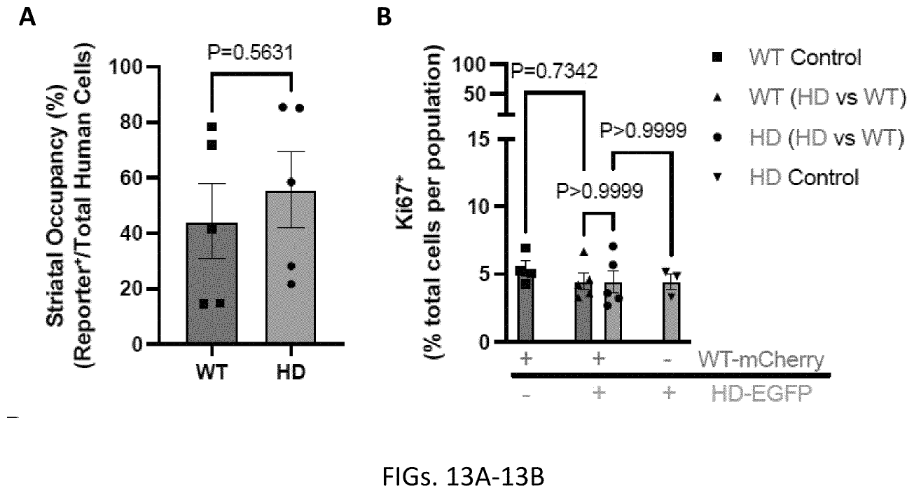

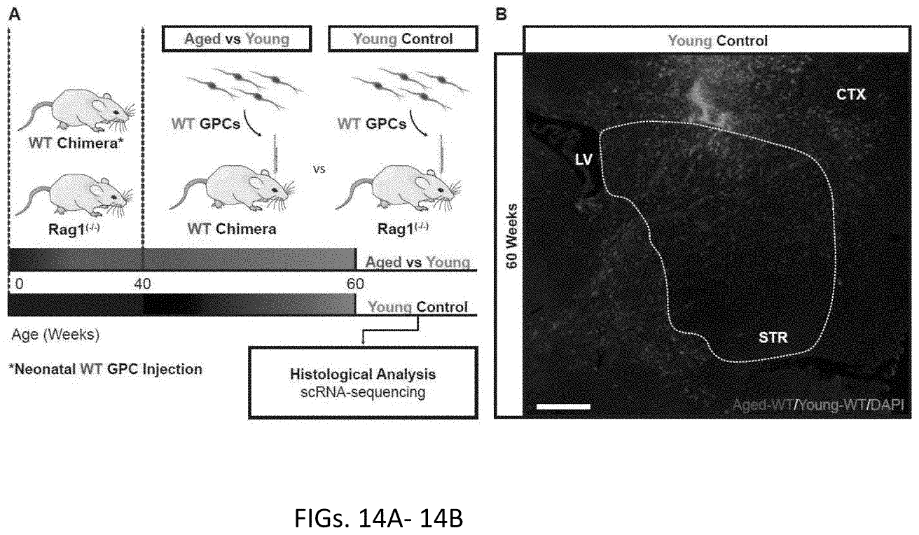

Interestingly, this process cannot be completely explained by the deleterious effects of mHtt-expression, since when co-engrafted, both clones contributed to the humanization of the mouse striatum at the time points assessed (FIG. 11, FIG. 12, FIG. 13). The repopulation of HD-chimeras was rather driven by the age difference between the newly implanted, more proliferative, WT GPCs and the more mature, relatively quiescent, resident HD glia. This notion parallels observations from liver repopulation studies, in which allogeneic engraftment of mouse fetal liver progenitors into older, but otherwise healthy, hosts are associated with faster and more extensive tissue replacement than their engraftment into younger hosts (Menthena, A. et al. Gastroenterology 140: 1009-1020.e8 (2011), which is hereby incorporated by reference in its entirety).

As both the severity and age of onset of HD are closely correlated with CAG repeat length, employing cell lines harbouring mHtt with more CAG repeats might better model its' influence within the short lifespan of these chimeras. Regardless, the competitive advantage imparted by the difference in age—and thus difference in mitotic competence—between resident and newly implanted GPCs, seems a significant contributor to the replacement of the resident pool.

The competitive replacement described here resembles that of murine glial replacement by implanted hGPCs, as their expansion within the murine brain is also sustained by a relative proliferative advantage, and progresses with the elimination of their murine counterparts upon contact. Moreover, this competitive behaviour seems to largely mimic development, where successive waves of GPCs compete amongst each other, with the oldest being almost completely eradicated from the brain by birth and replaced by their younger successors. These commonalities suggest that cell-cell competition may reveal intrinsic developmental programs that can be re-initiated in the adult brain environment following the introduction of new and younger GPCs.

In broad terms, inventors' observations suggest that the brain may be a far more dynamic structural environment than previously recognized, with cell-cell competition among glial progenitor cells and their derived astrocytes as critical in adult brain maintenance as in development. One may readily envision that somatic mutation among glia and their progenitors may yield selective clonal advantage to one daughter lineage or the other, resulting in the inexorable replacement of the population by descendants of the dominant daughter. Such a mechanism may contribute to the accelerated disease progression of disorders in which genomic instability and somatic mutation may yield cells of distinct competitive advantages, which might then have competitive advantage over their sibling clones. This scenario, while typifying the onset of carcinogenesis broadly and gliomagenesis in the brain, may be similarly involved in the development of non-neoplastic adult-onset disorders in which glial cells are causally-involved, such as some childhood onset schizophrenias, and HD itself.

This work lays the foundation for the exploitation, as well as the study, of mechanisms underlying cell-cell competitive interactions between human glia in vivo in a variety of contexts. In practical terms, the present data suggest that diseased human glia may be replaced following the introduction of younger and healthier hGPCs. Indeed, such glial replacement may offer a viable strategy towards the cell-based treatment of a variety of neurological diseases. This study demonstrates that human glia afflicted by a prototypic neurodegenerative disease may be replaced in vivo by healthy counterparts following the implantation of healthy human GPCs. A novel humanized platform was established that allows one to predict the likely efficiency of human glial replacement in a variety of disease contexts, while simultaneously interrogating the mechanisms by which replacement occurs. The mechanistic insights yielded in this study may enable strategies by which to further enhance the speed and extent of human glial replacement following hGPC delivery. Together, these data highlight the potential of hGPCs as a cellular vectors for the treatment of those diseases of the human CNS in which glial cells are causally involved.

### Example A5—Human WT Glia Assume a Dominant Competitor Profile when Encountering HD Glia

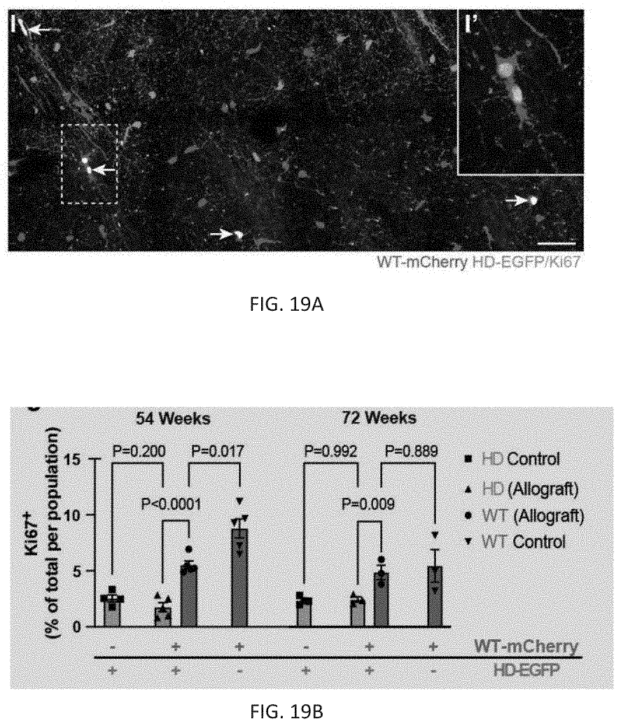

Having established that implanted WT hGPCs effectively colonize the HD glial chimeric striata at the expense of the resident mHTT-expressing glia, inventors next sought to define the molecular signals underlying their competitive dominance. To that end, inventors analyzed the transcriptional profiles of WT and HD human glia isolated from the striata of chimeras in which the two cell populations were co-resident and competing, as well as from their respective controls in which one or the other was transplanted without the other, using single cell RNA-sequencing (scRNA-seq; 10× Genomics, v3.1 chemistry) (FIG. 20A). Following integration of all captures and aligning against human sequence, Louvain community detection revealed six major populations of human glia; these included hGPCs, cycling hGPCs, immature oligodendrocytes (iOL), neural progenitor cells (NPCs), astrocytes, and their intermediate progenitors (astrocyte progenitor cells, APCs) (FIGS. 20B-D). Within these populations, cell cycle analysis predicted a higher fraction of actively proliferating G2/M phase cells in competing WT cells compared to their HD counterparts (FIG. 20E), aligning with the histological observations (FIG. 19). To proceed, inventors focused on hGPCs as the primary competing population in inventors' model. Pairwise differential expression revealed discrete sets of differentially expressed genes across groups (FIG. 20F), and subsequent functional analysis with Ingenuity pathway analysis (IPA) within the hGPC population revealed numerous salient terms pertaining to their competition (FIG. 20G).

It was found that during competition, WT GPCs activate pathways driving protein synthesis, whereas HD GPCs were predicted to downregulate them. Predicted upstream transcription factor activation identified YAP1, MYC, and MYCN—conserved master regulators of cell growth and proliferation—as significantly modulated across experimental groups. Importantly, inventors found YAP1 and MYC targets to be selectively down-regulated in competing HD GPCs relatively to their controls (FIG. 20G). Notably, this down-regulation was attended by a marked repression of ribosomal encoding genes (FIG. 20I). Conversely, competing WT hGPCs showed an upregulation of both YAP1 and MYC targets, as well as in the expression of ribosomal encoding genes, relative to controls (FIGS. 20G-H). As such, these data suggest that the implanted WT hGPCs actively assumed a competitively dominant phenotype upon contact with their HD counterparts, to drive the latter's local elimination while promoting their own expansion and colonization.

### Example A6—Age Differences Drive Competitive Human Glial Repopulation

Since WT cells transplanted into adult hosts were fundamentally younger than the resident host cells that they displaced and replaced, inventors next asked if differences in cell age, besides disease status, might have contributed to the competitive success of the late donor cells. To that end, inventors engrafted hGPCs newly produced from WT hESCs engineered to express EGFP into the striata of 40 week-old adult glial chimeras, which had been perinatally engrafted with hGPCs derived from mCherry-tagged, otherwise isogenic WT hESCs (FIG. 17A). Inventors then monitored the expansion of the transplanted cells histologically, so as to map the relative fitness and competitive performance of these isogenic, but otherwise distinctly aged pools of hGPCs.

We noted that the expansion of implanted WT glia within the striatum of WT chimeras was strikingly similar to their expansion in the striata of HD chimeras (FIG. 4). Following engraftment, the younger WT glia rapidly infiltrated the previously humanized striatum, progressively displacing their aged counterparts as they expanded from their implantation site, ultimately yielding substantial recolonization of the tissue (FIGS. 17B-D and E; P<0.0001). Their expansion was paralleled by the local elimination of aged WT glia (FIGS. 17B-D and F; P<0.0001), which was also marked by a discrete advancing front, behind which few already-resident WT glia could be found (FIG. 17C). Accordingly, inventors also noted that the mitotic fraction of implanted WT glia was significantly larger than that of their resident aged counterparts (FIGS. 17G-I; P=0.018). Together, these data indicated that the repopulation of the human WT glial chimeric striatum by younger isogenic hGPCs was attended by the replacement of the older cells by their younger counterparts, fueled in part by the relative expansion of the younger, more mitotically active cell population.

### Example A7—Young Cells Replace their Older Counterparts Via the Induction of Apoptosis

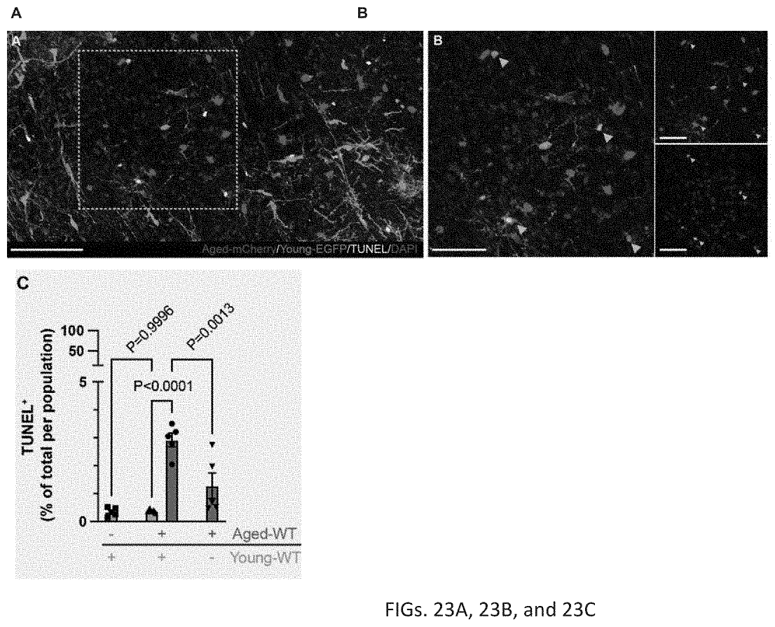

Since younger glia appeared to exert clear competitive dominance over their older counterparts, inventors next asked whether the elimination of the older glia by younger cells occurred passively, as a result of the higher proliferation rate of the younger cells leading to the relative attrition of the older residents during normal turnover, or whether replacement was actively driven by the induction of programmed cell death in the older cells by the more fit younger cells. To address this question, inventors used the TUNEL assay to compare the rates of apoptosis in aged and young WT glial populations as they competed in the host striatum, as well as at their respective baselines in singly-transplanted controls. It was found that as competitive repopulation unfolded, that aged WT glia underwent apoptosis at a markedly higher rate than their younger counterparts (FIGS. 23A-C; P<0.0001). Critically, the increased apoptosis of older, resident glia appeared to be driven by their interaction with younger cells, since a significantly higher proportion of aged glia was found to be apoptotic in chimeras transplanted as adults with younger cells, than in controls that did not receive the later adult injection (FIGS. 23A-C; P=0.0013). These data suggest that aged resident glia confronted by their younger counterparts are actively eliminated, at least in part via apoptosis triggered by their encounter with the younger hGPCs, whose greater relative fitness permitted their repopulation of the chimeric host striatum.

### Example A8—Young hGPCs Acquire a Signature of Dominance when Challenged with Older Isogenic Cells

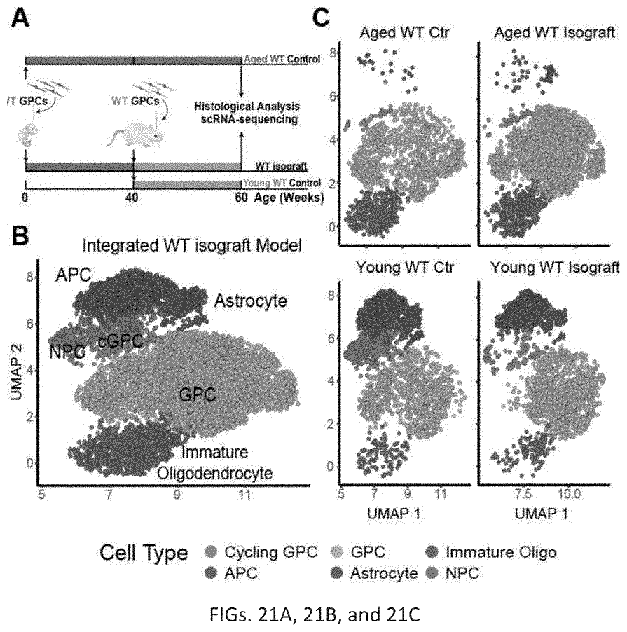

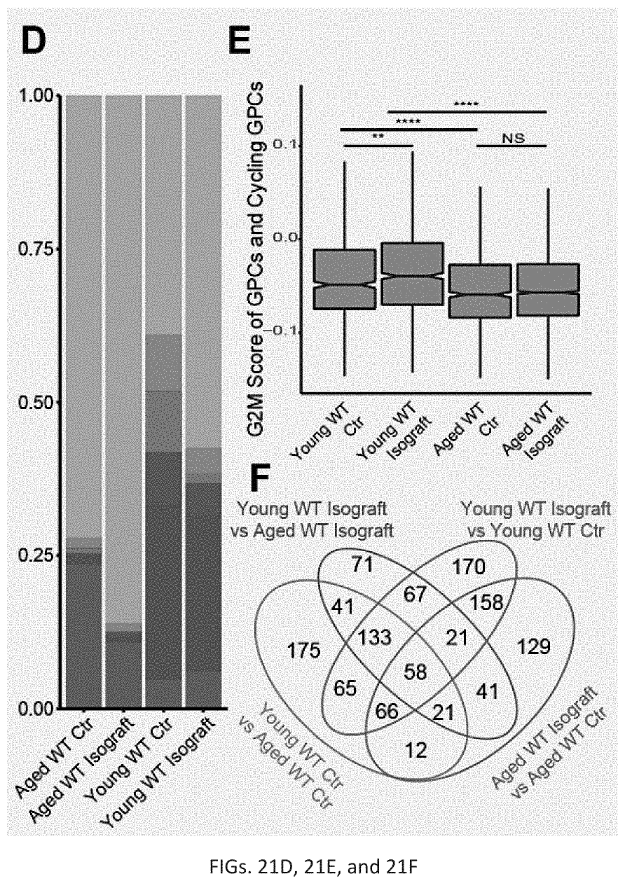

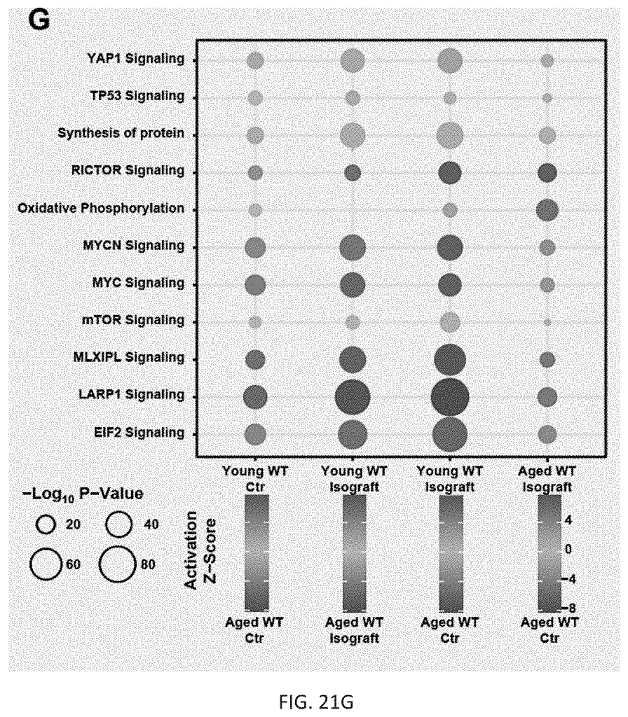

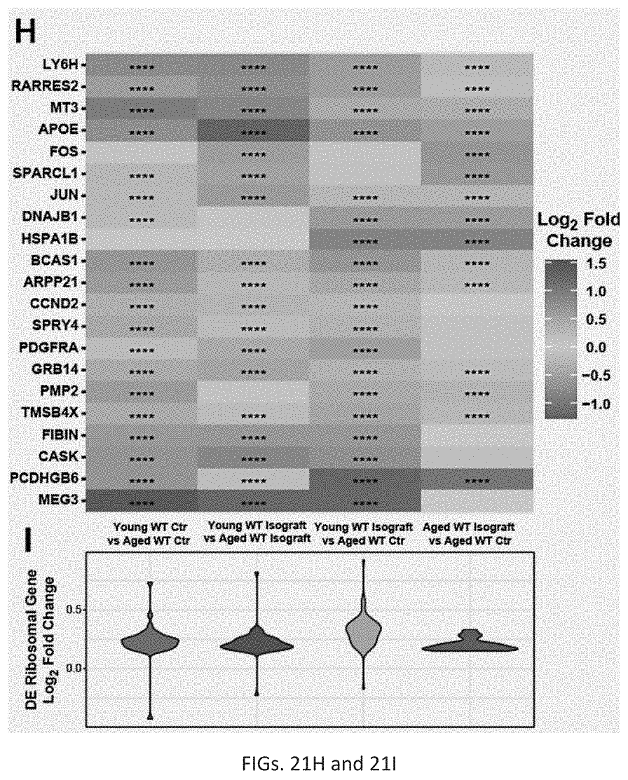

To ascertain if the molecular signals underlying the competitive dominance of younger WT glia over aged WT glia are similar to those underlying their dominance over HD glia, inventors analyzed the transcriptional signatures of competing young and aged WT glia and their respective controls, using scRNA-seq (FIG. 21A). Within the sequenced populations (FIGS. 21B-D), it was noted that the fraction of competing aged WT cells in the G2/M phase of the cell cycle to be markedly lower than their younger counterparts (FIG. 21E), in accord with the histological data (FIG. 17I). Differential expression analysis revealed discrete sets of genes differentially expressed between competing young and aged WT GPCs (FIGS. 21F and H), and subsequent IPA analysis of those gene sets revealed a signature similar to that observed between donor (young) WT and already-resident (aged) HD GPCs in the competitive allograft model (FIG. 21G). In particular, genes functionally associated with protein synthesis, including ribosomal genes as well as upstream YAP1, MYC and MYCN signaling, were all activated in competing young WT GPCs relative to their aged counterparts (FIG. 21G). Yet despite these similarities, in other respects aged WT GPCs responded differently than did HD GPCs to newly implanted WT GPCs. In contrast to HD GPCs, aged WT cells confronted with younger isogenic competitors upregulated both YAP1 and MYC targets relative to their non-competing controls (FIG. 21G) with a concomitant upregulation of ribosomal genes (FIG. 21I). This difference in their profiles may represent an intrinsic capacity to respond competitively when challenged, which mHTT-expressing HD hGPCs lack. Nonetheless, this upregulation was insufficient to match the greater fitness of their younger counterparts, which similarly—but to a relatively greater degree—manifested the selective upregulation of YAP1 and MYC targets, as well as ribosomal genes, relative to their non-competing controls (FIGS. 21G-H). Together, these data indicate that the determinants of relative cell fitness may be conserved across different scenarios of challenge, and that the outcomes of the resultant competition are heavily influenced by the relative ages of the competing populations.

### Example A9—Competitive Advantage is Linked to a Discrete Set of Transcription Factors

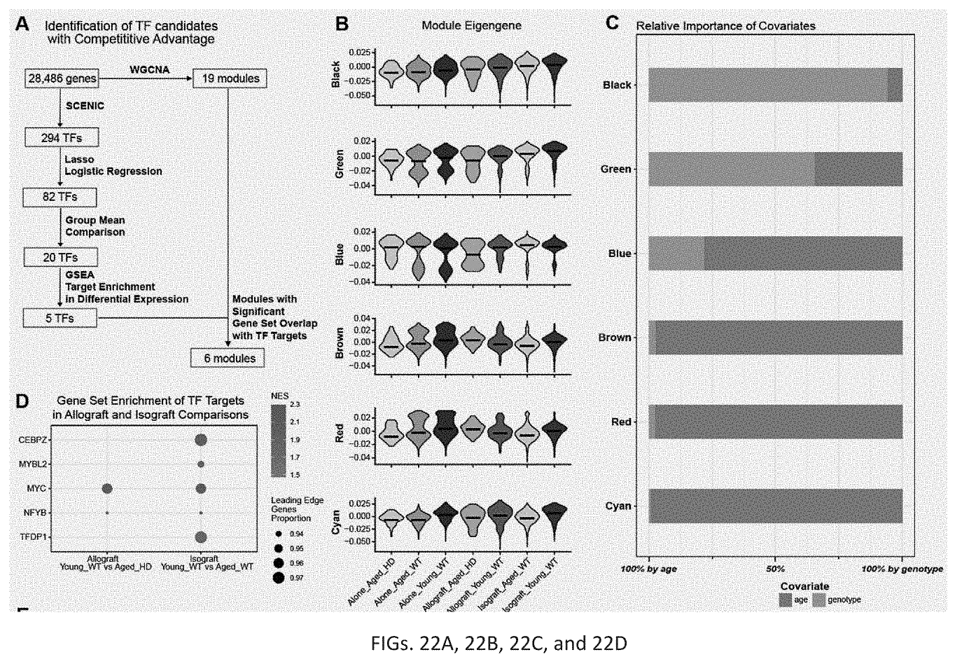

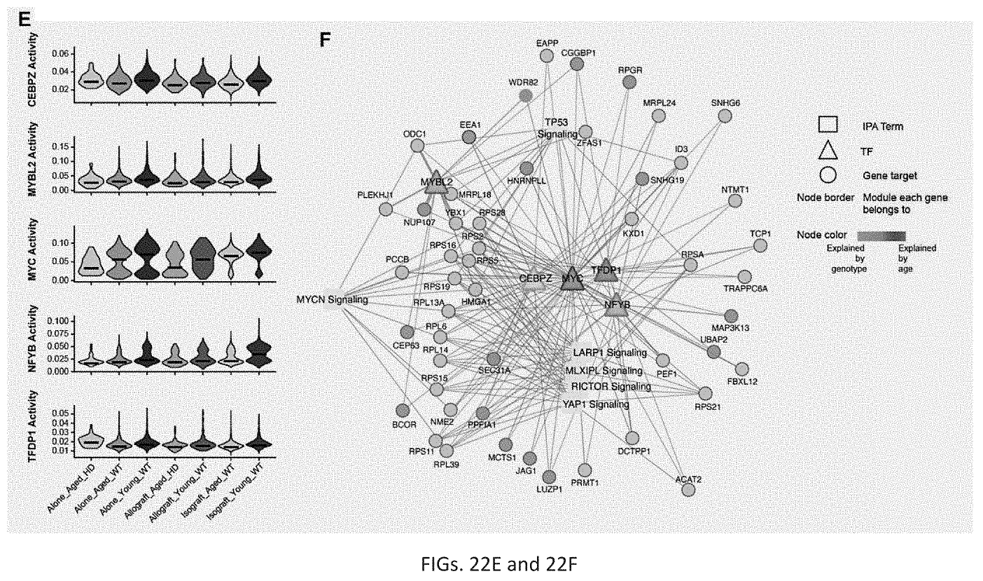

We next asked what gene signatures would define the competitive advantage of newly-transplanted human GPCs over resident cells. To that end, inventors applied a multi-stepped analysis using lasso-regulated logistic regression (FIG. 22A), that pinpointed 5 TFs (CEBPZ, MYBL2, MYC, NFYB, TFDP1) whose activities could significantly explain the dominance of young WT GPCs over both aged HD and aged WT GPCs. These 5 TFs and their putative targets established gene sets (regulons) which were upregulated (normalized enrichment score [NES]>0, adjusted p<10⁻²) in the young WT cells, in both the allograft and isograft models (FIG. 22D). It was also noticed that while their activities varied when not in a competitive environment (aged HD, aged WT, young WT alone), their mean activities were higher in the dominant young WT cells in both allograft (vs HD) and isograft (vs older isogenic self) paradigms, especially so for MYC (FIG. 22E).

Next, inventors set out to identify cohorts of genes with defined expression patterns, as well as significant overlaps to the five prioritized regulons above. Inventors first employed weighted gene co-expression network analysis (WGCNA) to detect a total of 19 modules in the GPC dataset (FIG. 22A). Six modules harbored genes with significant overlap to the targets of CEBPZ, MYBL2, MYC, NFYB, and TFDP1 (FIG. 22B). Inventors then asked if the expression pattern of prioritized modules could be explained by the age of cells (young vs. old), by their genotype (HD vs. WT), or both. WGCNA defines module eigengene as the first principal component of a gene cohort, representing thereby the general expression pattern of all genes within that module. As such, inventors built linear models where module eigengene was a response that was described by both age and genotype. It was observed that modules brown, red, and cyan were mostly influenced by age, while modules black, blue, and green were influenced by both age and genotype (FIG. 22C).

MYC, whose regulated pathway activation had already been inferred as conferring competitive advantage (FIGS. 20 and 21), was also one of the five prioritized TFs. Inventors thus further characterized the MYC regulon and its downstream targets, and noticed how these downstream targets were also regulated by other prioritized TFs (FIG. 22F). Interestingly, while MYC localized to module brown, a large proportion of its targets belonged to module blue. The blue module genes were similarly expressed in the non-competing control paradigms, but their expression levels were higher in the young WT compared to the aged HD in the WT vs HD allograft paradigm (FIG. 22B), a pattern suggesting that the blue signature was not activated unless cells were in a competing environment. Furthermore, inventors noted lower expression of these genes in the aged HD relative to the aged WT hGPCs (FIGS. 22E-F), which may highlight the intrinsically greater capacity of WT cells to compete, congruent with earlier observation that aged WT hGPCs respond differently than HD hGPCs when challenged with newly-engrafted WT GPCs. Importantly, the blue module eigengene could be described by both genotype and age, demonstrating that the competitive advantage associated with MYC signaling was driven by both of these variables. Accordingly, the targets in this network were enriched for pathways regulating cell proliferation (TP53, RICTOR, YAP), gene transcription (MYCN, MLXIPL), and protein synthesis (LARP1), each of which had been previously noted as differentially-expressed in each competitive scenario (FIGS. 20 and 21). As such, the output of this competition-triggered regulatory network appeared to confer competitive advantage upon young WT hGPCs when introduced into the adult brain, whether confronted by older HD-derived or isogenic hGPCs.

### Examples—Part B Rejuvenating Glial Progenitor Cell or a Progeny Thereof Experimental Models and Subject Details

Human Subjects

Details on fetal and adult brain samples are detailed in the methods section “Adult and Fetal Brain Processing for Cell Isolation.” The sex of fetal samples was not provided during tissue acquisition.

Cell Lines

The human iPSC line C27 was used to generate hGPCs in which predicted transcripts of interest were validated. The C27 line is male, and was obtained from Lorenz Studer. Cells were differentiated into GPCs as detailed in the methods section (see: Human iPSC-derived production of GPCs) (Chambers et al., 2009).

Methods Details

Adult and Fetal Brain Processing for Cell Isolation Human brain samples were obtained under approved Institutional Review Board protocols from consenting patients at Strong Memorial Hospital at the University of Rochester. Brain tissue was obtained from normal GW 18-24 cortical and/or VZ/SVZ dissections or adult white matter/cortex epileptic resections (18F,19M, and 27F years old for mRNA, 8M, 20F, 43M, and 54F years old for miRNA). Fetal GPC acquisition, dissociation and immunomagnetic sorting of A2B5⁺/PSA-NCAM⁻ cells were as described (Windrem et al., 2004). GPCs were isolated from dissociated tissue using a dual immunomagnetic sorting strategy: depleting mouse anti-PSA-NCAM⁺ (Millipore, DSHB) cells, using microbead tagged rat anti-mouse IgM (Miltenyi Biotech), then selecting A2B5⁺ (clone 105; ATCC, Manassas, Va.) cells from the PSA-NCAM⁻ pool, as described (Windrem et al., 2004; Windrem et al., 2008). After sorting, cells were maintained for 1-14 days in DMEM-F12/N1 with 10 ng/ml bFGF and 20 ng/ml PDGF-AA. Alternatively CD140a/PDGFαR-defined GPCs were isolated and sorted using MACS as described (Sim et al., 2011b), yielding an enriched population of CD140⁺ glial progenitor cells.

Bulk RNA-Sequencing

RNA was purified from isolates via Qiagen RNeasy kits and bulk RNA sequencing libraries were constructed. Samples were sequenced deeply on an Illumina HiSeq 2500 at the University of Rochester Genomics Research Center. Raw FASTQ files were trimmed and adapters removed using fastp (Chen et al., 2018) and aligned to GRCh38 using Ensembl 95 gene annotations via STAR in 2-pass mode across all samples (Dobin et al., 2013) and quantified with RSEM version (Li and Dewey, 2011). Subsequent analysis was carried out in R (R Core Team, 2017) where RSEM gene level results were imported via tximport (Soneson et al., 2015). DE analysis was carried out in DESeq2 (Love et al., 2014) where paired analyses (Fetal A2B5+ vs CD140a+, fetal CD140a+vs CD140a−) had paired information added to their models. For adult vs fetal DE analysis, age was concatenated with sort marker (CD140a− samples were not included) to define the group variable where sequencing batch was also added to the model to account for technical variability. Genes with an adjusted p-value<0.01 and an absolute log 2-fold change >1 were deemed significant. These data were then further filtered by meaningful abundance, defined as a median TPM (calculated via RSEM) of 1 in at least 1 group (20,663 genes met this criterion prior to DE).

scRNA-Seq Analysis

The fetal brain sample as processed as above for bulk rna-seq up until single cells were sorted via FACS for either CD140a⁺ or PSA-NCAM⁻/A2B5⁺ surface expression. Single cells were then captured on a 10× genomics chromium controller utilizing V2 chemistry and libraries generated according to manufacturer's instructions. Samples were sequenced on an Illumina HISEQ 2500 system. Demultiplexed samples were then aligned and quantified using Cell Ranger to an index generated from GRCh38 and Ensembl 95 gene annotations using only protein coding, lncRNA, or miRNA biotypes. Analysis of scRNA-Seq samples was carried out via Seurat (Butler et al., 2018) within R. Both samples were merged and low-quality cells filtered out as defined by having mitochondrial gene expression greater than 15% or having fewer than 500 unique genes. Samples were then normalized utilizing SCTRANSFORM taking care to regress out contributions due to total number of UMIs, percent mitochondrial gene content or the difference in S phase and G2M phase scores of each cell. PCA was then calculated, UMAP was run using the first 30 dimensions with n.neighbors=60 and repulsion.strength=0.8. FindNeighbors was then run followed by FindClusters with resolution set to 0.35. Based on expression profiles of each cluster, some similar clusters were merged into broader cell type clusters. Static differential expression of clusters was computed using the MAST test (Finak et al., 2015) where an adjusted p-value of <0.01 and an absolute log 2 fold change of >0.5 was deemed significant. Prediction of active transcription factor regulons was carried out with the SCENIC package in R (Aibar et al., 2017) using the hg38 databases located at resources.aertslab.org/cistarget/. Genes were included in co-expression analyses if they were expressed in at least 1% of cells.

Ingenuity Pathway Analysis and Network Construction

Differentially expressed genes were fed into Ingenuity Pathway Analysis (Qiagen) to determine significant canonical, functional, and upstream signaling terms. For construction of the IPA network, terms were filtered for adjusted p-values below 0.001. Non-relevant IPA terms were removed along with highly redundant functional terms assessed via jaccard similarity indices using the iGraph package (Csardi, 2006). Modularity was established within Gephi (Bastian et al., 2009) and the final network was visualized using Cytoscape (Shannon, 2003). Genes and terms of interest were retained for visualization purposes. Modules were broken out from one another and organized using the yFiles organic layout.

Inference of Transcription Factor Activity

Adult and fetal enriched gene lists were fed separately into RcisTarget (Aibar et al., 2017) to identify overrepresentation of motifs in windows around the genes' promoters (500 bp up/100 bp down and 10 kb up and 10 kb down). Transcription factors that were associated with significantly enriched motifs (NES>3) were then filtered by their significant differential expression in the input gene list. Within each window and gene list, only appropriate TF-gene interactions (Repressors downregulating genes and activators upregulating genes) were kept. Scanning windows were then merged to produce TF-gene edge lists of predicted fetal/adult repressors/activators. Inventors finally narrowed the TFs of interest to those primarily reported as solely activators or repressors in the literature.

miRNA Microarray Analysis

A2B5⁺ adult (n=3) and CD140a⁺ fetal (n=4) cell suspensions were isolated via MACS as noted above and their miRNA isolated using miRNeasy kits according to manufacturer instructions (QIAGEN). Purified miRNA was then prepared and profiled on Affymetrix GeneChip miRNA 3.0 Arrays as instructed by their standard protocol. Raw CEL files were then read into R via the oligo (Carvalho and Irizarry, 2010) package and samples were normalized via robust multi-array averaging (RMA). Probes were then filtered for only human miRNAs according to Affymetrix's annotation, and differential expression was carried out in limma (Ritchie et al., 2015) where significance was established at an adjusted p-value<0.01. Finally, differentially expressed miRNAs were surveyed across five independent miRNA prediction databases using MIRNATAP (Pajak M, 2020) with min_src set to 2 and method set to “geom”. Transcription factor regulation of miRNAs was carried out via querying the TrasmiR V2.0 database (Tong et al., 2019).

Exploratory Analysis and Visualization

PCA of bulk RNA-Seq or microarray samples was computed via prcomp with default settings on variance stabilized values of DESeq2 objects. PCAs were plotted via autoplot in the ggfortify package. Volcano plots were generated using EnhancedVolcano. Graphs were further edited or generated anew using ggplot2 and aligned using patchwork.

Human iPSC-Derived Production of GPCs

Human induced pluripotent stem cells (C27 (Chambers et al., 2009)) were differentiated into GPCs using a previously described protocol (Osipovitch et al., 2019; Wang et al., 2013; Windrem et al., 2017). Briefly, cells were first differentiated to neuroepithelial cells, then to pre-GPCs, and finally to GPCs. GPCs were maintained in glial media supplemented with T3, NT3, IGF1, and PDGF-AA.

Lentiviral Overexpression

For overexpression of E2F6, ZNF274, IKZF3, or MAX, inventors first identified the most abundant protein coding transcript of each of these genes from the adult hGPC dataset. cDNAs for each transcript were cloned downstream of the tetracycline response element promoter in the pTANK-TRE-EGFP-CAG-rtTA3G-WPRE vector. Viral particles pseudotyped with vesicular stomatitis virus G glycoprotein were produced by transient transfection of HEK293FT cells and concentrated by ultracentrifugation, and titrated by QPCR (qPCR Lentivirus Titer Kit, ABM-Applied Biological Materials Inc). iPSC (C27) derived GPC cultures (160-180 days in vitro) were infected at 1.0 MOI in glial media for 24 hours. Cells were washed with HBSS and maintained in glial media supplemented with 1 doxycycline (Millipore-Sigma St. Louis, Mo.) for the remainder of the experiment. Transduced hGPCs were isolated via FACS on DAPI⁻/EGFP⁺ expression 3, 7, and 10 days following the initial addition of doxycycline. Doxycycline control cells were sorted on DAPI⁻ alone.

Quantitative PCR

RNA from overexpression experiments was extracted using RNeasy micro kits (Qiagen, Germany). First-strand cDNA was synthesized using TaqMan Reverse Transcription Reagents (Applied Biosystems, USA). qPCR reactions were run in triplicate by loading 1 ng of RNA mixed with FASTSTART UNIVERSAL SYBRGREEN MASTERMIX (Roche Diagnostics, Germany) per reaction and analyzed on a real-time PCR instrument (CFX Connect Real-Time System thermocycler; Bio-Rad). Results were normalized to the expression of 18S from each sample.

Quantification and Statistical Analysis

For qPCR experiments, significant differences in delta CTs for each gene were analyzed in linear models constructed by the interaction of overexpression condition and timepoint with the addition of a cell batch covariate. Post hoc pairwise comparisons were tested via least-squares means tests against the Dox control within timepoints using the lsmeans package (Lenth, 2016). P-values were adjusted for multiple comparisons using the false discovery rate method whereby p-values<0.05 were deemed significant.

### Additional Resources

Bulk and scRNA-sequencing data from this paper and related previous publications can be explored in inventors' Shiny app at GlialExplorer.org or at ctngoldmanlab.genialis.corn.

### Example B1—CD140a Selection Enriches for Human Fetal Glial Progenitors More Efficiently than does A2B5

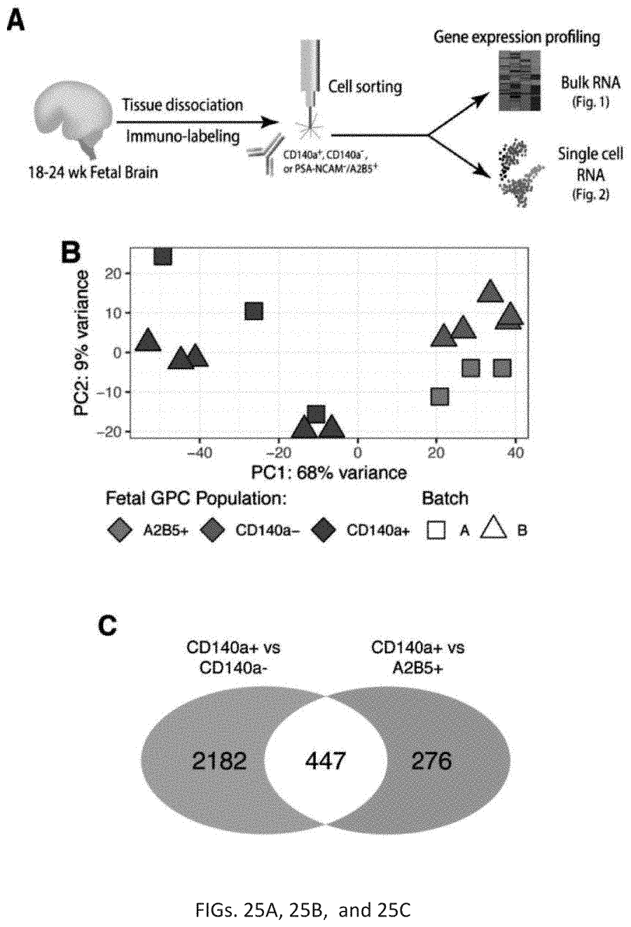

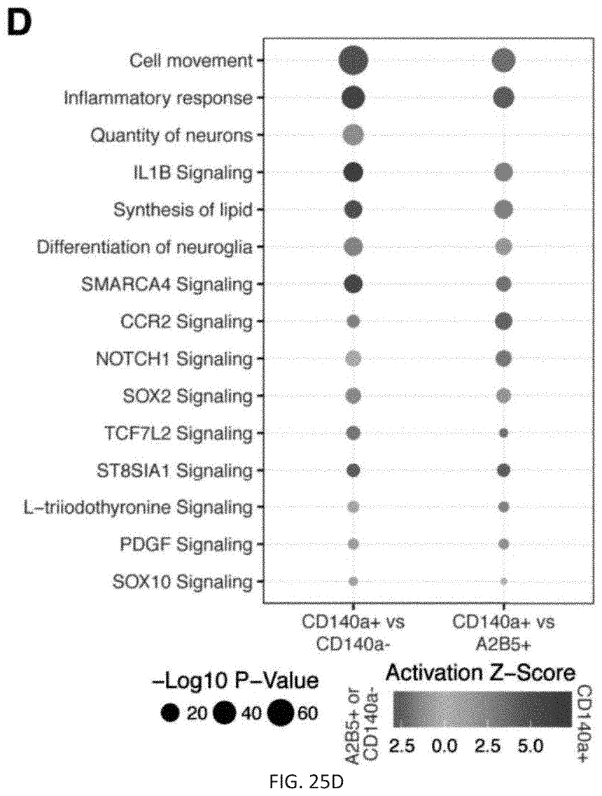

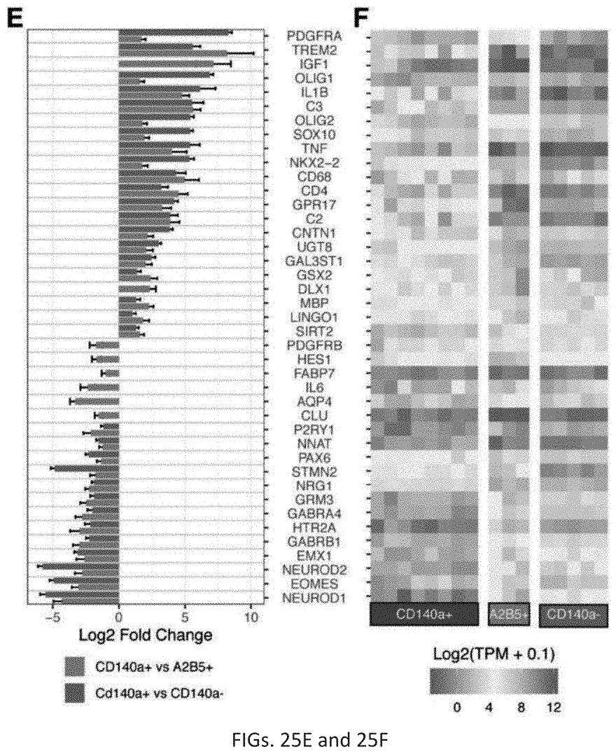

To identify the transcriptional concomitants to GPC aging, bulk and single cell RNA-Seq were first used to characterize hGPCs derived from second trimester fetal human tissue, whether isolated by targeting the CD140a epitope of PDGFRα, or the glial gangliosides recognized by monoclonal antibody A2B5. To that end, two sample-matched experiments were carried out whereby the ventricular/subventricular zones (VZ/SVZ) of 18-22 week gestational age (g.a.) fetal brains were dissociated and sorted via fluorescence activated cell sorting (FACS), for either CD140a+ and A2B5+/PSA-NCAM− (A2B5+) GPCs isolated from the same fetal brain (n=3), or for CD140a+ GPCs as well as the CD140a-depleted remainder (n=5; FIG. 25A).

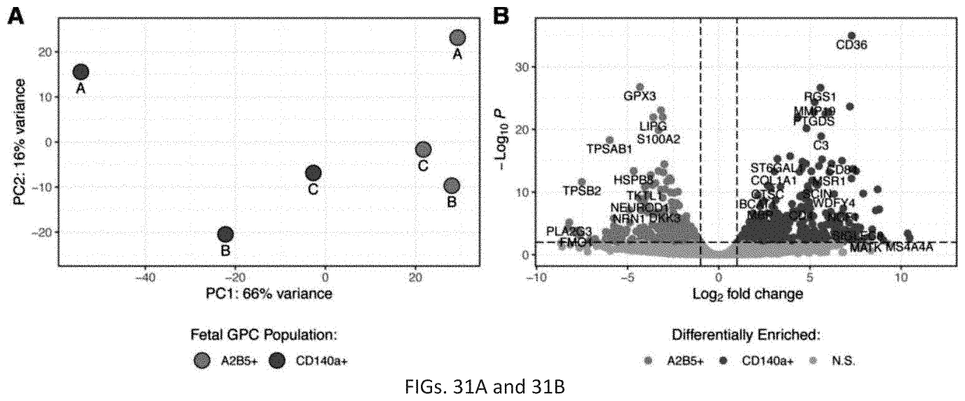

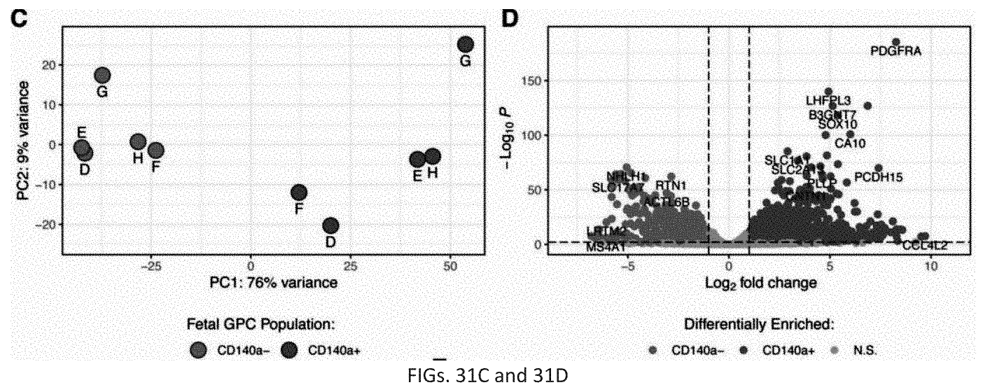

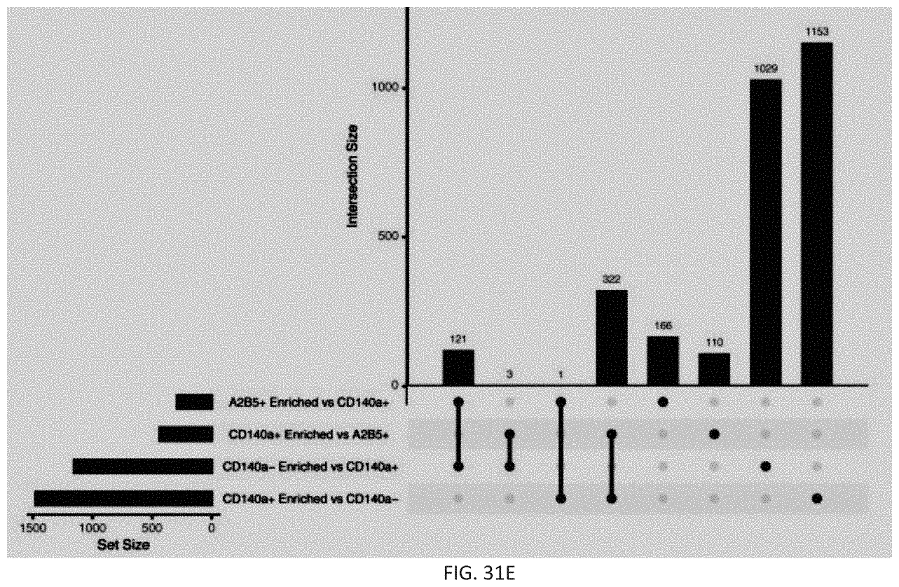

Bulk RNA-Seq libraries were then generated and deeply sequenced for both experiments. Principal component analysis (PCA) showed segregation of the CD140a+ and A2B5+ cells, and further segregation of both from the CD140a-depleted samples (FIG. 25B, FIGS. 31A-B). Differential expression in both paired cohorts (p<0.01, absolute log 2 fold change>1) identified 723 genes as differentially-expressed between CD140a+ and A2B5+ GPCs (435 in CD140a, 288 in A2B5). In contrast, 2,629 genes distinguished CD140a+ GPCs from CD140a− cells (FIG. 25C and FIGS. 31C-D). Differential gene expression directionality was highly consistent when comparing CD140+ to either A2B5+ or CD140− cells, with all but 4 genes being concordant (FIG. 31E).

Pathway enrichment analysis using Ingenuity Pathway Analysis (IPA) of both of these gene sets identified similar pathways as relatively active in CD140+ GPCs; these pathways included cell movement, oligodendroglial differentiation, lipid synthesis, and downstream PDGF, SOX10, and TCF7L2 signaling (FIG. 25). As expected, stronger activation Z-scores were typically observed when comparing CD140a+ GPCs to CD140a− cells rather than to A2B5+ GPCs. Interestingly, CD140a+ cells also differentially expressed a number of pathways related to the immune system, likely due to small amounts of microglial contamination as a result of re-expression of PDGFaR epitopes on the microglial surface. A2B5+ samples additionally displayed upregulated ST8SIA1, the enzyme responsible for A2B5 synthesis, as well as pro-neural pathways.

Among the genes differentially upregulated in CD140a+ isolates were PDGFRA itself, and a number of early oligodendroglial genes including OLIG1, OLIG2, NKX2-2, SOX10, and GPR17 (FIGS. 25E-F). Furthermore, the CD140a+ fraction also exhibited increased expression of later myelinogenesis-associated genes, including MBP, GAL3ST1, and UGT8. Beyond enrichment of the oligodendroglial lineage, many genes typically associated with microglia were also enriched in the CD140a isolates, including CD68, C2, C3, C4, and TREM2. In contrast, A2B5+ isolates exhibited enrichment of astrocytic (AQ4, CLU) and early neuronal (NEUROD1, NEUROD2, GABRG1, GABRA4, EOMES, HTR2A) genes, suggesting the expression of A2B5 by immature astrocytes and neurons as well as by GPCs and oligodendroglial lineage cells. Overall then, oligodendroglial enrichment was significantly greater in CD140a+ GPCs than A2B5-defined GPCs, when each was compared to depleted fractions, suggesting the CD140a isolates as being the more enriched in hGPCs, and thus CD140a as the more appropriate phenotype for head-to-head comparison with adult hGPCs.

### Example B2—Single Cell RNA-Sequencing Reveals Cellular Heterogeneity within Human Fetal GPC Isolates

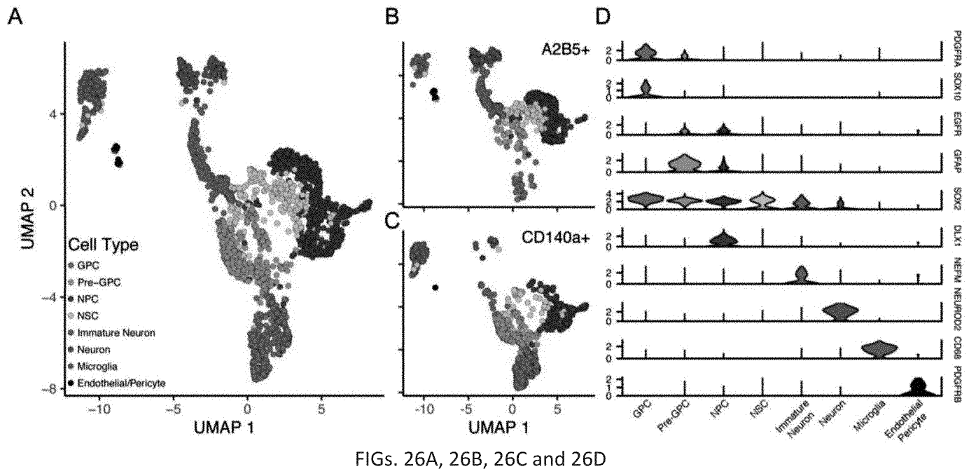

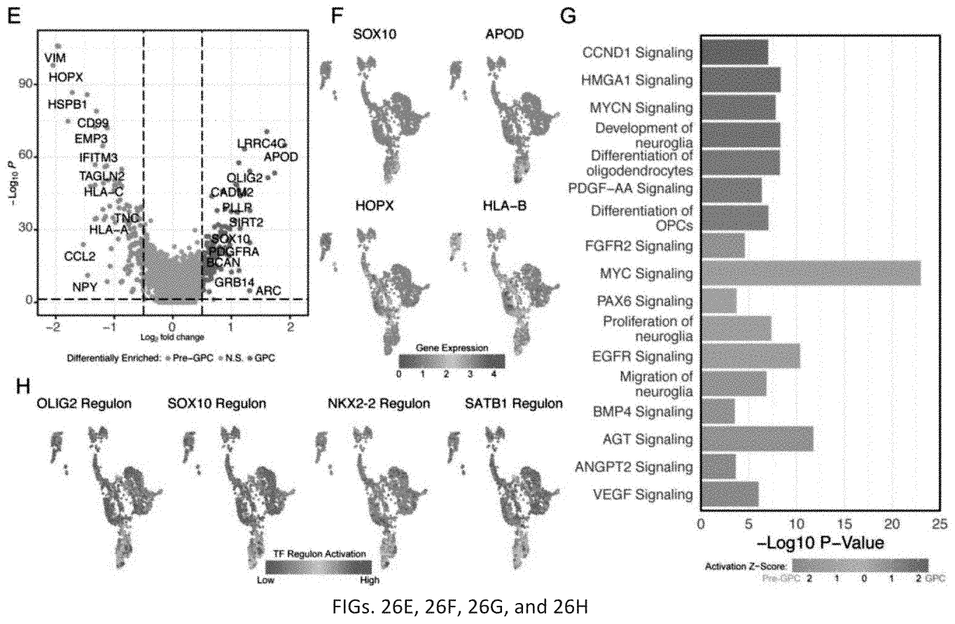

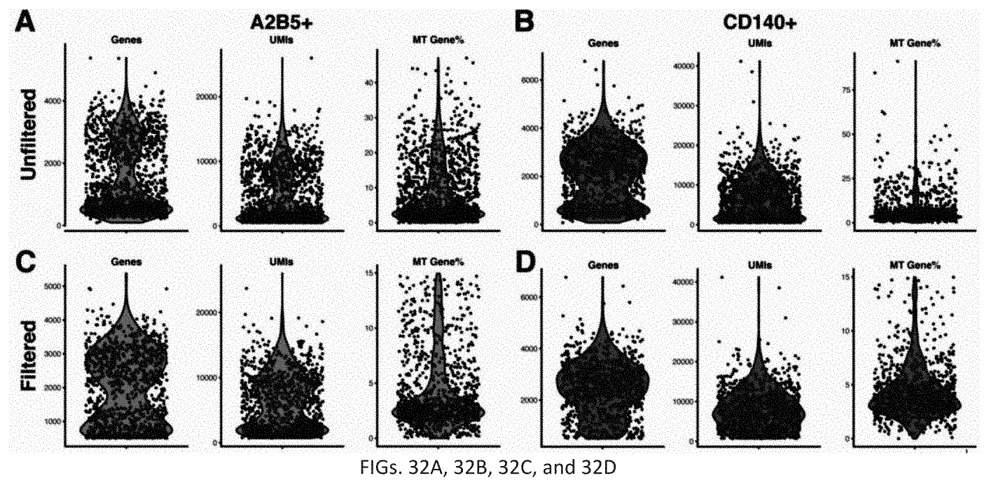

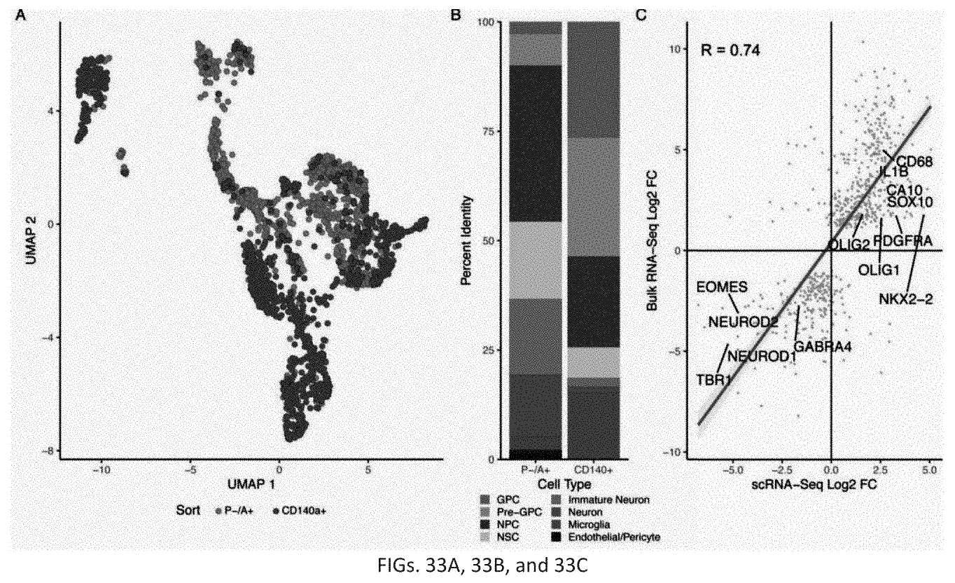

To further delineate the composition of fetal hGPC isolates at single cell resolution, inventors isolated both CD140a+ and A2B5+ hGPCs from 20-week g.a. fetal VZ/SVZ via FACS, and then assayed the transcriptomes of each by single cell RNA-Seq (FIG. 25A, 10× Genomics V2). Inventors sought to capture >1,000 cells of each; following filtration of low-quality cells (unique genes <500, mitochondrial gene percentage >15%), inventors were left with 1,053 PSA-NCAM−/A2B5+ and 957 CD140a+ high quality cells (median 6,845 unique molecular identifiers and 2,336 unique genes per cell; FIG. 32). Dimensional reduction via uniform manifold approximation and projection (UMAP), followed by shared nearest neighbor modularity-based clustering of all cells using Seurat (Butler et al., 2018), revealed 11 clusters with 8 primary cell types, as defined by their differential enrichment of marker genes. These primary cell types included: GPCs, pre-GPCs, neural progenitor cells (NPCs), immature neurons, neurons, microglia, and a cluster consisting of endothelial cells and pericytes. It was found that the CD140a+ FACS isolates were more enriched for GPC and pre-GPC populations than were the fetal A2B5+/PSA-NCAM− cells (FIGS. 26A-D, FIGS. 33A-C). Furthermore, whereas the CD140a-sorted cells were largely limited to GPCs and pre-GPCs, with only scattered microglial contamination, the A2B5+/PSA-NCAM− isolates also included astrocytes and neuronal lineage cells, the latter despite the upfront depletion of neuronal PSA-NCAM (FIG. 33A-C). These data supported the more selective and phenotypically-restricted nature of CD140a rather than A2B5-based GPC isolation.

On that basis, inventors next explored the gene expression profiles of the predominant cell populations in the CD140a₊ fetal isolates, GPCs and pre-GPCs (FIG. 33B). Differential expression between these two pools yielded 269 (143 upregulated, 126 down-regulated; p<0.01, log 2 fold change >0.5; FIG. 26E). During the pre-GPC to GPC transition, early oligodendroglial lineage genes were rapidly upregulated (OLIG2, SOX10, NKX2-2, PLLP, APOD), whereas those expressed in pre-GPCs effectively disappeared (VIM, HOPX, TAGLN2, TNC). Interestingly, genes involved in the human leukocyte antigen system, including HLA-A, HLA-B, HLA-C and B2M, were all downregulated as the cells transitioned to GPC stage (FIG. 26F). IPA analysis indicated that pre-GPCs were relatively enriched for terms related to migration, proliferation, and those presaging astrocytic identity (BMP4, AGT, and VEGF signaling), whereas GPCs displayed enrichment for terms associated with acquisition of an oligodendroglial identity (PDGF-AA, FGFR2, CCND1), in addition to activation of the MYC and MYCN pathways (FIG. 26G). Using single cell co-expression data together with promoter motif enrichment using the SCENIC package (Aibar et al., 2017), inventors then identified 262 transcription factors that were predicted to be relatively activated in GPCs vs pre-GPCs (Wilcoxon rank sum test, p<0.01). These included SATB1, as well as the early GPC specification factors OLIG2, SOX10, and NKX2-2 (FIG. 26H).

### Example B3—Human Adult and Fetal GPCs are Transcriptionally Distinct

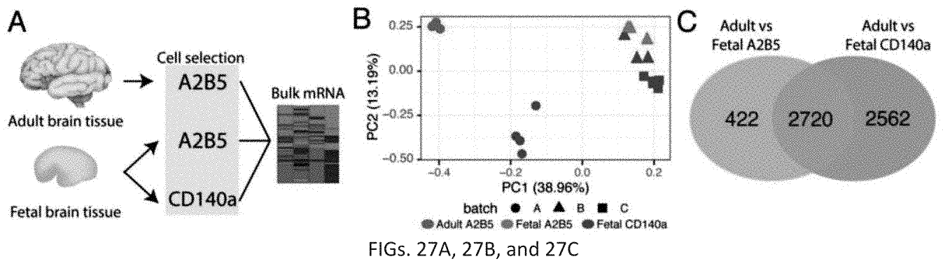

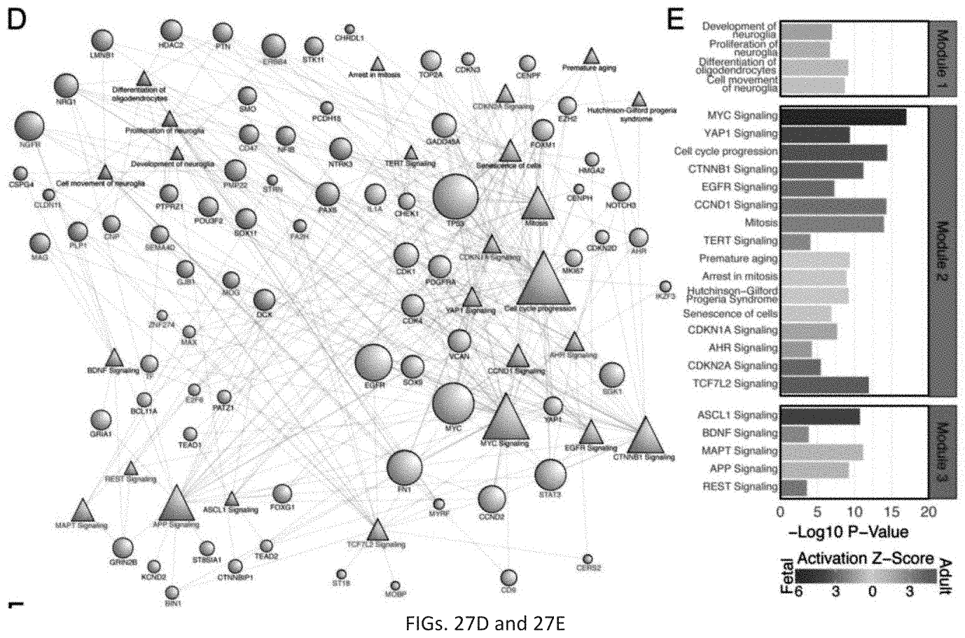

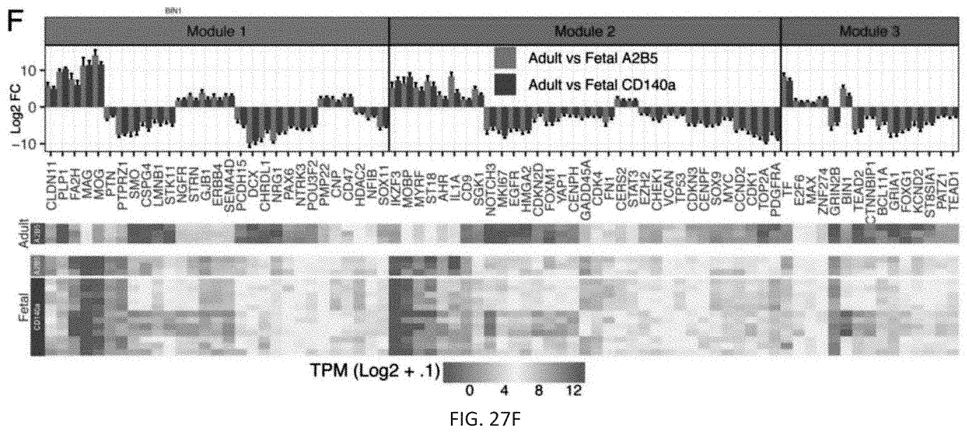

We next asked how adult hGPCs might differ in their transcription from fetal hGPCs. To this end, A2B5₊ hGPCs were isolated from surgically-resected adult human temporal neocortex (19-21 years old, n=3) and their bulk RNA expression assessed, as paired together with four additional fetal CD140a₊ samples. It was previously noted that A2B5 selection is sufficient to isolate GPCs from adult human brain, and is more sensitive than CD140a in that regard, given the maturation-associated down-regulation of PDGFRA expression in adult hGPCs (Sim et al., 2006; Windrem et al., 2004). Confirming that prior observation, it was found here that PDGFRA in A2B5₊ adult GPCs was expressed with a median TPM of 0.55, compared to a median TPM of 47.56 for fetal A2B5₊ cells. By pairing sequencing and analysis with fetal CD140a-selected cells, inventors enabled regression of sequencing batch effects while simultaneously increasing power (FIG. 27A). Depletion of PSA-NCAM₊ cells was not necessary for adult hGPC samples, as the expression of PSA-NCAM ceases in the adult cortex and white matter (Seki and Arai, 1993). As a result, PCA of human adult and fetal GPCs illustrated tight clustering of adult GPCs, sharply segregated from both sorted fetal hGPC pools (FIG. 27B). Differential expression of adult GPCs compared to either A2B5₊ or CD140a₊ fetal GPC populations yielded 3,142 and 5,282 significant genes, respectively (p<0.01; absolute log 2 fold-change >1) (FIG. 27C). To increase the accuracy of defining differential expression, downstream analyses were carried out on the intersecting 2,720 genes (FIG. 27D, 1,060 up-regulated and 1,660 down-regulated in adult GPCs compared to fetal hGPCs). Remarkably, within these two differentially-expressed gene sets, 100% of genes were directionally concordant.

To better understand the differences between adult and fetal GPCs, inventors next constructed a gene ontology network of non-redundant significant IPA terms and their contributing differentially-expressed genes (FIGS. 27D-E). Spin glass community detection of this network uncovered three modules (Modules M1-M3) of highly connected functional terms (FIG. 27E) and genes (FIG. 27F). M1 included terms and genes linked to glial development, proliferation, and movement. Notably, a number of genes associated with GPC ontogeny were downregulated in adult GPCs; these included CSPG4/NG2, PCDH15, CHRDL1, LMNB1, PTPRZ1, and ST8SIA1. In contrast, numerous genes whose appearance precedes and continues through oligodendrocyte differentiation and myelination were upregulated in adult GPCs, including MAG, MOG, MYRF, PLP1, CD9, CLDN11, CNP, ERBB4, GJB1, PMP22, and SEMA4D.

Module 2 harbored numerous terms associated with cellular aging and the modulation of proliferation and senescence. Cell cycle progression and mitosis were predicted to be activated in fetal GPCs due to strong enrichment of proliferative factors including MKI67, TOP2A, CENPF, CENPH, CHEK1, EZH2 and numerous cyclins, including CDK1 and CDK4. Furthermore, proliferation-inducing pathways were also inferred to be activated; these included MYC, CCND1, and YAP1 signaling, of which both YAP1 and MYC transcripts were similarly upregulated. In that regard, transient overexpression of MYC in aged rodent GPCs has recently been shown to restore their capacity to both proliferate and differentiate. Conversely, adult GPCs exhibited an upregulation of senescence-associated transcripts, including E2F6, MAP3K7, DMTF1/DMP1, OGT, AHR, RUNX1, and RUNX2. At the same time, adult hGPCs exhibited a down-regulation of fetal transcripts that included LMNB1, PATZ1, BCL11A, HDAC2, FN1, EZH2, and YAP1 and its cofactor TEAD1. As a result, functional terms predicted to be active in adult hGPCs included senescence, the rapid onset of aging observed in Hutchinson-Gilford progeria, and cyclin-dependent kinase inhibitory pathways downstream of CDKN1A/p21 and CDKN2A/p16. Furthermore, AHR and its signaling pathway, which has been implicated in driving senescence via the inhibition of MYC, was similarly upregulated in adult GPCs.

Module 3 consisted primarily of developmental and disease linked signaling pathways that have also been associated with aging. This included the predicted activation of ASCL1 and BDNF signaling in fetal hGPCs and MAPT/Tau, APP, and REST signaling in adult GPCs. Overall, the transcriptional and functional profiling of adult GPCs revealed a reduction in transcripts associated with proliferative capacity, and a shift toward senescence and more mature phenotype.

### Example B4—Inference of Transcription Factor Activity Implicates Adult GPC Transcriptional Repressors

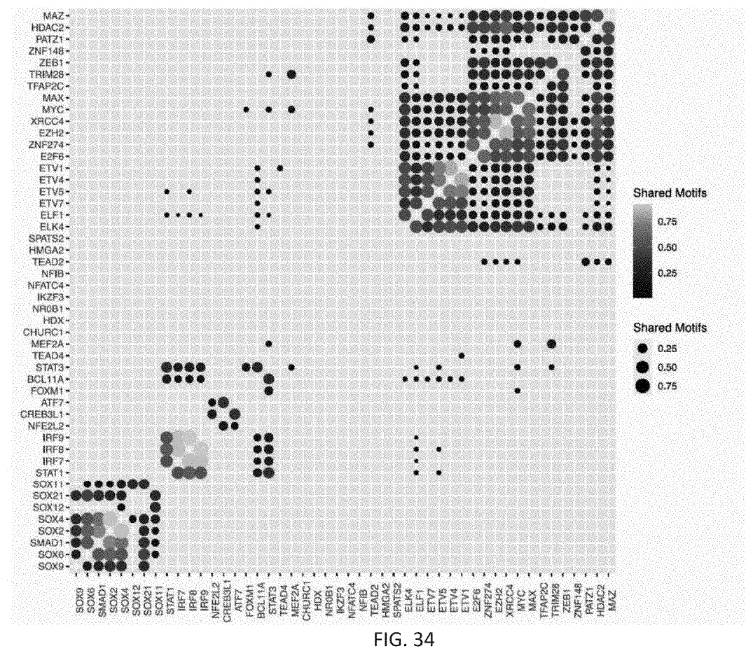

Given the significant transcriptional disparity between adult and fetal GPCs, inventors next asked whether inventors could infer which transcription factors direct their identities. To accomplish this, inventors first scanned two promoter windows (500 bp up/100 bp down, 10 kb up/10 kb down) of adult or fetal enriched GPC gene sets to infer significantly enriched TF motifs. This identified 48 TFs that were also differentially-expressed in the scanned intersecting dataset (FIG. 34). Among these, inventors focused on TFs whose primary means of DNA interaction were exclusively either repressive or stimulatory, while also considering the enrichment of their known cofactors. This analysis yielded 12 potential upstream regulators to explore (FIGS. 28A-C): 4 adult repressors, E2F6, ZNF274, MAX, and IKZF3; 1 adult activator, STAT3; 3 fetal repressors, BCL11A HDAC2, and EZH2; and 4 fetal activators, MYC, HMGA2, NFIB, and TEAD2. Interestingly, of these predicted TFs, 3 groups shared a high concordance of motif similarity within their targeted promoters: 1) E2F6, ZNF274, MAX, and MYC; 2) STAT3 and BCL11A; and 3) EZH2 and HDAC2, suggesting that they may cooperate or compete for DNA binding at shared loci (FIG. 28A and FIG. 34).

We next constructed four potential signaling pathways based on curated transcriptional interactions, to predict those genes targeted by the set of TFs (FIGS. 28D-G). Among activators enriched in fetal GPCs (FIG. 28D), MYC, a proliferative factor, NFIB, a key determinant of gliogenesis, TEAD2, a YAP/TAZ effector, and HMGA2, another proliferative factor, were each predicted to activate cohorts of progenitor stage genes, including both mitogenesis-associated transcripts and those demonstrated to inhibit the onset of senescence. Direct positive regulation was also predicted between these four fetal activators, with NFIB being driven by HMGA2 and TEAD2, MYC being driven by TEAD2 and NFIB, HMGA2 being driven by MYC and TEAD2, and TEAD2 being reciprocally driven by MYC (FIG. 28D). In contrast to these fetal activators, fetal stage repressors, including the C2H2 type zinc finger BCL11A, the polycomb repressive complex subunit EZH2, and histone deacetylase HDAC2, were each predicted to repress more mature oligodendrocytic gene expression at this stage (FIG. 28E). Furthermore, all three of these TFs were predicted to inhibit targets implicated in senescence. As such, these factors appear to directly orchestrate downstream transcriptional events leading to maintenance of the cycling progenitor state.

We next assessed these predicted adult GPC signaling networks for a potential mechanism responsible for their age-related gene expression changes. STAT3 was predicted to shift GPC identity towards glial maturation via the upregulation of a large cohort of early differentiation- and myelination-associated oligodendrocytic genes (FIG. 28F). In addition, STAT3 was also inferred to activate a set of senescence-associated genes including BIN1, RUNX1, RUNX2, DMTF1, CD47, MAP3K7, CTNNA1, and OGT. At the same time, repression in adult GPCs was predicted to be effected through the Ikaros family zinc finger IKZF3/Aiolos, the KRAB (kruppel associated box) zinc finger ZNF274, the MYC-associated factor MAX, and cell cycle regulator E2F6 (FIG. 28G) Targeting by this set of transcription factors predicted repression of those gene sets contributing to the fetal GPC signature, and this was indeed observed in the down-regulation of the early progenitor genes PDGFRA and CSPG4, as well as of the cell cyclicity genes CDK1, CDK4, and MKI67. Repression of YAP1, LMNB1, and TEAD1, whose expression slows or prevents the onset of senescence, was also predicted. Interestingly, this set of four adult repressors predicted the down-regulated expression of each of the fetal enriched activators NFIB, MYC, TEAD2, and HMGA2, in addition to the fetal enriched repressors BCL11A, EZH2, and HDAC2.

### Example B5—Expression of Adult-Enriched Repressors Induces Age-Associated Transcriptional Changes in GPCs

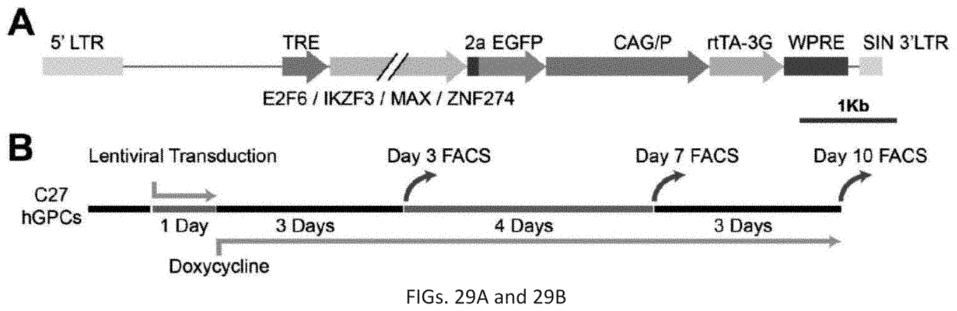

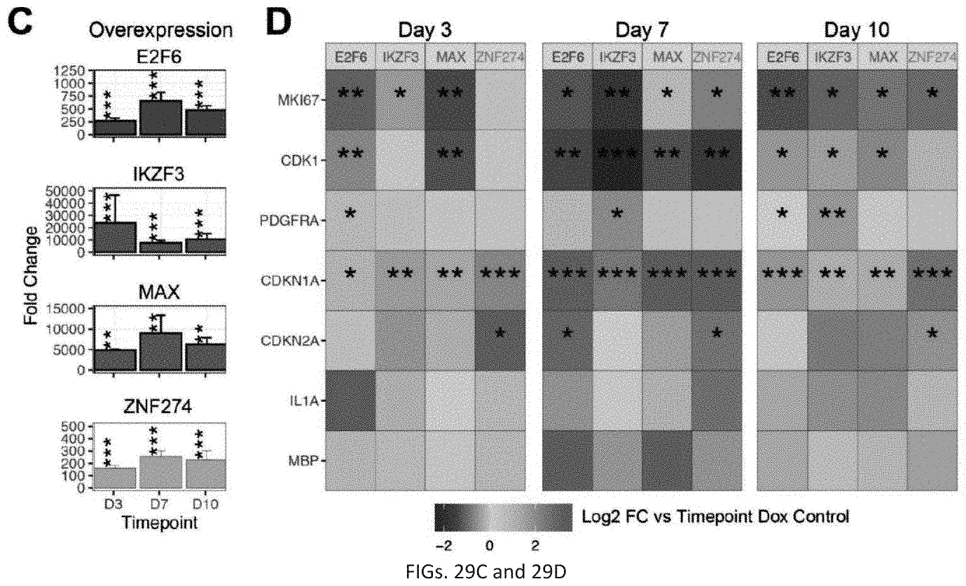

We next asked whether the four adult-enriched transcriptional repressors inventors identified in FIG. 28G, E2F6, IKZF3, MAX, and ZNF274, were individually sufficient to induce aspects of the age-associated changes in gene expression by otherwise young GPCs. To accomplish this, inventors designed doxycycline (Dox) inducible overexpression lentiviruses for each transcription factor (FIG. 29A).

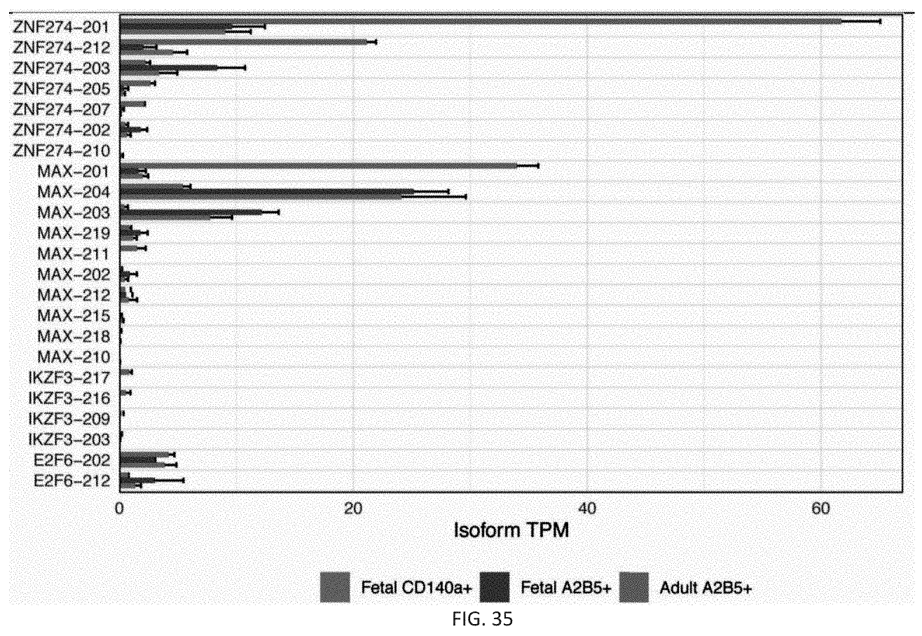

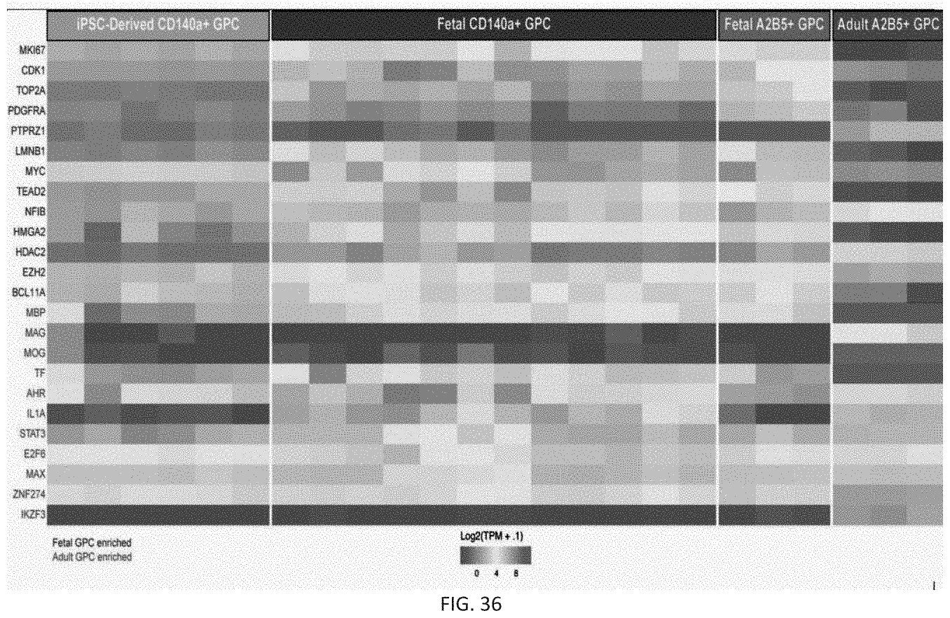

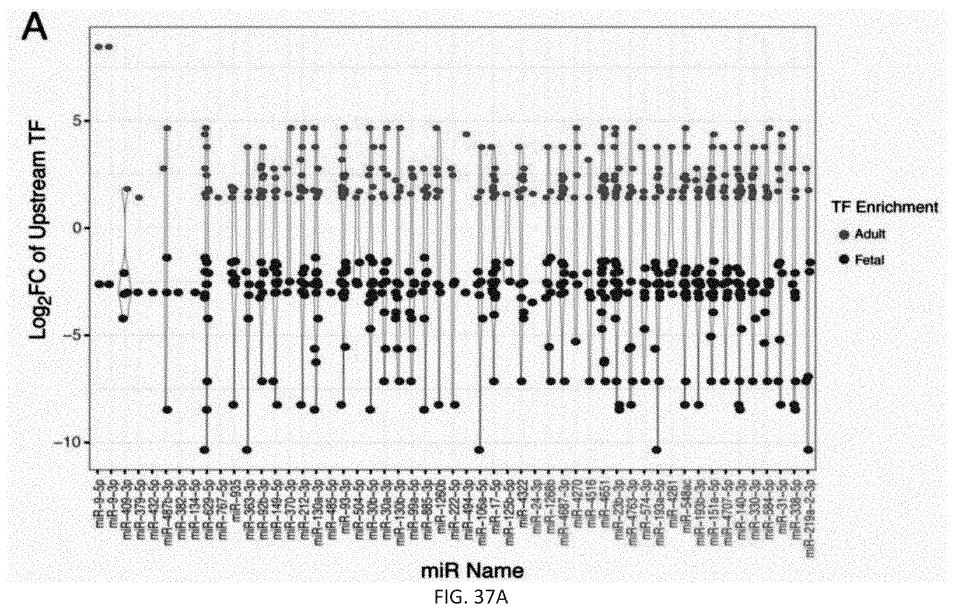

Briefly, inventors first identified which protein-coding isoform was most abundant in adult GPCs for each repressor, so as to best mimic endogenous age-associated upregulation; these candidates were E2F6-202, IKZF3-217, MAX-201, and ZNF274-201 (FIG. 35). These cDNAs were cloned downstream of a tetracycline response element promoter, and upstream of a T2A self-cleaving EGFP reporter (FIG. 29A). Human induced pluripotent stem cell (iPSC)-derived hGPC cultures, prepared from the C27 line as previously described in Wang et al., 2013, Cell Stem Cell 12, 252-264 were then infected for 24 hrs, and then treated with Dox to induce transgene overexpression. C27 iPSC-derived GPCs were chosen as their transcriptome resembles that of fetal GPCs (FIG. 36), and they are similarly capable of engrafting and myelinating dysmyelinated mice upon transplantation. Over-expressing cells were selected via FACS for EGFP expression, at 3, 7, and 10 days following Dox addition (FIG. 29B, n=3-5). Uninfected cultures given Dox were used as controls.

RNA was extracted and aging-associated genes of interest were analyzed by qPCR. Significant induction of each adult-enriched repressor was observed at each timepoint following Dox supplementation (FIG. 29C). MKI67 and CDK1, genes whose upregulation are associated with active cell division, were significantly repressed at two or more timepoints in each over-expression paradigm (FIG. 29D). This was consistent with their diminished expression in adult GPCs (FIG. 27F), and suggested their direct repression by E2F6, MAX, and ZNF274 (MKI67), or by all four (CDK1). The GPC stage marker PDGFRA, the cognate receptor for PDGF-AA, was also significantly repressed at two timepoints in the IKZF3-transduced GPCs, as well as in the E2F6-transduced GPCs at day 3, consistent with its repression in normal adult GPCs. Interestingly, the senescence-associated cyclin-dependent kinase inhibitor CDKN1A/p21 was upregulated in response to each of the tested repressors at all timepoints, while CDKN2A/p16 was similarly upregulated in at all timepoints in ZNF274-transduced hGPCs, as well as in the E2F6-over-expressing GPCs at day 7 (FIG. 29D). In addition, MBP and ILIA, both of which are strongly upregulated in adult hGPCs relative to fetal, both exhibited sharp trends towards upregulated expression in response to repressor transduction, although timepoint-associated variability prevented their increments from achieving statistical significance. Together, these data supported the prediction that forced, premature expression of the adult-enriched GPC repressors, E2F6, IKZF3, MAX, and ZNF274, are individually sufficient to induce multiple features of the aged GPC transcriptome in young, iPSC-derived GPCs.

### Example B6—the miRNA Expression Pattern of Fetal hGPCs Predicts their Suppression of Senescence

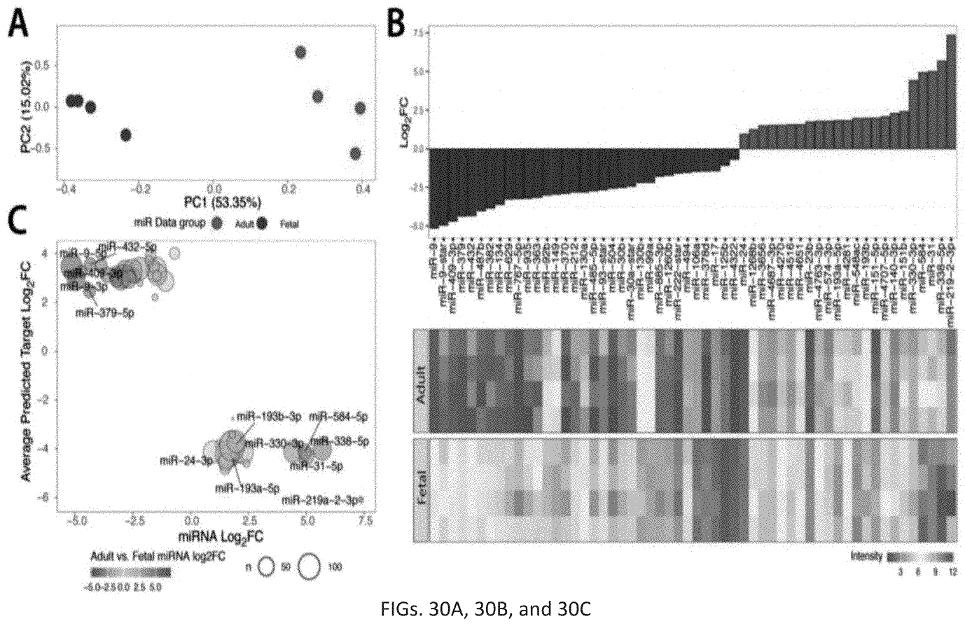

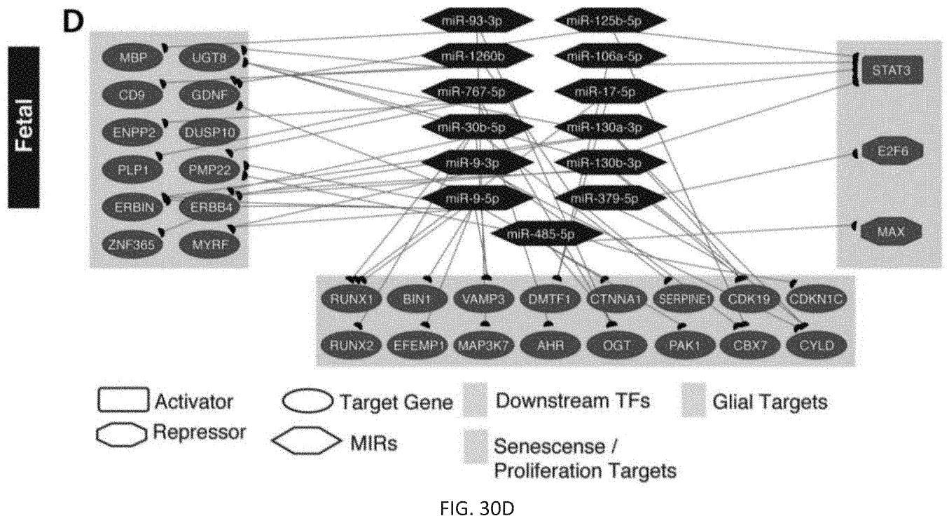

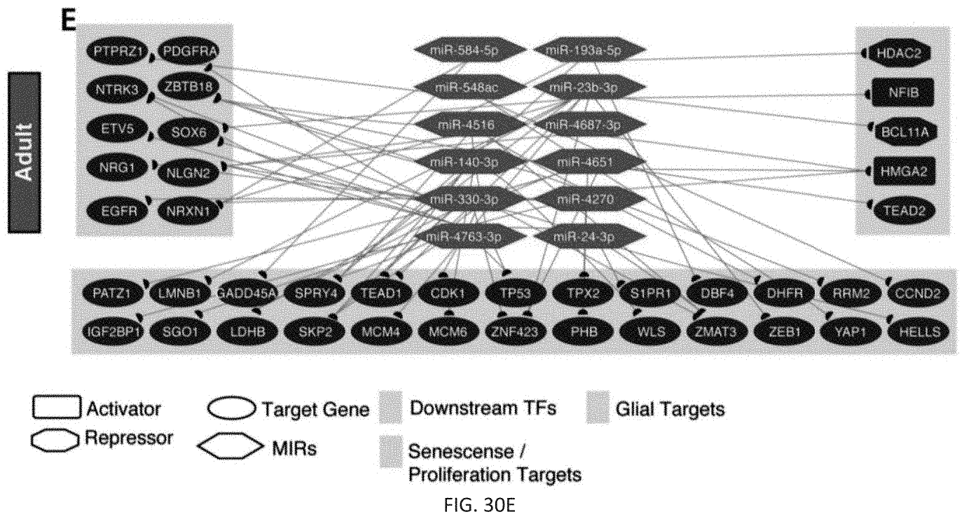

To identify potential post-transcriptional regulators of gene expression, inventors assessed differences in miRNA expression between adult and fetal GPCs (n=4) utilizing Affymetrix GeneChip miRNA 3.0 arrays. PCA displayed segregation of both GPC populations as defined by their miRNA expression profiles (FIG. 30A). Differential expression between both ages (adjusted p-value <0.01) yielded 56 genes (23 enriched in adult GPCs, 33 enriched in fetal GPCs, FIG. 30B-C). Notably among these differentially expressed miRNAs were the adult oligodendrocyte regulators miR-219a-3p and miR-338-5p (Dugas et al., 2010; Wang et al., 2017) in addition to fetal progenitor stage miRNAs miR-9-3p, miR-9-5p (Lau et al., 2008), and miR-17-5p (Budde et al., 2010).

We next utilized this cohort of miRNAs to predict genes whose expression might be expected to be repressed via miRNA upregulation, separately analyzing both the adult and fetal GPC pools. To accomplish this, miRNAtap was used to query five miRNA gene target databases: DIANA (Maragkakis et al., 2011), Miranda (Enright et al., 2003), PicTar (Lall et al., 2006), TargetScan (Friedman et al., 2009), and miRDB (Wong and Wang, 2015). To maximize precision, genes were only considered a target if they appeared in at least two databases. Among fetal-enriched miRs, this approach predicted an average of 36.3 (SD=24.5) repressed genes per miRNA. In contrast, among adult hGPC-enriched miRNAs, an average of 46.4 (SD=37.8) genes were predicted as targets per miRNA (FIG. 30C). Altogether, this identified the potential repression of 48.8% of adult GPC-enriched genes via fetal miRNAs, and repression of 39.9% of fetal GPC-enriched genes by adult miRNAs.

To assess the functional importance of these miRNA-dependent post-transcriptional regulatory mechanisms, inventors curated fetal and adult networks according to miRNA targeting of functionally-relevant, differentially expressed genes (FIGS. 30D-E). Proposed upstream adult transcriptional regulators STAT3, E2F6, and MAX were predicted to be inhibited via 7 miRNAs in fetal GPCs (FIG. 30D); these included the already-validated repression of STAT3 in other cell types by miR-126b-5p, miR-106a-5p, miR-17-5p, miR-130a-3p, and miR-130b-3p (Du et al., 2014a; Jiang et al., 2020; Zhang et al., 2020; Zhang et al., 2013; Zhao et al., 2013). In parallel, a number of early and mature oligodendrocytic genes were concurrently targeted for inhibition, all consistent with maintenance of the progenitor state; these included MBP, UGT8, CD9, PLP1, MYRF, and PMP22 (Goldman and Kuypers, 2015). Importantly, a cohort of genes linked to either the induction of senescence or inhibition of proliferation, or both, were also predicted to be actively repressed in fetal GPCs. These included RUNX1, RUNX2, BIN1, DMTF1/DMP1, CTNNA1, SERPINE1, CDKN1C, PAK1, IFI16, EFEMP1, MAP3K7, AHR, OGT, CBX7, and CYLD (Eckers et al., 2016; Elliott et al., 1999; Ferrand et al., 2015; Hu et al., 2019; Inoue and Sherr, 1998; Jiang et al., 2017; Kilbey et al., 2007; Lee and Zhang, 2016; Li et al., 2015; Mademtzoglou et al., 2018; Mikawa et al., 2014; Ni et al., 2017; Wotton et al., 2004; Xin et al., 2004; Zhang and Guo, 2018). Inhibition of senescence or activation of proliferation have also been noted by several of the miRNAs identified here, including miR-17-5p, miR-93-3p, miR-1260b, miR-106a-5p, miR-767-5p, miR-130a-3p, miR-9-3p, miR-9-5p, and miR-130b-3p (Borgdorff et al., 2010; Gao et al., 2019; Meng et al., 2017; O'Loghlen et al., 2015; Shen et al., 2015; Su et al., 2018; Tai et al., 2020; Wang et 2020a; Xia et al., 2019; Zhang and Guo, 2018). Together, these data provide a complementary mechanism by which fetal hGPCs may maintain their characteristic progenitor transcriptional state and signature.

### Example B7—Adult miRNA Signaling May Repress the Proliferative Progenitor State and Augur Senescence

We next inspected the potential miRNA regulatory network within adult hGPCs (FIG. 30E). This implicated five miRNAs controlling five identified active fetal transcriptional regulators including HDAC2, NFIB, BCLL1A, TEAD2, and HMGA2, whose silencing via miR-4651 has previously been shown to inhibit proliferation (Han et al., 2020). This cohort of miRNAs were predicted to operate in parallel to adult transcriptional repressors in inhibiting expression of genes involved in maintaining the GPC progenitor state including PDGFRA, PTPRZ1, ZBTB18, SOX6, EGFR, and NRXN1. Furthermore, the adult miRNA environment was predicted to repress numerous genes known to induce a proliferative state or to delay senescence, including LMNB1 (Freund et al., 2012), PATZ1 (Cho et al., 2012), GADD45A (Hollander et al., 1999), YAP1 and TEAD1 (Xie et al., 2013), CDK1 (Diril et al., 2012), TPX2 (Rohrberg et al., 2020), S1PR1 (Liu et al., 2019), RRM2 (Aird et al., 2013), CCND2 (Bunt et al., 2010), SGO1 (Murakami-Tonami et al., 2016), MCM4 and MCM6 (Mason et al., 2004), ZNF423 (Hernandez-Segura et al., 2017), PHB (Piper et al., 2002), WLS (Poudel et al., 2020), and ZMAT3 (Kim et al., 2012). More directly, induction of senescence or inhibition of proliferation has been linked to the upregulation of miR-584-5p (Li et al., 2017), miR-193a-5p (Chen et al., 2016), miR-548ac (Song et al., 2020), miR-23b-3p (Campos-Viguri et al., 2020), miR-140-3p (Wang et al., 2020b), and miR-330-3p (Wang et al., 2020b). Taken together, these data implicate these miRs as active participants in maintenance of the progenitor state in fetal hGPCs, and their modulation as a likely mechanism by which adult hGPCs assume their signatory gene expression profile.

### Example B8—Transcription Factor Regulation of miRNAs Establishes and Consolidates GPC Identity

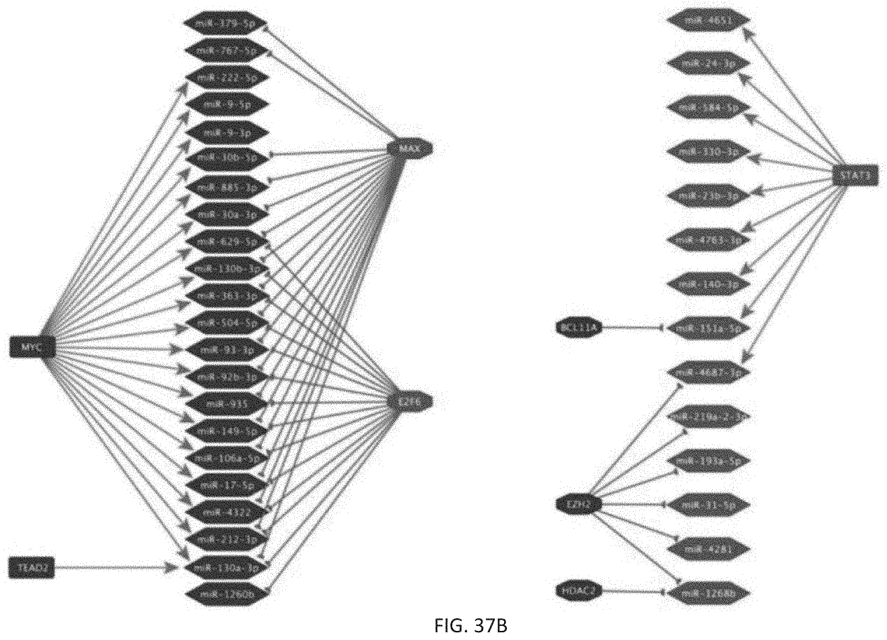

We next sought to predict the upstream regulation of differentially expressed miRNAs in fetal and adult GPCs by querying the TransmiR transcription factor miRNA regulation database (Tong et al., 2019). This approach predicted regulation of 54 of 56 of age-specific GPC miRNAs via 66 transcription factors that were similarly determined to be significantly differentially expressed between fetal and adult GPCs (FIG. 37A). Interestingly, the top four predicted miRNA-regulating TFs were all MYC-associated factors including MAX, MYC itself, E2F6, and the fetal enriched MYC associated zinc finger protein, MAZ, targeting 36, 33, 30, and 28 unique differentially expressed miRNAs respectively.

Inspection of proposed relationships in the context of 12 TF candidates (FIG. 28) indicated a large number of fetal hGPC-enriched miRNAs that were predicted to be targeted by both fetal activators and adult repressors, whereas those miRNAs enriched in adult GPCs were more uniquely targeted (FIG. 37B). MYC was predicted to drive the expression of numerous miRNAs in fetal GPCs, many of which were predicted to be repressed in adulthood via E2F6, MAX or both. miR-130a-3p in particular was predicted to be targeted by MYC, MAX, and E2F6, in addition to activation via TEAD2. Notably among validated TF-miRNA interactions in other cell types, the upregulation of the rejuvenating miR-17-5p by MYC, and its repression by MAX (Du et al., 2014b; Hackl et al., 2010; Ji et al., 2011; O'Donnell et al., 2005), has been reported. Similarly, the parallel activation of the proliferative miR-130-3p by MYC or TEAD2 and YAP1 (Shen et al., 2015; Wang et al., 2020a; Yang et al., 2013), has been reported, as has the activation of both arms of miR-9 by MYC (Ma et al., 2010a), which decreases with oligodendrocytic maturity (Lau et al., 2008).

In adult GPCs, enriched miRNAs predicted to be regulated by the significantly enriched TF cohort were more likely to be only targeted by an adult activator of fetal repressor with only miR-151a-5p and miR-468′7-3p, a predicted inhibitor of HMGA2, being targeted in opposition by STAT3 versus BCL11A and EZH2 respectively. Beyond this, miR-1268b was predicted to be inhibited by both EZH2 and HDAC2 in parallel. Notably, key oligodendrocytic microRNA, miR-219a-2-3p was predicted to remain inhibited in fetal GPCs via EZH2, whereas STAT3 likely drives the expression of 7 other miRs independently. Interestingly, STAT3, whose increased activity has been linked to senescence (Kojima et al., 2013), was also predicted to drive the expression of a cohort of miRNAs independently associated with the induction of senescence, including miR-584-5p, miR-330-3p, miR-23b-3p, and miR-140-3p.

Through integration of transcriptional and miRNA profiling, pathway enrichment analyses, and target predictions, inventors propose a model of human GPC aging whereby fetal hGPCs maintain progenitor gene expression, activate proliferative programs, and prevent senescence, while repressing oligodendrocytic and senescent gene programs both transcriptionally, and post-transcriptionally via microRNA. With adult maturation and the passage of time as well as of population doublings, hGPCs begin to upregulate repressors of these fetal progenitor-linked networks, while also activating programs to further a progressively more differentiated and ultimately senescent phenotype.

### Example B9—Glial Explorer: An Interactive Database to Query Human Glial Transcriptional Expression

As a resource to other researchers, inventors have developed a Shiny app (Chang et al.) (Accessible at GlialExplorer.org), that comprises a database describing human glial gene expression, including both bulk and scRNA-Sequencing datasets, as covered both here and in inventors' previous studies. This includes profiles of healthy human embryonic stem cell (hESC)-derived GPCs and astrocytes as well as those from Huntington's Disease cells (Osipovitch et al., 2019), healthy induced pluripotent stem cell (iPSC) derived GPCs and astrocytes along with those from schizophrenic patients (Windrem et al., 2017), and remyelinating or resting fetal-derived GPCs sorted out of immunodeficient chimeric mice (Windrem et al., 2020). Briefly, Glial Explorer allows simple querying of gene abundances (across all of the aforementioned datasets. Furthermore, abundance of splice variants can also be explored. Lastly, scRNA-Seq data can similarly be detailed through the generation of feature and violin plots. The intention is that this app and its included database should enable interested researchers to quickly survey their genes of interest, and to interactively assess their regulation and roles in human glial ontogeny and aging. More detailed expression profiling information is hosted at the genomics database available at ctngoldmanlab.genialis.com.

### Discussion

Human glial progenitors first appear in the 2^{nd} trimester of human development, after which a pool remains throughout the entirety of life. In early development and youth, these progenitors are highly proliferative and self-renewing. Yet their ability to divide and replenish lost myelin decreases substantially with age, as well as in the setting of antecedent demyelination and white matter disease. Given the evolutionary divergence between murine and human glia, it is important to interrogate human glia when assessing the basis for this loss of expansion potential, so as to identify the most therapeutically relevant targets. As such, inventors adopted a bulk RNA-sequencing strategy of FACS and MACS isolated fetal and adult human GPCs, together with scRNA-sequencing of fetal GPCs directly isolated from human brain, to more accurately track divergent transcriptional changes in the population of interest, while combatting potential off-target cell-type contaminants. This provided a set of genes whose expression distinguished human fetal GPCs from their aged successors, and which suggested a progressive bias towards early and terminal oligodendrocytic differentiation. This observation is in accordance with previous rodent GPC gene expression and proteome data noting the downregulation of progenitor markers such as CSPG4, PDGFRA, and PTPRZ1, pari passu with the upregulation of early oligodendrocyte markers such as MBP, CNP, and MOG. Importantly, these same adult GPCs were found to acquire an expression signature indicative of a loss of proliferative competence coupled with upregulation of an ensemble of senescence-linked genes

Our analysis predicted that MYC, whose expression was enriched in fetal GPCs, is a central regulator of proliferative capacity of human GPCs, through its transcriptional regulation of a set of downstream genes and miRNAs that coordinately and positively regulate mitotic competence and cell cyclicity. MYC has previously been identified as an important modulator of both the epigenetic landscape and proliferation of murine GPCs, via the activation of CDK1. Moreover, MYC has recently been extensively studied as mitogenic for adult murine GPCs and an inhibitor of their senescence, functions consistent with the MYC-regulated targets of the repressive network that inventors have identified in human GPCs. Indeed, the model described herein suggests the direct repression in adult GPCs not only of MYC, but also of many of its targets as well. Interestingly, IKZF3 has been reported to directly suppress MYC in pre-B-cells, limiting their proliferative ability. For its part, MAX can complex with MYC to both inhibit its function, and to alter its transcriptional targets. Furthermore, MAX and E2F6 can both target MYC binding sites competitively, in addition to the E2F sites that E2F6 typically represses. MYC's down-regulation has also been reported to follow the upstream activation of AHR and BIN1, each of which was upregulated in the adult GPC dataset. MYC was also predicted to activate an ensemble of miRNAs in fetal GPCs, many of which were predicted to be counter-regulated by E2F6 and MAX in adult GPCs. Among these were miR-9 as well as miR-130a-3p, each of which has been previously linked to delaying senescence.

Interestingly, miR-130a-3p was also predicted to repress another highly active adult GPC transcriptional activator, STAT3, whose expression is necessary for glial development, remyelination, and has been implicated as a driver of senescence. Indeed, miRNA-130a-3p repression of STAT3 delays senescence in renal tubular epithelial cells, as driven by metformin, a drug similarly shown to enhance remyelination by aged rat GPCs. Furthermore, STAT3 expression may increase in GPCs after exposure to conditioned media taken from cultures of iPSC-derived neural progenitor cells, generated from patients with primary progressive multiple sclerosis. Beyond this, inventors predicted STAT3 activation of a cohort of miRNAs that included miR-23b-3p, the most highly upregulated miR in senescent mesenchymal stem cells.

Further assessment of the miR differential expression data revealed a number of post-transcriptional regulatory mechanisms poised to modulate fetal and adult GPC transcription. This included the upregulation in adult hGPCs of the well-studied regulators of oligodendrocyte maturation, miR-219 and miR-338, consistent with the more mature oligodendrocytic transcriptional signature of adult GPCs. In that regard, the adult GPC-enriched miRNAs miR-338-5p, miR-219a-2-3p, and miR-584-5p, have all previously been reported to be among the most highly upregulated miRs in the white matter of multiple sclerosis (MS) patients, compared to healthy controls. Accordingly, those miRNAs found to be down-regulated in MS white matter, miR-130a-3p, miR-9-3p, miR-9-5p, were also down-regulated in the adult hGPC miRNA panel. Several additional miRNAs, including miR-17-5p and miR-93-3p were also predicted by the analysis here to participate in maintaining the progenitor state of fetal GPCs, while miR-584-5p, miR-330-3p, miR-23b-3p, and miR-140-3p were predicted to promote senescence in adult GPCs.

The heterogeneity of adult hGPCs has been postulated to increase in the adult brain in a region specific manner and as such, future studies incorporating scRNA-sequencing from multiple regions, paired with spatial transcriptomics, will be needed to better understand the regional geography of normal glial aging, and its relationships with neuronal activity and vascular health. The transcriptional correlates to glial aging in the setting of disease, both neurodegenerative and dysmyelinating, will then be needed to assess the interaction of pathology with normal aging, as well as the response of aging cells to the broad variety of disease processes to which they may be exposed. In this regard, it will be critical to account for the effects of non-cell autonomous drivers of GPC aging, such as diminished local vascular perfusion and astrocytic support, on glial aging and senescence. Taken together, given the clear distinctions between young and aged hGPCs, and the extent to which their transcriptomes can be regulated via the mechanisms inventors have described, it now seems a feasible goal to safely rejuvenate aged human GPCs to a more expansion-competent and phenotypically-malleable phenotype, enabling them to more effectively compensate for the ill effects of aging and adult white matter disease.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

The foregoing examples and description of the preferred embodiments should be taken as illustrating, rather than as limiting the present disclosure as defined by the claims. As will be readily appreciated, numerous variations and combinations of the features set forth above can be utilized without departing from the present disclosure as set forth in the claims. Such variations are not regarded as a departure from the scope of the disclosure, and all such variations are intended to be included within the scope of the following claims. All references cited herein are incorporated by reference in their entireties.

1. A method of treating in a subject a condition mediated by age-related oligodendrocyte loss, said method comprising administering a therapeutically effective amount of a population of isolated glial progenitor cells to the subject in need thereof.

2. The method of claim 1, wherein the condition is a vascular leukoencephalopathy, an adult-onset autoimmune demyelination condition, a chronic post-radiation induced demyelination condition, an adult-onset lysosomal storage disease, an adult-onset leukodystrophy, or cerebral palsy.

3. A method of treating in a subject a condition mediated by age-related astrocyte loss, said method comprising administering a therapeutically effective amount of a population of isolated glial progenitor cells to the subject in need thereof.

4. The method of claim 3, wherein the condition is amyotrophic lateral sclerosis, frontotemporal dementia, schizophrenia, Huntington disease, Alexander disease, or Vanishing White Matter Disease.

5. A method of treating in a subject a condition mediated by age-related white matter loss, said method comprising administering a therapeutically effective amount of a population of isolated glial progenitor cells to the subject in need thereof.

6. The method of claim 5, wherein the condition is a vascular leukoencephalopathy, an adult-onset autoimmune demyelination condition, a chronic post-radiation induced demyelination condition, an adult-onset lysosomal storage disease, an adult-onset leukodystrophy, cerebral palsy, amyotrophic lateral sclerosis, frontotemporal dementia, schizophrenia, Huntington disease, Alexander disease, or Vanishing White Matter Disease.

7. The method of claim 5, wherein the condition is Huntington's disease or subcortical dementia.

8. The method of claim 6, wherein the vascular leukoencephalopathy is subcortical stroke, diabetic leukoencephalopathy, or hypertensive leukoencephalopathy.

9. The method of claim 6, wherein the adult-onset autoimmune demyelination condition is relapsing-remitting multiple sclerosis, chronic or progressive multiple sclerosis, neuromyelitis optica, transverse myelitis, or optic neuritis.

10. The method of claim 5, wherein the population of the isolated glial progenitor cells are younger than glial progenitor cells, oligodendrocytes, or astrocytes in the subject.

11. The method of claim 5, wherein the population of the isolated glial progenitor cells or progenies thereof replace at least some of glial progenitor cells, oligodendrocytes, or astrocytes in the subject.

12. The method of claim 5, wherein the population of the isolated glial progenitor cells or progenies thereof grow or proliferate or divide faster than glial progenitor cells, oligodendrocytes, or astrocytes in the subject.

13. The method of claim 5, wherein the population of the isolated glial progenitor cells or progenies thereof have a higher level of MYC and YAP1 pathway activity than glial progenitor cells, oligodendrocytes, or astrocytes in the subject.

14. The method of claim 5, wherein the subject is a human.

15. The method of claim 5, wherein the population of the isolated glial progenitor cells are derived from pluripotent stem cells.

16. The method of claim 15, wherein the pluripotent stem cells are embryonic stem cells.

17. The method of claim 15, wherein the pluripotent stem cells are induced pluripotent stem cells.

18. The method of claim 5, wherein said administering is carried out by intraparenchymal, intracallosal, intraventricular, intrathecal, intracerebral, intracisternal, or intravenous transplantation.

19. The method of claim 5, wherein the population of isolated glial progenitor cells are administered to the forebrain, striatum, and/or cerebellum.

20. The method of claim 5, wherein the isolated glial progenitor cells are heterologous, xenogenic, allogeneic, isogenic, or autologous to the subject.
